SEQUENCE LISTING (12 sequences)

SEQ ID NO: 1 - protein, 613 aa, Homo sapiens
EEKKVCQGTSNKLTQLGTFEDHFLSLQRMFNNCEVVLGNLEITYVQRNYDLSFLKTIQEVAGYVLIALNTVERIPLENLQIIRGNMYYENSYALAVLSNYDANKTGLKELPMRNLQEILHGAVRFSNNPALCNVESIQWRDIVSSDFLSNMSMDFQNHLGSCQKCDPSCPNGSCWGAGEENCQKLTKIICAQQCSGRCRGKSPSDCCHNQCAAGCTGPRESDCLVCRKFRDEATCKDTCPPLMLYNPTTYQMDVNPEGKYSFGATCVKKCPRNYVVTDHGSCVRACGADSYEMEEDGVRKCKKCEGPCRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENKCKATGQVCHALCSPEGCWGPEPRDCVSCRNVSRGRECVDKCKLLEGEPREFVENSECIQCHPECLPQAMNITCTGRGPDNCIQCAHYIDGPHCVKTCPAGVMGENNTLVWKYADAGHVCHLCHPNCTYGCTGPGLRGCPT

SEQ ID NO: 2 - protein, 94 aa, Homo sapiens [sequence omitted]

SEQ ID NO: 3 - protein, 214 aa, Homo sapiens
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 4 - protein, 555 aa, Homo sapiens
TQVCTGTDMKLRLPASPETHLDMLRHLYQGCQVVQGNLELTYLPTNASLSFLQDIQEVQGYVLIAHNQVRQVPLQRLRIVRGTQLFEDNYALAVLDNGDPLSPGGLRELQLRSLTEILKGGVLIQRNPQLCYQDTILWKDIFHKNNQLALTLIDTNRSRACHPCSPMCKGSRCWGESSEDCQSLTRTVCAGGCARCKGPLPTDCCHEQCAAGCTGPKHSDCLACLHFNHSGICELHCPALVTYNTDTFESMPNPEGRYTFGASCVTACPYNYLSTDVGSCTLVCPLHNQEVTAEDGTQRCEKCSKPCARVCYGLGMEHLREVRAVTSANIQEFAGCKKIFGSLAFLPESFDGDPASNTAPLQPEQLQVFETLEEITGYLYISAWPDSLPDLSVFQNLQVIRGRILHNGAYSLTLQGLGISWLGLRSLRELGSGLALIHHNTHLCFVHTVPWDQLFRNPHQALLHTANRPEDECVGEGLACHQLCARGHCWGPGPTQCVNCSQFLRGQECVEECRVLQGLPREYVNARHCLPCHPECQPQNGSVTCFGPEADQCVA

SEQ ID NO: 5 - protein, 211 aa, Homo sapiens
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNR

SEQ ID NO: 6 - protein, 220 aa, Homo sapiens
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPK

SEQ ID NO: 7 - protein, 213 aa, Homo sapiens
DIQLTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGE

SEQ ID NO: 8 - protein, 218 aa, Homo sapiens
EVQLVESGGGLVQPGGSLRLSCAASGYDFTHYGMNWVRQAPGKGLEWVGWINTYTGEPTYAADFKRRFTFSLDTSKSTAYLQMNSLRAEDTAVYYCAKYPYYYGTSHWYFDVWGQGTLVTVSSASTKGPSVFPLAPSGTAALGCLVKDYFPEPVTVSWNSGALTSGVHTFPAVLQSSGLYSLSSVVTVPSSSLGTQTYICNVNHKPSNTKVDKKVEPK

SEQ ID NO: 9 - protein, 214 aa, Homo sapiens; identical to SEQ ID NO: 3

SEQ ID NO: 10 - protein, 220 aa, Homo sapiens
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEP

SEQ ID NO: 11 - protein, 214 aa, Homo sapiens
DIQMTQSPSSLSASVGDRVTITCKASQDVSIGVAWYQQKPGKAPKLLIYSASYRYTGVPSRFSGSGSGTDFTLTISSLQPEDFATYYCQQYYIYPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 12 - protein, 222 aa, Homo sapiens
EVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

(19) United States
(12) Patent Application Publication
(10) Pub. No.: US 2008/0015194 A1
(43) Pub. Date: Jan. 17, 2008

(54) METHODS AND COMPOSITIONS OF TARGETED DRUG DEVELOPMENT

(75) Inventors: Joseph Peter Errico, Green Brook, NJ (US); Benjamin B. Mugrage, Cranbury, NJ (US); Ignatius Joseph Turchi, Yardley, PA (US)

Correspondence Address: SONNENSCHEIN NATH & ROSENTHAL LLP, P.O. BOX 061080, WACKER DRIVE STATION, SEARS TOWER, CHICAGO, IL, 60606-1080, US

(73) Assignee: Joseph Peter Errico, Green Brook, NJ (US)

(21) Appl. No.: 11/626,324

(22) Filed: Jan. 23, 2007

Related U.S. Application Data
(60) Provisional application No. 60/761,123, filed on Jan. 23, 2006.

(51) Int. Cl. A61K 31/5375; A61K 31/13; A61K 31/195; A61K 31/34; A61K 31/352; A61K 31/357; A61K 31/40; A61K 31/41; A61P 43/00; G01N 33/68; G01N 33/53; C12Q 1/70; C12Q 1/48; C12Q 1/34; A61K 31/426; A61K 31/44; A61K 31/445; A61K 31/495; A61K 31/505; A61K 31/52; A61K 31/53

(52) U.S. Cl. 514/237.8; 435/17; 435/18; 435/5; 435/7.1; 436/501; 436/86; 514/245; 514/252.12; 514/256; 514/263.1; 514/274; 514/326; 514/357; 514/369; 514/381; 514/425; 514/427; 514/452; 514/456; 514/461; 514/562; 514/563; 514/615; 514/638

(57) ABSTRACT

The present invention is directed to methods for developing one or more drugs for one or more targeted therapies and compositions derived therefrom. In accordance with one aspect of the present invention, combinatorial chemistry techniques for use with high throughput screening techniques for identifying small molecule affinity and/or activity interactions are avoided by instead utilizing the natural mechanisms of antigen response to effect a massively parallel screening of naturally occurring molecules against an antigen. Other aspects of the invention provide compositions derived therefrom as well as therapeutic methods of use for the compounds.

### CROSS-REFERENCE TO RELATED APPLICATIONS

This application claims priority from U.S. Provisional Application Ser. No. 60/761,123, filed on Jan. 23, 2006, which is incorporated herein by reference in its entirety.

### INCORPORATION-BY-REFERENCE OF MATERIAL SUBMITTED ON A COMPACT DISC

The Sequence Listing, which is a part of the present disclosure, includes a computer readable form and a written sequence listing comprising nucleotide and/or amino acid sequences of the present invention. The sequence listing information recorded in computer readable form is identical to the written sequence listing. The subject matter of the Sequence Listing is incorporated herein by reference in its entirety.

### FIELD OF THE INVENTION

The present invention generally relates to development of new chemical entities for use in the treatment of disease, and more particularly to methods of identifying lead molecules for use in quasi-rational drug design.

### BACKGROUND

Typical drug development in the modern pharmaceutical world relies on the development of models, or assays, of targeted biochemical functions. These assays are then exposed to various small molecules, some of which may be collected from the natural world, or they may be entirely synthesized in a laboratory. Without further knowledge, it can take literally thousands or millions of separate chemical exposures before a viable candidate lead molecule is identified. This process is entirely random, and is, in fact, referred to as random screening. For obvious reasons, there is no rational molecular design associated with this process, and therefore, the ten thousandth molecule tested against the assay has no greater probability of being effective than the first.

Since this form of the screening process is random, the average time to reaching success is only shortened by accelerating the rate at which the various chemicals to be tested can be gathered and exposed to the assay through, for example, high throughput screening and combinatorial chemistry has evolved.

The principle deficiency of this type of methodology, beyond the inherent randomness of it, is that the number of possible drug-like chemical structures has been estimated to be greater than ten to the eightieth power. Even using the combined power of high throughput screening and combinatorial chemistry, therefore, it is unlikely that even one chemical entity in ten to the seventieth power will ever be synthesized, much less screened. The concept of combinatorial chemistry is still valuable, inasmuch as it introduces a degree of parallel processing into the otherwise serial nature of screening. However, the limits of scalability are such that even screening a few hundred distinct chemical entities requires reversion to partially serialized testing because of the physical limits of space on a single tray.

One way that drug manufacturers have continued to develop new drugs without having to screen is to use an already approved drug as the lead for future additions to the class. That is, an FDA approved substance is used to find out if modifications can be made to it for the purposes of enhancing its potency, decreasing its side effects, or making it easier to take. For this reason, many drugs within a class are very similar. This is the case as it stands to reason that most small molecule drugs have only a specific target region (for example a protein) for effective interaction, and so long as the portion that engages with that target is conserved, other molecules may demonstrate similar activity.

For example, there are more than a half dozen different beta-blockers presently on the market. The chemical structures for six of the most widely prescribed versions of this drug class are provided in FIG. 1. FIG. 2 shows the chemical scaffold, generally referred to as the pharmacophore, that is substantially common to all of the members of this group.

This sort of grouping of drugs around a similar scaffold is not uncommon, nor is it irrational; however, the attempted modifications to the original (or “first-in-class”) drug during the development of even these follow-on drugs are often also random.

It is this fact, that a target protein or other biochemical structure usually has one surface region that can be engaged by a drug to produce a desired effect, that has led to a variety of different rational drug design techniques. Rational drug development is a process of developing lead molecules, not by randomly screening thousands of molecules in the blind hope of finding one that shows the desired activity, but rather by deducing the active site of the target and devising a chemical that interacts with that site in the appropriate manner. This strategy has experienced moderate success, however, the complexity of the chemical interaction potential makes it an extraordinarily difficult process. When successful, however, it generally results in a first-in-class drug, which often experiences a longer period of market dominance, as competitive drug makers cannot begin the copying process until the drug structure is published.

An example of a drug that has been produced by a rational drug design is Imatinib mesylate, which is a tyrosine kinase enzyme inhibitor. Tyrosine kinase enzymes are a class of molecular structures that phosphorylate the amino acid tyrosine in specific proteins. Phosphorylation is a critical modification necessary for signaling proteins, including ones that, when unregulated, can play a role in the proliferation of cancer cells (especially in certain types of leukemia). By identifying and characterizing the region of tyrosine kinase activity in the ABL-BCR (a chimeric gene encoding a tyrosine kinase, which allows the cells to proliferate without being regulated by cytokines, which in turn allows the cell to become cancerous), a small molecule was designed that would likely have the desired inhibitory activity.

While rational drug development is a very promising technique in that, when successful, it can produce first-in-class drugs, it is a very knowledge-intensive strategy. Computer modeling software presently available is only now becoming sufficient to predict the interactions of small molecules with proteins with enough accuracy to make this method viable.

It is also true that rational drug development often delays the simple screening of molecules for basic desired activity until after considerable time and expense are invested. This can lead to molecules that appear on the computer to engage a target in a desired manner, but show little if any in vitro promise. In order to avoid this, many corporate advocates of rational drug development have retreated to using the techniques as an in silico screen whereby known chemical entities (including already available drugs) are modeled and screened against the modeled target in the computer. This, of course, eliminates one of the primary advantages of the technique, which is the freedom from the bias toward already known molecules.

These disadvantages have driven some companies that had invested heavily in rational drug development back to the random screening techniques of the past. Many companies have, in fact, not even taken rational drug design seriously, and have left it to universities and national laboratories to advance the technology for them.

By the same token, the disadvantages of the combination of high throughput screening and combinatorial chemistry approach are clearly first and foremost the resource intensiveness of the technique, and second the fact that corporate realities drive much of the development away from first-in-class drug development to iterative improvements for the treatment of the same conditions.

The art would benefit from a method of drug lead identification and development that combines the advantages of high throughput screening and combinatorial chemistry, i.e., the ability to test many thousands of chemical entities to find a strongly acting candidate, with the advantages of rational drug design, i.e., the potential of developing first-in-class drugs at a reduced cost.

### SUMMARY OF THE INVENTION

Among the various aspects of the present invention is the provision of a method that can test literally trillions of chemical structures within a living host to find chemical structures that bind to the target (e.g., a protein or other large molecule); uses standard assaying techniques to determine which of the chemical structures that bind to the target will provide the desired activity; and/or uses already known facts about the binding chemical structures to guide the construction of the small molecule lead.

One aspect of the invention is directed to a method for producing a molecular structure having a desired pharmaceutical activity relative to a target biomolecule. Such method includes the steps of providing at least one immune system protein that specifically binds to a target biomolecule; determining the identity and spatial orientation of at least a portion of atoms of the immune system protein, wherein interaction of the at least a portion of atoms of the immune system protein with a binding site of the target biomolecule result in binding thereto; and constructing a pharmacophore, wherein the pharmacophore comprises a model of at least one pharmacophoric feature that approximates at least a portion of the identity and spatial orientations of the atoms of the immune system protein that specifically bind to the immune system protein such that the pharmacophore structural features are complementary to the binding site of the target biomolecule.

In various embodiments of the above aspect, the method can further include the step of identifying a candidate molecule with a pharmacophore hypothesis query of a database of annotated ligand molecules, wherein an identified candidate compound has a structure that substantially aligns with at least one pharmacophoric feature. In various embodiments of the above aspect, the method can further include the step of determining a docking affinity of the candidate molecule for the binding site of the target biomolecule; wherein docking affinity is quantified by energy gained upon interaction of the candidate molecule with the target biomolecule, energy required to attain the docked conformation relative to the lowest energy conformation, or a combination thereof.

In various embodiments, the immune system protein has an ability to alter an activity of the target biomolecule. For example, the immune system protein can have an ability to inhibit an activity of the target biomolecule.

In various embodiments, the step of providing immune system protein that specifically binds to a target biomolecule and has the ability to alter the activity of the target biomolecule includes the steps of providing an assay in which the target biomolecule displays an activity that mimics an in vivo activity; exposing a plurality of immune system proteins having a binding affinity for the target biomolecule to the target biomolecule in the assay; and selecting at least one immune system protein having the ability to alter the activity of the target biomolecule within the assay.

In various embodiments, the immune system protein that specifically binds to the target biomolecule also binds to at least one related biomolecule that differs from the target biomolecule in portions thereof, but wherein similar or identical portions of the structure and activity of the target molecule are retained by the related biomolecule. In various embodiments, the immune system protein is a major histocompatibility complex, a T-cell receptor, a β-cell receptor, or an antibody, preferably a monoclonal antibody.

In various embodiments, determining the identities and spatial orientations of at least a portion of the atoms of the monoclonal antibody includes determining the identities and spatial orientations of at least a portion of the atoms of a binding tip of the monoclonal antibody, preferably a substantial portion of the atoms of the binding tip of the at least one monoclonal antibody.

In various embodiments, the pharmacophore features include at least one feature selected from the group of hydrophobic, aromatic, a hydrogen bond acceptor, a hydrogen bond donor, a cation, and an anion features.

In various embodiments, the target biomolecule is a protein, preferably, an enzyme, a signaling protein, or a receptor protein.

In various embodiments, the target biomolecule is selected from: the causative agent of Foot and Mouth Disease, Angiotensin II; ErbB2; Flu Agglutinin; Flu Hemagglutinin; Flu Neuraminidase; Gamma Interferon; HER2; Neisseria Meningitidis; HIV1 Protease; HIV-1 Reverse Transcriptase; Rhinovirus; platelet fibrinogen receptor; Salmonella oligosaccharide; TGF-α; Thrombopoietin; Tissue Factor; Von Willenbrand Factor; VEGF; Coronavirus (SARS); the causative agent of Lyme Disease, HIV GP120; HIV GP41; West Nile Virus; Dihydrofolate reductase; and EGFR. Preferably, the taregt biomolecule is EGFR, VEGF, HER2, and ErbB2, most preferably, EGFR.

In various embodiments, determining the identities and spatial orientations of at least a portion of atoms of the at least one immune system protein includes analysis of X-ray crystallographic data derived from a crystalline form of the at least one immune system protein, preferably a crystalline form of the at least one immune system protein bound to the target biomolecule.

In various embodiments, determining the identity and spatial orientation of at least a portion of atoms of the one immune system protein includes determining the peptide sequence of the at least one immune system protein; producing a virtual model of the three dimensional structure of the immune system protein; and analyzing the virtual model of the three dimensional structure of the immune system protein so as to determine the identity and spatial orientation of at least a portion of atoms of the at least one immune system protein that interacts with a binding site of the target biomolecule resulting in binding thereto.

In one embodiment, the method for producing a molecular entity having a desired pharmaceutical activity relative to a target biomolecule, includes the steps of: (i) providing at least one monoclonal antibody; wherein the at least one monoclonal antibody specifically binds to a target biomolecule and inhibits an activity of the target biomolecule; wherein the at least one monoclonal antibody comprises a binding tip; and wherein the binding tip comprises a plurality of atoms that interact with a binding site of the target biomolecule resulting in binding thereto; (ii) determining identity and spatial orientation of a substantial portion of the binding tip atoms that interact with the binding site of the target biomolecule; wherein such determination of identity and spatial orientation comprises analysis of X-ray crystallographic data derived from a crystalline form of the at least one monoclonal antibody bound to the target biomolecule; (iii) constructing a pharmacophore; wherein the pharmacophore comprises a plurality of pharmacophoric features; wherein the plurality of pharmacophoric features approximate the identity and spatial orientation of at least about 75% of the at least one monoclonal antibody binding tip atoms that interact with the binding site of the target biomolecule; wherein the plurality of pharmacophoric features are complementary to the binding site of the target biomolecule; and wherein the plurality of pharmacophoric features comprise at least one feature selected from the group consisting of hydrophobic, aromatic, a hydrogen bond acceptor, a hydrogen bond donor, a cation, and an anion; and (iv) identifying a candidate molecule with a pharmacophore hypothesis query of a database of annotated ligand molecules; wherein an identified candidate compound has a structure that substantially aligns with at least one feature of the pharmacophore; wherein the candidate molecule inhibits the activity of the target biomolecule; and wherein the target biomolecule is an enzyme, a signaling protein, or a receptor protein.

Another aspect of the invention is directed to a pharmaceutical composition for the inhibition of EGFR. Such pharmaceutical composition includes at least one EGFR inhibitor selected from the group consisting of Formula (1), Formula (7), Formula (14), Formula (19), and Formula (25), including stereoisomers or polymorphs thereof, and a pharmaceutically acceptable carrier or diluent. Formulas are as follows:

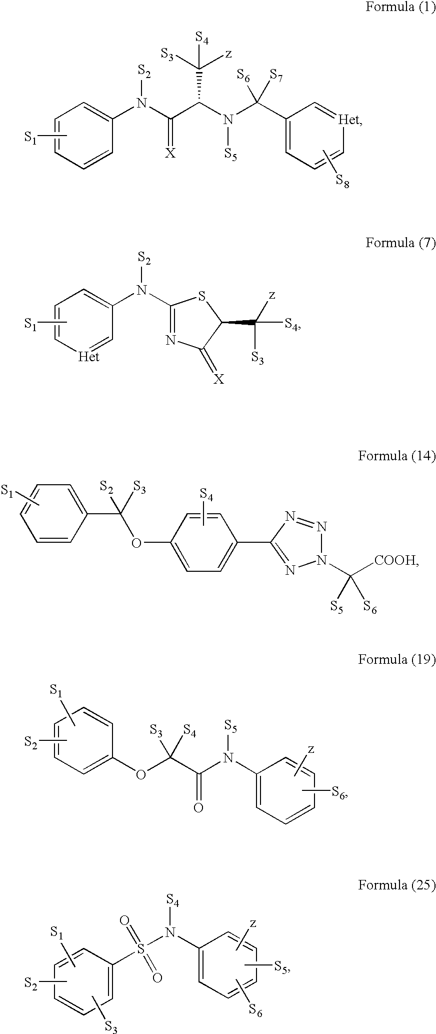

wherein S1-S8 are independently selected from the group consisting of halogen, hydroxyl, sulfhydryl, carboxylate, alkyl, cycloalkyl, aryl, and alkoxyl (—OR); X is selected from the group consisting of H₂, O, S, N—R, N—OH, and N—NR₂; Het is one or more N atoms at any ring position; Z is selected from the group consisting of —COOH, —PO₃H₂, SO₃H, tetrazole ring, sulfonamide, acyl sulfonamide, —CONH₂, and —CONR₂; and R is a C1-C6 straight chain or branched alkyl group, optionally substituted with a halogen, hydroxyl, sulfhydryl, carboxylate, aryl, heteroaryl, amino, substituted amino, or cycloamino containing one, two, or three N atoms in a 5 or 6 membered ring.

Another aspect of the invention is directed to a method for the treatment of a disease or disorder associated with EGFR including the step of administering to a mammal in need thereof a composition that includes a therapeutically effective amount of a pharmaceutical composition of the invention. Such compostions include an EGFR inhibitor selected from Formula (6); Formula (13); Formula (18); Formula (24); Formula (30), or stereoisomers or polymorphs thereof. Structures are as follows:

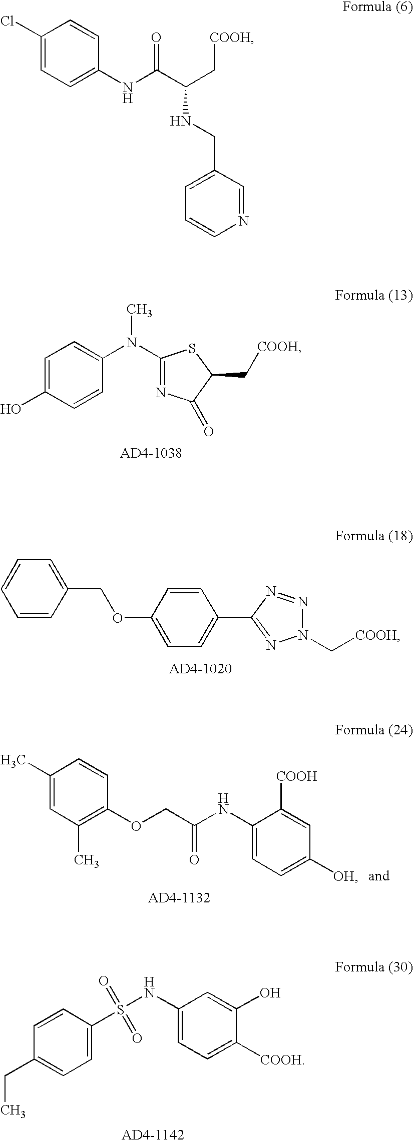

Other objects and features will be in part apparent and in part pointed out hereinafter.

### BRIEF DESCRIPTION OF THE DRAWINGS

Those of skill in the art will understand that the drawings, described below, are for illustrative purposes only. The drawings are not intended to limit the scope of the present teachings in any way.

FIG. 1A-F shows the chemical structure of atenolol, bisoprolol, metoprolol, labetalol, propranolol, and carvedilol, respectively.

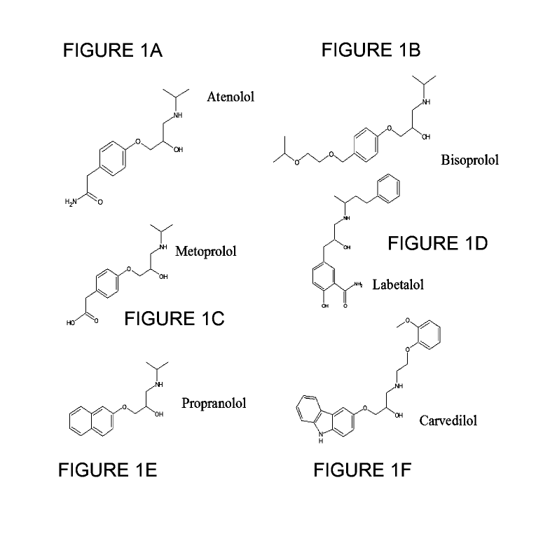

FIG. 2 shows the common chemical backbone substantially incorporated by each of atenolol, bisoprolol, metoprolol, labetalol, propranolol, and carvedilol.

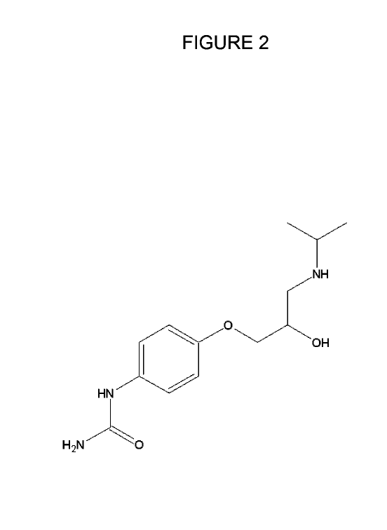

FIG. 3 is a representation of an IgG molecule.

FIG. 4 is a Jmol representation of a dimerized VEGF protein bound to two Fab antibody fragments, wherein a boxed binding region is magnified.

FIG. 5 is a ribbon model of a VEGF dimer.

FIGS. 6A and 6B are chemical structures of a lead molecule having potential activity against VEGF, wherein said lead molecule has been designed based upon the binding portion of an antibody having high affinity for VEGF as is contemplated by the methods of the present invention.

FIGS. 7A and 7B are chemical structures of a lead molecule having potential activity against hemagglutinin, wherein said lead molecule has been designed based upon the binding portion of an antibody having high affinity for hemagglutinin as is contemplated by the methods of the present invention.

FIG. 8 is a Jmol image of a Fab fragment, having high affinity for angiogenin, bound to a molecule of angiogenin, wherein a boxed region at the interface between the angiogenin molecule and the binding region of the Fab fragment is expanded.

FIGS. 9A and 9B are chemical structures of two lead molecules having potential activity against angiogenin, wherein said lead molecules have been designed based upon two closely associated binding portions of an antibody having high affinity for angiogenin as are contemplated by the methods of the present invention.

FIGS. 10A and 10B are ball and stick models of the lead molecules of FIGS. 9A and 9B, respectively.

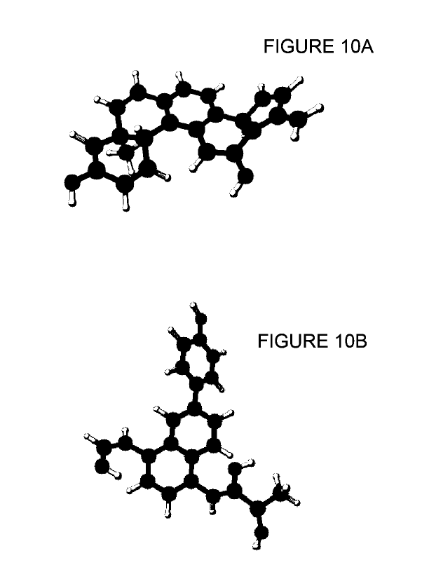

FIG. 11 depicts pharmacophore 1_gly54_asp58, derived from crystal 1YY9.pdb, superimposed on gly54_asp58 region of the antibody cetuximab.

FIG. 12 depicts pharmacophore 11_gly54_asp58, derived from crystal 1YY9.pdb, superimposed on region gly54_asp58 of the antibody cetuximab.

FIG. 13 depicts pharmacophore 21_gly54_asp58, derived from crystal 1YY9.pdb, superimposed on region gly54_asp58 of the antibody cetuximab.

FIG. 14 depicts pharmacophore 22_gly54_asp58, derived from crystal 1YY9.pdb, superimposed on region gly54_asp58 of the antibody cetuximab.

FIG. 15 depicts pharmacophore 23_gly54_asp58, derived from crystal 1YY9.pdb, superimposed on region gly54_asp58 of the antibody cetuximab.

FIG. 16 depicts pharmacophore 24_gly54_asp58, derived from crystal 1YY9.pdb, superimposed on region gly54_asp58 of the antibody cetuximab.

FIG. 17 depicts pharmacophore 1_thr100_glu10₅, derived from crystal 1YY9.pdb, superimposed on region thr100_glu105 of the antibody cetuximab.

FIG. 18 depicts pharmacophore 2_thr100_glu10₅, derived from crystal 1YY9.pdb, superimposed on region thr100_glu105 of the antibody cetuximab.

FIG. 19 depicts pharmacophore 3_thr100_glu105, derived from crystal 1YY9.pdb, superimposed on region thr100_glu105 of the antibody cetuximab.

FIG. 20 depicts pharmacophore 10_thr100_glu105, derived from crystal 1YY9.pdb, superimposed on region thr100_glu105 of the antibody cetuximab.

FIG. 21 depicts pharmacophore 21_thr100_glu105, derived from crystal 1YY9.pdb, superimposed on region thr100_glu105 of the antibody cetuximab.

FIG. 22 depicts pharmacophore 22_thr100_glu105, derived from crystal 1YY9.pdb, superimposed on region thr100_glu105 of the antibody cetuximab.

FIG. 23 depicts pharmacophore 1n, derived from crystal 1CZ8.pdb, superimposed on region tyr101_ser106 of the antibody cetuximab.

FIG. 24 depicts pharmacophore 2n, derived from crystal 1CZ8.pdb, superimposed on region tyr101_ser106 of the antibody cetuximab.

FIG. 25 depicts pharmacophore 3n, derived from crystal 1CZ8.pdb, superimposed on region tyr101_ser106 of the antibody cetuximab.

FIG. 26 depicts pharmacophore 4n, derived from crystal 1CZ8.pdb, superimposed on region tyr101_ser106 of the antibody cetuximab.

FIG. 27 depicts pharmacophore 6n, derived from crystal 1CZ8.pdb, superimposed on region tyr101_ser106 of the antibody cetuximab.

FIG. 28 depicts pharmacophore 7n, derived from crystal 1CZ8.pdb, superimposed on region tyr101_ser106 of the antibody cetuximab.

FIG. 29 depicts pharmacophore 10b, derived from crystal 1CZ8.pdb, superimposed on region tyr101_ser106 of the antibody cetuximab.

FIG. 30 depicts pharmacophore 1b, derived from crystal 1N8Z.pdb, superimposed on region arg50, tyr92-thr94, gly103 of the antibody.

FIG. 31 depicts pharmacophore 2b, derived from crystal 1N8Z.pdb, superimposed on region arg50, tyr92-thr94, gly103 of the antibody.

FIG. 32 depicts pharmacophore 2n, derived from crystal 1N8Z.pdb, superimposed on region arg50, tyr92-thr94, gly103 of the antibody.

FIG. 33 depicts pharmacophore 3n, derived from crystal 1N8Z.pdb, superimposed on region arg50, tyr92-thr94, gly103 of the antibody.

FIG. 34 depicts pharmacophore 5n, derived from crystal 1S78.pdb, superimposed on region asp31_tyr32, asn_{—}52_pro52a_asn53 of the antibody.

FIG. 35 depicts pharmacophore 6b, derived from crystal 1S78.pdb, superimposed on region asp31_tyr32, asn_{—}52_pro52a_asn53 of the antibody.

FIG. 36 depicts pharmacophore 3h, derived from crystal 2EXQ.pdb, superimposed on heavy chain tyr50_thr57 region of the antibody.

FIG. 37 depicts pharmacophore 4h, derived from crystal 2EXQ.pdb, superimposed on heavy chain tyr50_thr57 region of the antibody.

FIG. 38 depicts pharmacophore 5h, derived from crystal 2EXQ.pdb, superimposed on heavy chain tyr50_thr57 region of the antibody.

FIG. 39 depicts pharmacophore 6h, derived from crystal 2EXQ.pdb, superimposed on heavy chain tyr50_thr57 region of the antibody.

FIG. 40 depicts pharmacophore 7h, derived from crystal 2EXQ.pdb, superimposed on heavy chain tyr50_thr57 region of the antibody.

FIG. 41 depicts pharmacophore 8h, derived from crystal 2EXQ.pdb, superimposed on heavy chain tyr50_thr57 region of the antibody.

FIG. 42 depicts pharmacophore 9h, derived from crystal 2EXQ.pdb, superimposed on heavy chain tyr50_thr57 region of the antibody.

FIG. 43 depicts pharmacophore 1L and 2L (same), derived from crystal 2EXQ.pdb, superimposed on the light chain Asn32_Ile33_Gly34, Tyr49_His50_Gly51, Tyr91, Phe94, and Trp96 region of the antibody.

FIG. 44 depicts pharmacophore 3L, derived from crystal 2EXQ.pdb, superimposed on the light chain Asn32_Ile33_Gly34, Tyr49_His50_Gly51, Tyr91, Phe94, and Trp96 region of the antibody.

FIG. 45 depicts Pharmacophore 1_gly54_asp58 superimposed with residues GLY-54 to ASP-58 from the protein crystal structure of cetuximab (1YY9.pdb). Volume constraints were used to exclude the space occupied by the EGFR target protein (SEQ ID NO: 1), with a group of “dummy” spheres (dark grey) positioned to occupy the position of atoms of the target protein during a pharmacophore query. This representation is used to approximate the surface topology of the EGFR target protein.

FIG. 46 is a diagram depicting the compound AD4-1025 docked to EGFR as a 2D model with amino acid residues of EGFR annotated.

FIG. 47 is a diagram depicting the compound AD4-1038 docked to EGFR as a 3D stick model view (A) or 3D contact surface view (B).

FIG. 48 is a diagram depicting the compound AD4-1010 docked to EGFR as a 2D model with amino acid residues of EGFR annotated.

FIG. 49 is a diagram depicting the compound AD4-1009 docked to EGFR as a 2D model with amino acid residues of EGFR annotated.

FIG. 50 is a diagram depicting the compound AD4-1016 docked to EGFR as a 2D model with amino acid residues of EGFR annotated.

FIG. 51 is a diagram depicting the compound AD4-1017 docked to EGFR as a 2D model with amino acid residues of EGFR annotated.

FIG. 52 is a diagram depicting the compound AD4-1018 docked to EGFR as a 2D model with amino acid residues of EGFR annotated.

FIG. 53 is a diagram depicting the compound AD4-1020 docked to EGFR as a 2D model with amino acid residues of EGFR annotated.

FIG. 54 is a diagram depicting the compound AD4-1021 docked to EGFR as a 2D model with amino acid residues of EGFR annotated.

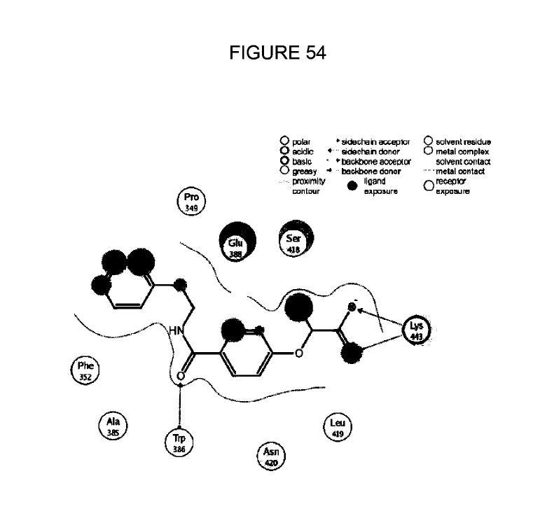

FIG. 55 is a diagram depicting the compound AD4-1022 docked to EGFR as a 2D model with amino acid residues of EGFR annotated.

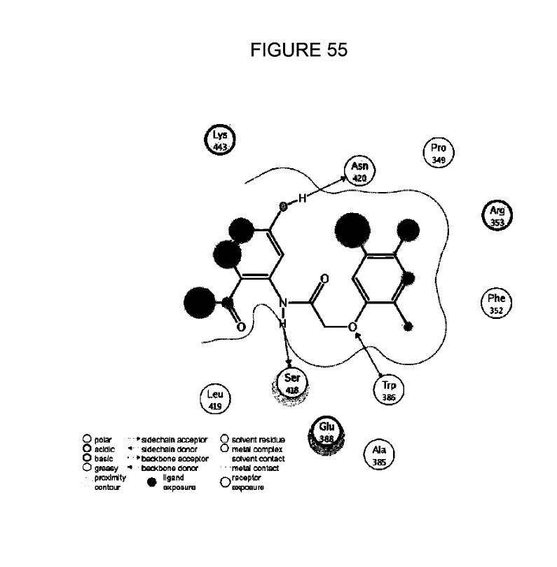

FIG. 56 is a diagram depicting the compound AD4-1027 docked to EGFR as a 2D model with amino acid residues of EGFR annotated.

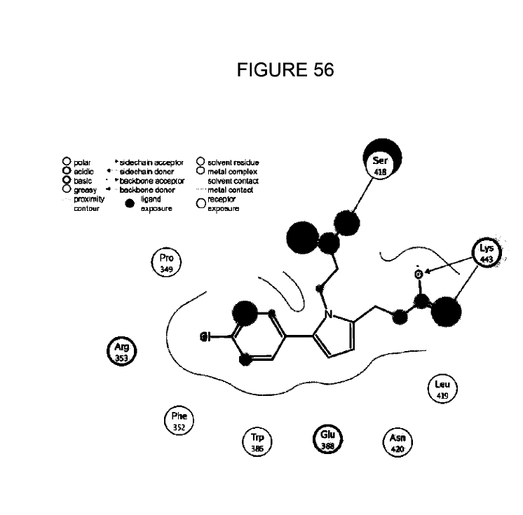

FIG. 57 is a diagram depicting the compound AD4-1030 docked to EGFR as a 2D model with amino acid residues of EGFR annotated.

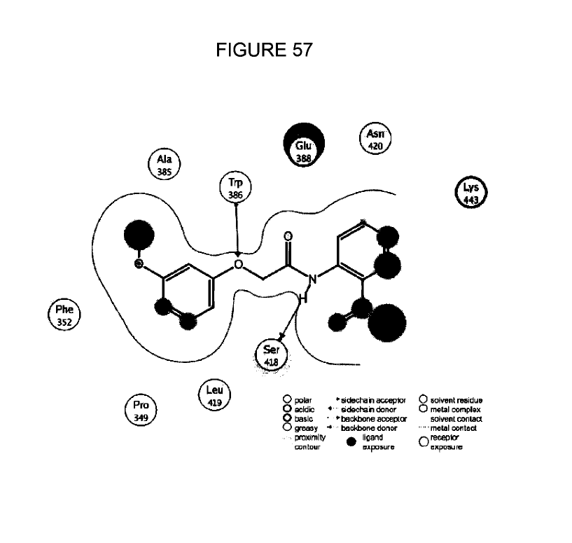

FIG. 58 is a diagram depicting the compound AD4-1132 docked to EGFR as a 3D stick model view (A) or 3D contact surface view (B).

FIG. 59 is a diagram depicting the compound AD4-1132 docked to EGFR as a 2D model with amino acid residues of EGFR annotated.

FIG. 60 is a diagram depicting the compound AD4-1142 docked to EGFR as a 3D stick model view (A) or 3D contact surface view (B).

FIG. 61 is a diagram depicting the compound AD4-1142 docked to EGFR as a 2D model with amino acid residues of EGFR annotated.

### DETAILED DESCRIPTION OF THE INVENTION

The present invention is directed to methods and apparatuses for developing one or more drugs for one or more targeted therapies. In accordance with one aspect of the present invention, combinatorial chemistry techniques for use with high throughput screening techniques for identifying small molecule affinity and activity interactions are avoided by instead utilizing the natural mechanisms of antigen response to effect a massively parallel screening of naturally occurring molecules against an antigen.

Similarly, in accordance with another aspect of the present invention, rational drug design techniques may be guided to the creation of lead molecules for pharmaceutical development based on copying the molecular substructures of biologically synthesized molecules, such as immunoglobulins, that are known to have high affinity for target structures.

In brief, a preferred embodiment of the method for developing a drug for one or more targeted therapies is as follows. Immune system proteins (e.g., an antibody, preferably a monoclonal antibody) are raised against a target biomolecule, preferably a protein, more preferably an enzyme. The binding interaction between target molecule and immune system protein is characterized, for example, via crystallography date. From the binding characterization, protein binding domains are defined. The protein binding domains can be expressed as one or more pharmacophore features and/or compiled in a pharmacophore model comprising one or more pharmacophore features. Pharmacophore features can generally be derived from corresponding moieties of the immune system protein in complex with the target biomolecule. Pharmacophore generation can be according to software designed for such a task. Candidate molecules (from, for example, one or more chemical libraries) are selected from those molecules which align to the pharmacophore models. Preferably, candidate molecules are docked and scored in silico for interaction with the target immune system protein. Again, docking and scoring can be according to software designed for such a task. After selection of molecules aligning to one or more pharmacophore models, where such molecules were optionally docked and scored in silico, the selected molecules are obtained, for example by chemical synthesis or from a commercial source. The selected molecules can be measured for binding affinity and/or effect on function for the target biomolecule. Such assessment is generally according to a biological assay. The tested molecules can be further selected according to desirable measured parameters. The selected molecules and/or the further selected molecules can optionally be further optimized.

Biomolecule Target Selection

It shall be understood that the types of biomolecule target for the lead molecules generated by the methods of the present invention can include one or more of: nucleotides, oligonucleotides (and chemical derivatives thereof), DNA (double strand or single strand), total RNA, messenger RNA, cRNA, mitochondrial RNA, artificial RNA, aptamers PNA (peptide nucleic acids) Polyclonal, Monoclonal, recombinant, engineered antibodies, antigens, haptens, antibody FAB subunits (modified if necessary) proteins, modified proteins, enzymes, enzyme cofactors or inhibitors, protein complexes, lectins, Histidine labeled proteins, chelators for Histidine-tag components (HIS-tag), tagged proteins, artificial antibodies, molecular imprints, plastibodies membrane receptors, whole cells, cell fragments and cellular substructures, synapses, agonists/antagonists, cells, cell organelles, e.g. microsomes small molecules such as benzodiazepines, prostaglandins, antibiotics, drugs, metabolites, drug metabolites natural products carbohydrates and derivatives natural and artificial ligands steroids, hormones peptides native or artificial polymers molecular probes natural and artificial receptors and chemical derivatives thereof chelating reagents, crown ether, ligands, supramolecular assemblies indicators (pH, potential, membrane potential, redox potential), and tissue samples (tissue micro arrays). The target biomolecule is preferably a protein, more preferably an enzyme.

Desirable target enzymes include those for which there exists protein-antibody crystallography data. The various methods of the invention can be used to generate pharmacophore models for a variety of protein targets (crystallized with ligand) including, but not limited to: Foot and Mouth Disease (1QGC.pdb); Angiotensin II (1CK0.pdb, 3CK0.pdb, 2CK0.pdb); ErbB2 complexed with pertuzumab antibody (1L71.pdb, 1S78.pdb, 2GJJ.pdb); Flu Agglutinin (1DN0.pdb, 1OSP.pdb); Flu Hemagglutinin (1EO8.pdb, 1QFU.pdb, 2VIR.pdb, 2VIS.pdb, 2VIT.pdb, 1KEN.pdb, 1FRG.pdb, 1HIM.pdb, 1HIN.pdb, 11FH.pdb); Flu Neuraminidase (NC10.pdb, 1AI4.pdb, 1NMB.pdb, 1NMC.pdb, 1NMA.pdb, 1 NCA.pdb, 1 NCD.pdb, 2AEQ.pdb, 1 NCB.pdb, 1 NCC.pdb, 2AEP.pdb); Gamma Interferon (HuZAF.pdb, 1T3F.pdb, 1B2W.pdb, 1B4J.pdb, 1T04.pdb); HER2 complexed with Herceptin (1N8Z.pdb, 1FVC.pdb); Neisseria Meningitidis (1MNU.pdb, 1 MPA.pdb, 2 MPA.pdb, 1UWX.pdb); HIV1 Protease (1JP5.pdb, 1CL7.pdb, 1MF2.pdb, 2HRP.pdb, 1SVZ.pdb); HIV-1 Reverse Transcriptase (2HMI.pdb, 1J5O.pdb, 1N5Y.pdb, 1N6Q.pdb, 1HYS.pdb, 1C9R.pdb, 1HYS.pdb, 1R08.pdb, 1T04.pdb, 2HRP.pdb); Rhinovirus (1FOR.pdb, 1RVF.pdb, 1BBD.pdb, 1A3R.pdb, 1A6T.pdb); platelet fibrinogen receptor (1TXV.pdb, 1TY3.pdb, 1TY5.pdb, 1TY6.pdb, 1TY7.pdb); Salmonella oligosaccharide (1MFB.pdb, 1MFC.pdb, 1MFE.pdb); TGF-Alpha (1E4W.pdb, 1E4X.pdb); Thrombopoietin complexed with TN1 (1V7M.pdb, 1V7N.pdb); Tissue Factor complexed with 5G9 (1FGN.pdb, 1AHW.pdb, 1JPS.pdb, 1UJ3.pdb); Von Willenbrand Factor complexed with NMC-4 (1OAK.pdb, 2ADF.pdb, 1FE8.pdb, 1FNS.pdb, 2ADF.pdb); VEGF complexed with B20-4 (2FJH.pdb, 2FJF.pdb, 2FJG.pdb, 1TZH.pdb, 1TZI.pdb, 1CZ8.pdb, 1BJ1.pdb); Coronavirus—SARS (2DD8.pdb, 2G75.pdb); Lyme Disease (1P4P.pdb, 1RJL.pdb); HIV GP120 (1ACY.pdb, 1F58.pdb, 1G9M.pdb, 1G9N.pdb, 1GC1.pdb, 1Q1J.pdb, 1QNZ.pdb, 1RZ7.pdb, 1RZ8.pdb, 1RZF.pdb, 1RZG.pdb, 1RZI, 1RZJ.pdb, 1RZK.pdb, 1YYL.pdb, 1YYM.pdb, 2B4C.pdb, 2F58.pdb, 2F5A.pdb); HIV GP41 (1TJG.pdb, 1TJH.pdb, 1TJI.pdb, 1U92.pdb, 1U93.pdb, 1U95.pdb, 1U8H.pdb, 1U81.pdb, 1U8J.pdb, 1U8K.pdb, 1U8P.pdb, 1U8Q.pdb, 1U91.pdb, 1U8L.pdb, 1U8M.pdb, 1U8N.pdb, 1U80.pdb, 2F5B); West Nile Virus (as defined in US Patent App. Pub. No. 2006/0115837); Malaria (Dihydrofolate reductase) (as defined in Acta Crystallographia (2004), D60(11), 2054-2057); and EGFR (1181.pdb, 118K.pdb, 1YY8.pdb, 1YY9.pdb, 2EXP.pdb, 2EXQ.pdb).

Immune System Protein Structure and Function

Immune system proteins identified as binding to the target biomolecule are used as a template to direct selection and/or construction of small organic molecule inhibitors, or pharmacophores thereof, of the target biomolecule. Generally, an immune system protein is one which binds to non-self proteins. In various embodiments, immune system proteins are raised against a target biomolecule. It is understood that multiple structures produced in the immune system express selectively high affinity for corresponding molecular structures. These include, for example, major histocompatibility complexes, various T- and β-cell receptors, and antibodies. Any one of these structures can be utilized in the steps of the present inventions; however, for the purposes of describing the preferred embodiments hereof, antibodies shall be referred to. One skilled in the art will understand that the following discussion applies to other immune system proteins as well.

Preferably, the immune system protein binds non-self proteins with little or no structural distortion caused, for example, by induced fit. It is this property of various immune system proteins that, at least in part, makes this class of molecules desirable in the methods described herein. In various embodiments, the immune system protein is at least about 95% constant in structure before and after binding, more preferably at least about 98% constant. In other words, preferable immune system proteins undergo less than about 5% or less than about 2% conformational change, as measured by the spatial position of atoms, upon binding to a non-self protein target. For example, immune system proteins of various embodiments undergo average atomic spatial movement of less than about 3 Å or less than about 2 Å after binding to a biomolecule target.

With respect to immunoglobulins, which are an aspect of a preferred method of this invention, every single healthy mammal can produce upwards of ten to the tenth different and distinct antibodies, each responding to a different antigen. Across species and even across the animal kingdom, variability in the intra-species genetic codes (specifically for the complementarity defining regions (CDR) components of antibodies) and the form of antibodies (the overall structures being monomeric, e.g., camels, versus dimeric, e.g., humans and mice) raise the number of possible antibody responses to greater than ten to the twentieth power. And every individual animal having a healthy immune system is capable of raising a plurality of antibodies against almost any antigen.

When a foreign molecule, for example an enzyme indigenous to another species, is injected into the body of an animal having a healthy immune system, a response of that system will be raised against the structure. During this response, millions of individual nascent β-cells, each expressing a distinct receptor that mirrors the identical antibody that β-cell will ultimately produce, are exposed to the molecule. Those β-cells that express receptors that bind tightly to the foreign molecule are caused to proliferate, thus providing a colony of cells that each produces the same antibody, which is specific for the target. Some of these β-cells are released into the body to combat the foreign substance, while other members remain within the lymph nodes, spleen and thalamus, prepared to respond with a flood of antibodies in the event that the foreign molecule is presented to the system in the future. This ability to lie in wait for the future presentation of the foreign molecule is referred to as “acquired immunity” inasmuch as it requires an initial presentation of the foreign substance before the ability to respond in the future can be acquired.

In the event that a specific molecular structure, for example a protein or more specifically an enzyme, is a contributor to the pathogenesis of a disease, a pharmaceutical agent that binds to that molecule with high specificity and/or inhibits the activity of that molecule is one route to finding a meaningful therapy (if not cure) for the disease. There are many examples of such enzymes, including reverse transcriptase of HIV, ABL-BCR tyrosine kinase of certain types of leukemia, and vascular endothelial growth factors (VEGFs) some of which are associated with tumor angiogenesis.

As described in the Background of the Invention, an oft-chosen method of identifying a lead small molecule that exhibits precisely this activity is to randomly screen many thousands of small molecules (synthesized or otherwise for this purpose) against the target molecule in the hopes that one of the small molecules will exhibit the desired functional properties. The one, or ones, that has the right characteristics is referred to as a lead, and goes on for further refinement until a drug is found. This method of screening and subsequent optimization is laborious and does not begin by using any leverage of knowledge of the target molecule or what structures might bind to it.

In contrast, the present invention capitalizes on binding affinity properties of immune system proteins so as to provide a high throughput affinity screening process. Initial presentation of a target molecule to the immune system results in antibody production by the immune system, where the production of the many different and distinct immunoglobulin structures acts as a massively parallel high throughput affinity screening process. Only the cell expressing receptors that bind to the target are chosen for proliferation. This is called clonal selection and is at the heart of the immune system's ability to produce target specific molecules, just as screening is at the very heart of the pharmaceutical lead discovery process.

In fact, this parallel between the immune system's production of antibodies in response to the presentation of a target molecule goes further than the similarity between target presentation/clonal selection and high throughput screening, in that once the β-cells that are capable of producing antibodies that bind to the target are driven to proliferate, mechanisms that subtly promote mutation (affinity maturation) are triggered. This process permits the future generations of β-cells to generate subtly different antibodies; some of which will bind to the target more tightly, while others will bind less tightly. The ones that bind more tightly are driven to proliferate more, and the ones that bind less tightly proliferate more slowly. This slow evolution toward higher binding affinity is mirrored in the pharmaceutical development of a drug by the cycles of lead optimization.

Antibodies within the scope of the invention include, for example, polyclonal antibodies, monoclonal antibodies, and antibody fragments. Numerous methods for the production, purification, and/or fragmentation of antibodies raised against target proteins/enzymes are well known in the art (see generally, Carter (2006) Nat Rev Immunol. 6(5), 343-357; Teillaud (2005) Expert Opin Biol Ther. 5(Supp. 1) S15-27; Subramanian, ed. (2004) Antibodies: Volume 1: Production and Purification, Springer, ISBN 0306482452; Lo, ed. (2003) Antibody Engineering Methods and Protocols, Humana Press, ISBN 1588290921; Ausubel et al., ed. (2002) Short Protocols in Molecular Biology 5th Ed., Current Protocols, ISBN 0471250929; Brent et al., ed. (2003) Current Protocols in Molecular Biology, John Wiley & Sons Inc, ISBN 047150338X; Coligan (2005) Short Protocols in Immunology, John Wiley & Sons, ISBN 0471715786; Sidhu (2005) Phage Display In Biotechnology and Drug Discovery, CRC, ISBN-10: 0824754662).

Polyclonal antibodies are heterogeneous populations of antibody molecules that are obtained from immunized animals, usually from sera. Polyclonal antibodies may be readily generated by one of ordinary skill in the art from a variety of warm-blooded animals, as well known in the art and described in the numerous references listed above. Further, polyclonal antibodies can be obtained from a variety of commercial sources.

Monoclonal antibodies are homogeneous populations of antibodies to a particular antigen. In contrast to polyclonal antibodies that may be specific for several epitopes of an antigen, monoclonal antibodies are usually specific for a single epitope. Generally, monoclonal antibodies are produced by removing β-cells from the spleen of an antigen-challenged animal (wherein the antigen includes the proteins described herein) and then fusing these β-cells with myeloma tumor cells that can grow indefinitely in culture. The fused hybrid cells, or hybridomas, multiply rapidly and indefinitely and can produce large amounts of antibodies. The hybridomas can be sufficiently diluted and grown so as to obtain a number of different colonies, each producing only one type of antibody. The antibodies from the different colonies can then be tested for their ability to bind to the antigen, followed by selection of the most effective.

In particular, monoclonal antibodies can be obtained by any technique that provides for the production of antibody molecules by continuous cell lines in culture such as those described in references listed above. Preferably, myeloma cell lines that have lost their ability to produce their own antibodies are used, so as to not dilute the target antibody. Preferably, myeloma cells that have lost a specific enzyme (e.g., hypoxanthine-guanine phosphoribosyltransferase, HGPRT) and therefore cannot grow under certain conditions (namely in the presence of HAT medium) are used. In such preferable embodiments, one can detect successful fusion between healthy β-cells and myeloma cells where the healthy partner supplies the needed enzyme and the fused cell can survive in HAT medium.

Monoclonal antibodies can also be generated by other methods such as phage display (see e.g., Sidhu (2005) Phage Display In Biotechnology and Drug Discovery, CRC, ISBN-10: 0824754662).

Such antibodies can be of any immunoglobulin class including IgG, IgM, IgE, IgA, IgD and any subclass thereof. A hybridoma producing a mAb of the invention may be cultivated in vitro or in vivo. The ability to produce high titers of monoclonal antibodies in vivo makes this a particularly useful method of production. Monoclonal antibodies generally have a longer terminal half life than many antibody fragments, translating into greater uptake, that can be desirable for various applications.

Preferably, the antibody is of the IgG immunoglobulin class. The following comments are directed to the preferred IgG class, but one skilled in the art will understand that the discussion may be applied to classes of other embodiments as well.

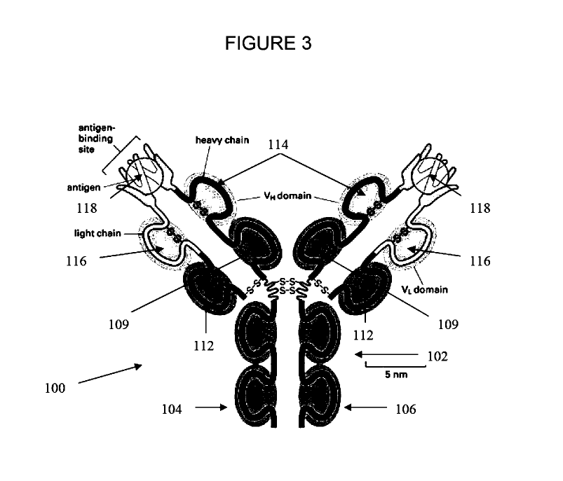

Each IgG molecule consists of two different classes of polypeptide chains, the heavy and light chains. These heavy and light chains are further subclassified as constant and variable segments. The overall construction of an IgG molecule 100 is “Y” shaped, as shown in FIG. 3, with the base 102 of the “Y” being formed by two pair of constant heavy chain segments 104, 106 (two segments, CH₂—CH₃, side by side). Each of the upper segments of this base structure is linked to one of the two branches of the “Y” 108, 110, and specifically each is connected to another constant heavy segment CH1 109. Each of these two heavy chain segments CH1 is paired with a constant light chain segment CL1 112. The distal tips of the constant heavy and light chain segments are connected to variable heavy and light chain segments VH1 114 and VL1 116 (one pair of variable segments per branch). These paired variable segments form the distal tips of the “Y” structure, and include the binding tips that are formed with such high antigen specificity. Topography of antibody binding sites is reviewed by, for example, Lee et al. (2006) J Org Chem 71, 5082-5092.

While variability in the structure of the constant segments exists across species and even some variation has been reported within species, the constant heavy segments CH1, CH2, and CH3, in mammals generally consist of a very highly conserved 110-120 amino acid sequence. Similarly, the constant light chain segments generally consist of a very highly conserved 100-110 amino acid sequence.

The light and heavy variable chain segments, VH1 and VL1, comprise very similar peptide sequences to the constant segments, but for three small peptide stretches that are approximately 5 to 15 amino acids in length. These short stretches are highly variable, and are generally referred to as the hypervariable regions or complementarity defining regions (CDRs) 118. This hypervariability is the result of genetic splicing and shuffling that occurs during the maturation of an immunoglobulin-producing cell. Each mature immunoglobulin-producing cell will produce only one type of antibody (if it is an antibody producing cell), but different cells will produce different immunoglobulins. This genetic process, therefore, gives rise to the wide variety of antibodies produced within a single animal.

The three short hypervariable peptide sequences of each of the variable segments form a complex of six amino acid groupings that bundle together at each of the distal tips of the antibody (the two distal tips being identical to one another). The antibody molecule itself, therefore, can be thought of as comprising a large structure that is dedicated to simply holding and presenting a small group of amino acids, the CDRs, in a stable arrangement so that they may bind with a very high affinity to a very specific target structure.

Because the remaining sections of the variable chain segments are highly conserved, relative to the hypervariable peptide stretches, the specific amino acids that form the CDRs can be identified by sequencing methods. Hypervariable regions of the variable light chain segment are found at, for example, peptides stretches 24-34, 50-56, and 89-97 (according to the numbering system employed by Kabat and Wu). Similarly, the hypervariable regions of the variable heavy chain segment are found at, for example, 31-35, 50-65, and 95-102. It should be understood that specific CDRs may include a larger number of peptides than would otherwise be permitted based solely on the numbers available, i.e., CDR H3 is often larger than just 8 peptides, and in these situations alphanumerics are employed, for example 100A, 100B, etc., to uniquely describe the sequence components.

Selection of Immune System Proteins

Immune system proteins are generally selected for their ability to bind the biomolecule target. Preferably, the immune system protein binds the biomolecule target with a relatively high affinity. For example, preferable immune system proteins can bind the biomolecule target with at least a K_{D} of about 1 mM, more usually at least about 300 μM, typically at least about 10 μM, more typically at least about 30 μM, preferably at least about 10 μM, and more preferably at least about 3 μM or better. Preferably, the high affinity immune system protein is a high affinity monoclonal antibody. One skilled in the art will understand that, while portions of the following discussion reference antibodies and, more specifically, monoclonal antibodies, the discussion applies also to other types of immune system protein discussed above.

Generally, binding at, in, or near the active site is a preferred embodiment given that such binding is more likely to inhibit the activity of the target biomolecule. But other embodiments are contemplated where binding of immune system proteins to regions of the target biomolecule may also result in inhibition of activity through, for example, allosteric binding (e.g., stabilization of an inactive conformation). Algorithms to identify immune system protein binding class based on the definition and site of the binding site are known to the art (see Lee et al. (2006) J Org Chem 71, 5082-5092). In accord with the vernacular of Lee et al., in various embodiments, immune system proteins can have a binding topography of cave, crater, canyon, valley, or plain. Preferably, the immune system proteins have a binding topography of canyon, valley, or plain, more preferably, canyon or plain.

Once high affinity antibody structures have been identified and monoclonal antibody producing cell lines for them have been created, a subsequent step in the method of embodiments of the present invention is to select the high affinity binding antibodies that bind at, in, or near the active site from among the plurality of antibodies (e.g., monoclonal antibodies). Monoclonal antibodies can be selected on the basis of, for example, their specificity, high binding affinity, isotype, and/or stability. Monoclonal antibodies can be screened or tested for specificity using any of a variety of standard techniques, including Western Blotting (Koren, E. et al., Biochim. Biophys. Acta 876:91-100 (1986)) and enzyme-linked immunosorbent assay (ELISA) (Koren et al., Biochim. Biophys. Acta 876:91-100 (1986)).

Methods of selecting the active site high affinity binding antibodies can be utilized when other members of a family of molecules exist, and share the same substructure of the active region thereof. One aspect of the present invention includes a method for identifying if a high affinity antibody is also an active site high affinity antibody by determining if it also binds to other members of a family of similar proteins that conserve their active region. If an high affinity antibody raised against a target molecule (e.g., VEGF-A) does not bind well to other members of the family, it is more likely that it is not binding to the active region. Alternatively, if an high affinity antibody cultivated by inoculation against a target molecule (e.g., VEGF-A) is screened against several other members of the family (e.g., VEGF-B, VEGF-C, etc.) and shows a high affinity for them as well, it is highly likely that the high affinity antibody is also an active site high affinity antibody.

In the case of molecules without similar family molecules, alternative means of determining the nature of the binding site may be employed. One example of how this determination may be made is by producing a functional assay of the target and exposing the antibody to the assay to determine if the antibody inhibits the functioning of the assay.

Another exemplary method of selecting active site high affinity antibodies from a group of high affinity antibodies, which is entirely in silico, is to sequence each antibody, model the structure of the binding surface, and to match it to a model of the active surface of the target to see if the two are compatible. This method may require knowledge of the specific target, and access to one of the several programs that are available for estimating the surface composition of antibodies. It is contemplated that this alternate method of filtering the non-active site high affinity antibodies from those antibodies that bind to the active surface target will be increasingly efficient as more target structures are fully characterized, and the accuracy of antibody modeling from sequence information alone is enhanced according to the various methods disclosed herein or otherwise. The fact that the CDRs of the antibody are known to exist at specifically enumerated stretches along the light and heavy peptide chains within the antibody provides additional reliability to this process. This in silico technique can relate to other steps (e.g., determining specific spatial position of the atoms of the binding portion of the antibody) of the inventive method as well.

Determining Structure Spatial Position

After immune system proteins (e.g., active site high affinity monoclonal antibody) are selected, 3D protein binding domains are defined. Definition of the protein binding domain(s) generally involves the determination of the specific spatial position of the atoms of the binding portion of the immune system protein that interact with the target biomolecule.

Determination of the spatial position of the binding portion can be achieved by means of various in silico techniques. For example, software packages can be used that model the structure of the binding surface and match it to a model of the active surface of the target to assess levels of compatibility. Such software includes CAMAL. Also, algorithms to identify immune system protein binding class based on the definition and site of the binding site (see Lee et al. (2006) J Org Chem 71, 5082-5092).

Alternatively, the three-dimensional positioning of atoms within a target molecule (especially a large molecule like an antibody) can be determined by crystallizing the molecule into a long array of similar structures and then exposing the crystal to X-ray diffraction. The technique of X-ray diffraction generally begins with the crystallization of the molecule because one photon diffracted by one electron cannot be reliably detected. However, because of the regular crystalline structure, the photons are diffracted by corresponding electrons in many symmetrically arranged molecules. Because waves of the same frequency whose peaks match reinforce each other, the signal becomes detectable. X-ray crystallography can provide resolution down to 2 angstroms or smaller. Techniques for employing X-ray crystallography for structural determination are known in the art (see e.g., Messerschmidt (2007) X-Ray Crystallography of Biomacromolecules: A Practical Guide, John Wiley & Sons, ISBN-10: 3527313966; Woolfson (2003) An Introduction to X-ray Crystallography, 2d Ed., Cambridge University Press, ISBN-10: 0521423597).

X-ray crystallography can be used to determine the structure of atoms within a structure that is known to bind with high affinity to the active site of a target biomolecule, and to then use this structural information to build a synthetic molecule that retains the same affinity and/or activity as the antibody.

Structural determination via X-ray crystallography requires crystals of the molecule of interest. Several techniques for creating such crystals of immune system proteins are known to the art, and include those set forth in U.S. Pat. No. 6,931,325 to Wall and U.S. Pat. No. 6,916,455 to Segelke, the specifications, teachings, and references of which are incorporated herein fully by reference. To overcome difficulties in crystallization of antibodies and potential distortion of the binding tips, the antibody can be crystallized with the target biomolecule to ensure the proper binding structure is captured (see e.g., entry 1CZ8 in the RCSB Protein Data Bank, which is a vascular endothelial growth factor in complex with an affinity matured antibody).

Once prepared, the crystals can be harvested and, optionally, cryocooled with gaseous or liquid nitrogen. Cryocooling crystals can reduce radiation damage incurred during data collection and/or decreases thermal motion within the crystal. Crystals are placed on a diffractometer coupled with a machine that emits a beam of X-rays. The X-rays diffract off the electrons in the crystal, and the pattern of diffraction is recorded on film or solid state detectors and scanned into a computer. These diffraction images are combined and used to construct a map of the electron density of the molecule that was crystallized. Atoms are then fitted to the electron density map and various parameters, such as position, are refined to best fit the observed diffraction data. Parameters derived from X-ray crystallography observed diffraction data include, but are not limited to, hydrogen bonders, apolar hydrophobic contacts, salt bridge interactions, polar surface area of the domain, apolar surface area of the domain, shape complementarily score for the antibody-target complex, and explicitly placed water molecules. Also useful is characterization of bonds between atoms. The distance between two atoms that are singly bonded ranges from about 1.45 to about 1.55 Å. Atoms that are double bonded together are typically about 1.2 to about 1.25 Å apart. Bonds that are resonant between single and double bonds typically have an about 1.30 to about 1.35 Å separation.

By way of example, a VEGF (SEQ ID NO: 2) molecule bound to an affinity-matured antibody (the Fab fragment thereof) has been previously crystallized and published by Chen, et al. in the RCSB database as 1CZ8. More particularly, their crystallization data includes regions V and W, which are the members of the VEGF dimer, and regions L, H, X, and Y which represent the antibody light and heavy chains of the Fab molecule. (More particularly, the L and H regions comprise one of the branches of the Fab molecule, including both the variable and constant regions of each chain. Similarly, X and Y are the light and heavy chains of the other branch of the Fab molecule.) By geometrically analyzing the spatial arrangement of the more than eight thousand non-hydrogen atoms of the crystalline structure, those atoms of one structure that are within a specified distance of atoms of another structure can be identified. This filter determines, by a straightforward geometric comparison across all possible combinations, the peptides that are in association across the two molecules. Using a maximum separation (e.g., 4 Å), those atoms of the heavy variable chain (H) that are within such a short range of atoms of the W component of the VEGF dimer can be determined, and are most likely to be the ones in the CDRs of the Fab fragment (see e.g., Example 1).

Construction of Pharmacophores

Immune system protein structural information, including definition of atom position, can be used to construct a pharmacophore model used to identify small molecules which have similar atoms in similar positions. Small molecules that have similar features to the immune system protein have the potential to demonstrate similar molecular interactions with the target protein and thus similar biological activity with similar therapeutic utility.

Once the identification of the spatial orientation of atoms, preferably substantially all atoms and more preferably all atoms, in the binding region(s) of an immune system protein (e.g., binding tips of an active site high affinity monoclonal antibody) has been accomplished, a subsequent step of various embodiments of the present invention is generation of a pharmacophore having a structure that approximates, preferably substantially approximates, at least a portion of the atoms of the immune system protein responsible, at least in part, for binding to the biomolecule target. For example, the pharmacophore can approximate at least about 25%, 30%, 35%, 40%, 45%, 50%, 55%, 60%, 65%, 70%, 75%, 80%, 85%, 90%, 95%, 99%, or 100% of the atoms of the immune system protein responsible, at least in part, for binding to the biomolecule target. This synthesis of a de novo chemical structure can be accomplished using rational drug design software and techniques.

One key feature of several embodiments, however, is that the lead molecule is not constructed solely by matching the new chemical structure to the target surface, but rather by employing as a guide a known structure (i.e., an immune system protein) that binds to the biomolecular target in a manner that produces the desired effect. Immune system proteins are particularly suited as guides because their CDR regions are constructed of relatively simple organic structures that can be recreated in small organic molecules relatively easily.

In various embodiments, in silico approaches can be used for de novo structure design with a fragment based approach employing contact statistics, 3D surface models, and docked ligands as templates. From the spatial position information, and/or from other parameters described above, one can derive 3D ligand-receptor models (e.g., interaction pattern, pharmacophore schemes), surface maps (e.g., topography/shape, electrostatic profile, hydrophobicity, protein flexibility), and docking models (e.g., scoring system for ligand binding, minimum energy calculation).

A pharmacophore model or scheme is generally a set of structural features in a ligand that are related, preferably directly related, to the ligand's recognition at a receptor site and its biological activity. Pharmacophore features can be derived from corresponding donor, acceptor, aromatic, hydrophobic, and/or acidic or basic moieties of the corresponding immune system protein in complex with its receptor taken from crystal structures. It shall be understood that additional information about the nature of the atoms in the immune system protein (e.g., atoms in the binding tip of an active site high affinity monoclonal antibody) being used in a pharmacophore scheme, and not simply the spatial location of the atoms, can assist in the modeling process of this new chemical lead. These characteristics include, but are not limited to, the pKa values of the atoms, the rotational rigidity of the bonds holding the atoms in place, the nature of the bonds themselves (single, double, resonant, or otherwise), the projected directionality of hydrogen bond donors and acceptors, etc.

Typical feature components useful in generating a pharmacophore scheme include, but are not limited to, atomic position; atomic radii; hydrogen bond donor features; hydrogen bond acceptor features; aromatic features; donor features; acceptor features; anion features; cation features; acceptor and anion features; donor and cation features; donor and acceptor features; acid and anion features; hydrophobic features, hydrogen bond directionality, and metal ligands (see e.g., Example 4). Such features can be located, for example, at a single atom, centroids of atoms, or at a projected directional position in space.

It is contemplated that numerous pharmacophore queries can be designed for any given immune system protein—target biomolecule complex. It is further contemplated that these pharmacophore queries will be useful to identify small molecule ligands which interact with the target biomolecule at a site recognized by the immune system protein.

Exemplary resources for accomplishing such modeling and queries include, but are not limited to MOE (CGG) (providing pharmacophore query and visualization), Glide (Schrodinger) (providing docking and scoring), Accord for Excel (Accelrys) (providing organization of molecular information including chemical structures and formulas), and the ZINC database (UCSF) (providing a library of commercial compounds). One design tool for the generation of pharmacophores from immune system protein—target biomolecule structural binding characterization is MOE, or Molecular Operating Environment (Chemical Computing Group). Model generation uses geometrical and electronic constraints to determine the 3D positions of features corresponding to the immune system protein. The model of these embodiments consists of spherical features in 3D space. The diameter of the spheres can be adjusted (e.g., about 0.5 to about 3.0 Å). Such models allow matches and/or partial matches of the features.

Pharmacophoric structural features can be represented by labeled points in space. Each ligand can be assigned an annotation, which is a set of structural features that may contribute to the ligand's pharmacophore (see e.g., Example 4). In various embodiments, a database of annotated ligands can be searched with a query that represents a pharmacophore hypothesis (see e.g., Example 5). The result of such a search is a set of matches that align the pharmacophoric features of the query to the pharmacophoric features present in the ligands of the searched database (see e.g., Example 5, Table 23-28). The number of hits within the database depends, at least in part, upon the size of the database and the restrictiveness of the pharmacophore query (e.g., partial matches, number of features, etc.). As an example, the pharmacophore queries of Example 4 generated about 1,000 to about 3,000 hits against the ZINC database. Properties and parameters of the molecules present within the search database are used to focus the outcome of the query. For example, compounds with a defined range of molecular weight (MW) or lipohilicity (logP) can be present in the searched section of the library database of compounds.

Candidate Molecules

The subject methods find use in the screening of a variety of different candidate molecules (e.g., potentially therapeutic candidate molecules). As described above, candidate molecules can be searched using a pharmacophore query. Candidate molecules encompass numerous chemical classes, though typically they are organic molecules, preferably small organic compounds having a molecular weight of more than 50 and less than about 2,500 Daltons. Candidate molecules comprise functional groups necessary for structural interaction with proteins, particularly hydrogen bonding, and typically include at least an amine, carbonyl, hydroxyl or carboxyl group, preferably at least two of the functional chemical groups. The candidate molecules often comprise cyclical carbon or heterocyclic structures and/or aromatic or polyaromatic structures substituted with one or more of the above functional groups.

In preferred embodiments, the candidate molecules are compounds in a library database of compounds. One of skill in the art will be generally familiar with, for example, numerous databases for commercially available compounds for screening (see e.g., ZINC database, UCSF, with 2.7 million compounds over 12 distinct subsets of molecules; Irwin and Shoichet (2005) J Chem Inf Model 45, 177-182). One of skill in the art will also be familiar with a variety of search engines to identify commercial sources or desirable compounds and classes of compounds for further testing (see e.g., ZINC database; eMolecules.com; and electronic libraries of commercial compounds provided by vendors, for example: ChemBridge, Princeton BioMolecular, Ambinter SARL, Enamine, ASDI, Life Chemicals etc).

Candidate molecules for screening according to the methods described herein include both lead-like compounds and drug-like compounds. A lead-like compound is generally understood to have a relatively smaller scaffold-like structure (e.g., molecular weight of about 150 to about 350 kD) with relatively fewer features (e.g., less than about 3 hydrogen donors and/or less than about 6 hydrogen acceptors; hydrophobicity character xlogP of about −2 to about 4) (see e.g., Angewante (1999) Chemie Int. ed. Engl. 24, 3943-3948). In contrast, a drug-like compound is generally understood to have a relatively larger scaffold (e.g., molecular weight of about 150 to about 500 kD) with relatively more numerous features (e.g., less than about 10 hydrogen acceptors and/or less than about 8 rotatable bonds; hydrophobicity character xlogP of less than about 5) (see e.g., Lipinski (2000) J. Pharm. Tox. Methods 44, 235-249). Preferably, initial screening is performed with lead-like compounds.

When designing a lead from spatial orientation data, it can be useful to understand that certain molecular structures are characterized as being “drug-like”. Such characterization can be based on a set of empirically recognized qualities derived by comparing similarities across the breadth of known drugs within the pharmacopoeia. While it is not required for drugs to meet all, or even any, of these characterizations, it is far more likely for a drug candidate to meet with clinical successful if it is drug-like.

Several of these “drug-like” characteristics have been summarized into the four rules of Lipinski (generally known as the “rules of fives” because of the prevalence of the number 5 among them). While these rules generally relate to oral absorption and are used to predict bioavailability of compound during lead optimization, they can serve as effective guidelines for constructing a lead molecule during rational drug design efforts such as may be accomplished by using the methods of the present invention.

The four “rules of five” state that a candidate drug-like compound should have at least three of the following characteristics: (i) a weight less than 500 Daltons; (ii) a log of P less than 5; (iii) no more than 5 hydrogen bond donors (expressed as the sum of OH and NH groups); and (iv) no more than 10 hydrogen bond acceptors (the sum of N and O atoms).

Also, drug-like molecules typically have a span (breadth) of between about 8 Å to about 15 Å. For an example of a subgroup of the atoms involved in binding that are close enough together to be structured into a lead molecule, see Table 3 of Example 1.

As explained above, the number of molecules identified as hits to the pharmacophore depend, at least in part, on the size of the database and the restrictiveness of the pharmacophore query. The number of molecules identified as hits from a pharmacophore query can be reduced by further modeling of fit to the binding site of the target biomolecule. Such modeling can be according to docking and scoring methods, as described below.

Docking and Scoring

Candidate molecules identified as having similar atoms in similar positions and/or similar features in similar positions as compared to a pharmacophore model (e.g., through a pharmacophore query as described above) can be further selected according to docking affinity for the target biomolecule (see e.g., Example 5). In addition to pharmacophore model generation for database queries, a second sequential and complementary method for compound identification and design can be employed. Pharmacophore queries can filter out compounds quickly and docking and scoring can evaluate ligand-target biomolecule binding more accurately. In the case of protein or enzyme target biomolecules, amino acid residues of the target protein or enzyme involved with antibody contact can be used to define the docking site.

In various embodiments, selected compounds from the pharmacophore queries are docked to the target protein/enzyme binding site using software designed for such analysis (e.g., Glide (Schrodinger, N.Y.). Docking affinity can be calculated as numerical values (e.g., “Glide score”) based upon, for example, energy gained upon interaction of the molecule with the protein (e.g., “g_score”) and/or energy required to attain the docked conformation relative to the lowest energy conformation (e.g., “e_model”) (see e.g., Example 5). For these particular examples, the more negative the score, the better the docking. Preferably, the g_score is less than about −5. Preferably, the e_model score is less than about −30. It is contemplated that the desirable numerical quantification of docking can vary between different target biomolecules. In various embodiments, a threshold docking score (e.g., g_score and/or e_model score) can be chosen so as to manage the number of molecules for acquisition and further testing. For example, in various docking studies described herein, for VEGF (Pdb:1 cz8) a g-score of negative 5.0 (or greater magnitude in a negative direction) was considered a desirable docking score and the cut off was adjusted accordingly; yet for ErbB2 (pdb:1s78), a g_score of negative 7.5 (or greater magnitude) was considered a desirable docking score. In these studies, the magnitude of the g_score used to adjust the number of hits to a workable number that could be acquired and tested. As an example, if the total number of compounds identified from a pharmacophore query was about 1,000 to about 3,000, the docking scores can be used to rank such compounds so as to select about 100 to about 200 for further testing. It is contemplated the number of compounds to be selected for further testing could be lower or higher than these estimates. Preferably, magnitude of the g_score is used as a selection criteria, but it is contemplated that e_model score could be similarly used, especially where e_model score is of low magnitude. It is further contemplated that the selection criteria can be based upon both g_score and e_model score, preferably weighted toward g_score.

Docking and scoring can result in a group of compounds with multiple conformers. Using suitable modeling software (e.g., MOE), 3D structures can be converted to 2D and duplicates thereby removed. The resulting list of preferred chemical structures can used to search for commercial vendors using, for example, search engines designed for such a task (e.g., eMolecules.com).

Effect on Target Biomolecule

Candidate molecules selected according to pharmacophore query and/or further selected according to docking analysis can be tested for effect on the target biomolecule. Assessment of effect of a molecule on biomolecule function (e.g., inhibition of enzymatic activity) can be assessed by various methods known in the art (see e.g., Example 6). For example, inhibitory effect of a candidate molecule on the catalytic activity of a target enzyme can be assessed by known activity assays specific for the target enzyme (see e.g., Reymond, ed. (2006) Enzyme Assays: High-throughput Screening, Genetic Selection and Fingerprinting, John Wiley & Sons, 386 p., ISBN-10: 3527310959; Eisenthall and Danson, Ed. (2002) Enzyme Assays, 2d edition, Oxford University Press, 384 p., ISBN-10: 0199638209).

Further Refinement

Several methods for further refining the selected candidate molecules. Data from biological assays can be correlated with the docking model so as to further refine lead-like molecules and/or drug-like molecules. Various software packages (e.g., MOE) can be employed to visualize active compounds in the binding site of the target biomolecule to identify sites on the template suitable for modification by de novo design. Analogs of active compounds can be identified using similarity and sub-structure searches (see e.g., SciFinder; eModel). Available analogs can be analyzed according to docking and scoring procedures described above. Analogs with desirable docking scores can be acquired and further tested for biological effect on the target biomolecule according to methods described above. One skilled in the art will understand these, and other, methods of refining and further developing candidate molecules identified by the methods presented herein.

Molecules

Another aspect of the present invention includes compounds, identified by the methods described herein, and useful for treatment of diseases, disorders, or conditions related to the target biomolecule according to which they were identified from. For example, it is well known that inhibition of growth factor proteins has a benefit in treatment of certain conditions in oncology. As another example,

AD4-1025

AD4-1025 is identified as an inhibitor of epidermal growth factor binding to its receptor (see e.g., Example 7). Such compounds have utility as treatments in oncology. Analogs and derivatives of AD4-1038 are expected to have the same inhibitory effect and utility. A pharmacophore model, Pharm1_gly54_asp58, was designed using information from the 1YY9 protein crystal structure to design a pharmacophore model (see e.g., Example 4). The Pharm1_gly54_asp58 model was utilized to identify small molecules which bind to EGFR (SEQ ID NO: 1). The site on the EGFR protein is recognized by amino acid residues GLY-54 to ASP-58 of the antibody Cetuximab (SEQ ID NO: 5 and SEQ ID NO:6) (Erbitux). Pharm1_gly54_asp58 is modeled after residues GLY-54 to ASP-58 and designed as a tool to identify small molecules which have features and components of the antibody cetuximab. Specifically this region is defined as the H2 CDR of the antibody heavy chain of cetuximab. Features and components of these amino acid residues of cetuximab were used to create a pharmacophore model. From this pharmacophore model is derived the following compound:

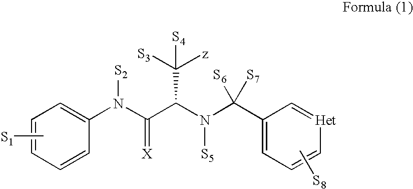

wherein S1-S8 represent independent substituents of the following type: Halogen (F, Cl, Br, I); Hydroxyl (—OH); Sulfhydryl (—SH); Carboxylate (—COOH); Alkyl (C1-C4 carbons, straight chain, branched or optionally containing unsaturation); Cycloalkyl (C1-C6 optionally containing unsaturation); Aryl including phenyl or heteroaryl containing from 1 to 4 N, O, and S atoms; or Alkoxyl (—OR where R is defined as C1-C6 straight chain or branched alkyl group, optionally substituted with halogen, hydroxyl, sulfhydryl, carboxylate, aryl, heteroaryl, amino —NH₂, substituted amino —NR₂, or cycloamino groups containing one, two, or three N atoms in a 5 or 6 membered ring); X is defined as H₂, O, S, N—R, N—OH, or N—NR₂; Het is defined as one or more N atoms at any ring position; and Z is defined as —COOH, —PO₃H₂; SO₃H, tetrazole ring, sulfonamide, acyl sulfonamide, —CONH₂ or —CONR₂.

Additional analogs include those where one or more of the nitrogen atoms are replaced with unsubstituted carbon atoms or carbon atoms containing one or two independent substituents where S9-S11 are defined as above for S1-S8:

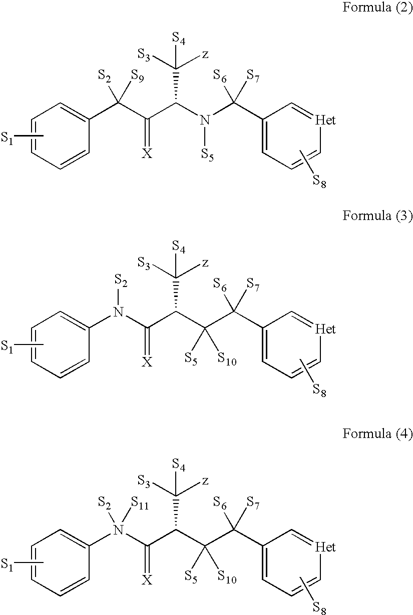

Additionally, the enantiomeric isomers are also expected to have the same utility:

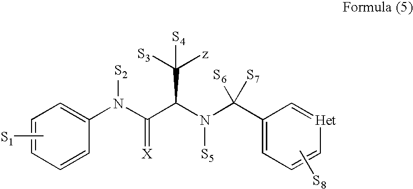

wherein S1-S8, X, Het and Z are define as above.

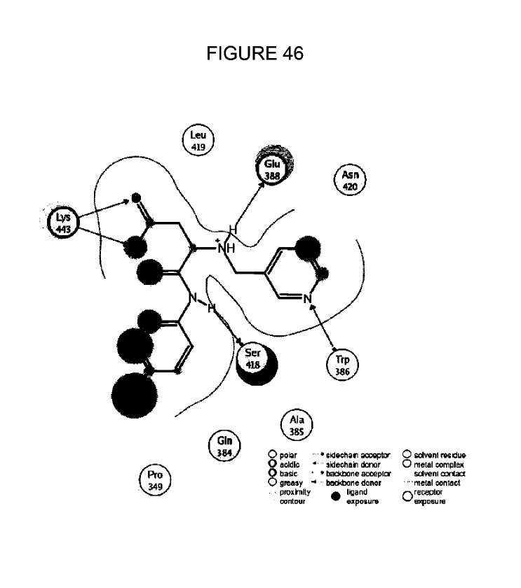

In one embodiment, the inhibitor of the binding of epidermal growth factor (EGF) to epidermal growth factor receptor (EGFR) is AD4-1025 ((N¹-(4-chlorophenyl)-N²-(3-pyridinylmethyl)-alpha-asparagine; Formula: C₁₆H₁₆ClN₃O₃; Molecular weight: 333.78)) (see e.g., Example 7). An exemplary depiction of the binding of AD4-1025 to EGFR is shown in FIG. 46. The structure of AD4-1025 is as follows:

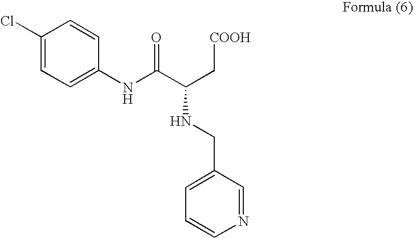

At a concentration of 25 μM of AD4-1025, binding of EGF to EGFR is inhibited by 75.7% (see e.g., Example 6).

AD4-1038

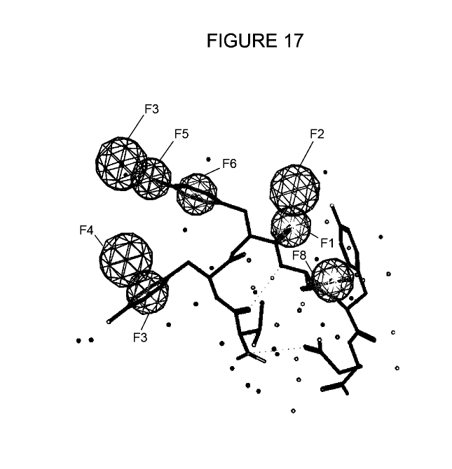

AD4-1038 is identified as an inhibitor of epidermal growth factor binding to its receptor (see e.g., Example 8). Such compounds have utility as treatments in oncology. Analogs and derivatives of AD4-1038 are expected to have the same inhibitory effect and utility. A pharmacophore model, Pharm1_thr100_glu105, was designed using information from the 1YY9 protein crystal structure to design a pharmacophore model (see e.g., Example 4; Table 17; FIG. 17). The Pharm1_thr100_glu105 model was utilized to identify small molecules which bind to EGFR. The site on the EGFR (SEQ ID NO: 1) protein is recognized by amino acid residues THR-100 to GLU-58 of the antibody Cetuximab (SEQ ID NO: 5 and SEQ ID NO:6) (Erbitux). Pharm1_thr100_glu105 is modeled after residues THR-100 to GLU-58 and designed as a tool to identify small molecules which have features and components of the antibody cetuximab. Features and components of these amino acid residues of cetuximab were used to create a pharmacophore model. From this pharmacophore model is derived the following compound:

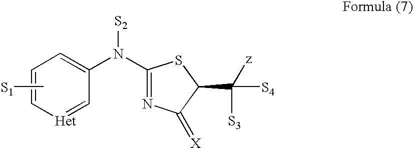

wherein S1-S4 represent independent substituents of the following type: Halogen (F, Cl, Br, or I); Hydroxyl (—OH); Sulfhydryl (—SH); Carboxylate (—COOH); Alkyl (C1-C4 carbons, straight chain, branched, or optionally containing unsaturation); Cycloalkyl (C1-C6 optionally containing unsaturation); Aryl including phenyl or heteroaryl containing from 1 to 4 N, O, and S atoms; Alkoxyl (—OR where R is defined as C1-C6 straight chain or branched alkyl group, optionally substituted with halogen, hydroxyl, sulfhydryl, carboxylate, aryl, heteroaryl, amino —NH₂, substituted amino —NR₂, or cycloamino groups containing one, two or three N atoms in a 5 or 6 membered ring); X is defined as O, S, N—R, N—OH, or N—NR₂; Het is defined as one or more N atoms, located at any position of the ring; Z is defined as —COOH, —PO₃H₂, SO₃H, tetrazole ring, sulfonamide, acyl sulfonamide group, —CONH₂, or —CONR₂.

Additional analogs include those where the central nitrogen atom is replaced with unsubstituted carbon atoms or carbon atoms containing one or two independent substituents where S2 and S6 are defined as above for S1-S4 or the central carbon atom bears the functionality X as described above:

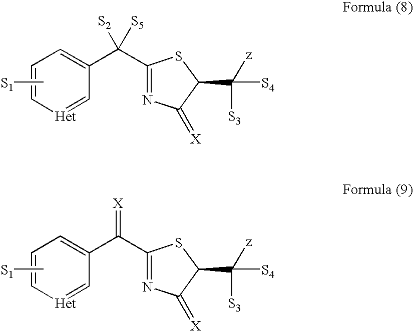

Compounds which have a short linker moiety as indicated are also expected to give the same inhibition of EGFR:

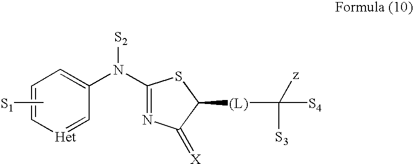

wherein L is defined as a linker consisting of 1-4 linearly connected atoms including C, N, O, and S. In the case of C and S, the oxidation state of the atom may have one or two oxygens attached by either a single or double bond. In the case of C or N, the atom may have one or two additional substituents independently selected from the group S1-S6 defined above.

Additionally, compounds of different stereochemical composition including racemates and enantiomeric isomers are also expected to have utility as EGFR inhibitors:

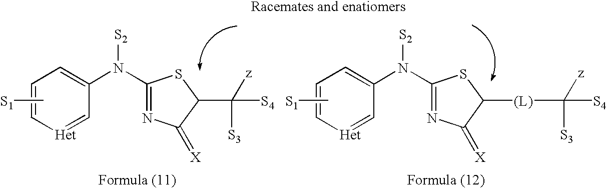

wherein S1-S4, X, Het, and Z are define as above.

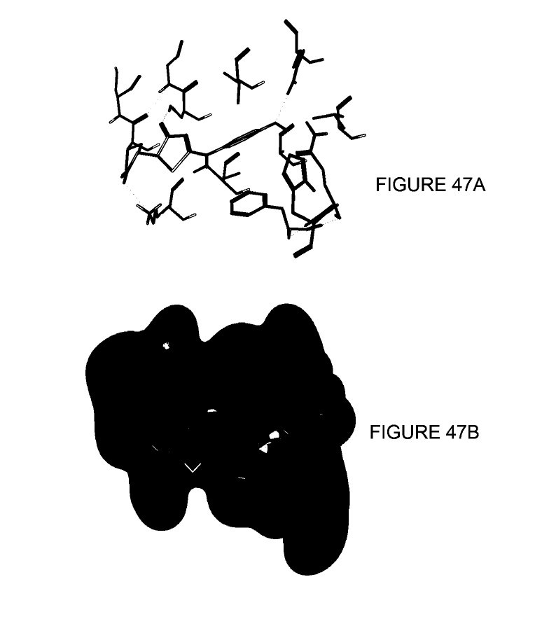

In one embodiment, the inhibitor of the binding of epidermal growth factor (EGF) to epidermal growth factor receptor (EGFR) is AD4-1038 (({2-[(4-Hydroxy-phenyl)-methyl-amino]-4-oxo-4,5-dihydro-thiazol-5-yl}-acetic acid; Formula: C₁₂H₁₂N₂O₄S; Molecular weight: 280.30) (see e.g., Example 8). An exemplary depiction of the binding of AD4-1038 to EGFR is shown in FIG. 47. The structure of AD4-1038 is as follows:

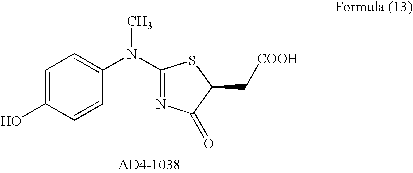

At a concentration of 25 μM of AD4-1038, binding of EGF to EGFR is inhibited by 70.7% (see e.g., Example 6).

AD4-1020

AD4-1020 is identified as an inhibitor of epidermal growth factor binding to its receptor (see e.g., Example 10). Such compounds have utility as treatments in oncology. Analogs and derivatives of AD4-1020 are expected to have the same inhibitory effect and utility. A pharmacophore model, Pharm1_gly54_asp58, was designed using information from the 1YY9 protein crystal structure to design a pharmacophore model (see e.g., Example 4). The Pharm1_gly54_asp58 model was utilized to identify small molecules which bind to EGFR (SEQ ID NO: 1). The site on the EGFR protein is recognized by amino acid residues GLY-54 to ASP-58 of the antibody Cetuximab (SEQ ID NO: 5 and SEQ ID NO:6) (Erbitux). Pharm1_gly54_asp58 is modeled after residues GLY-54 to ASP-58 and designed as a tool to identify small molecules which have features and components of the antibody cetuximab. Specifically this region is defined as the H2 CDR of the antibody heavy chain of cetuximab. Features and components of these amino acid residues of cetuximab were used to create a pharmacophore model. From this pharmacophore model is derived the following compound:

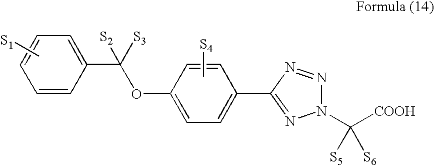

wherein S1-S6 represent independent substituents of the following type: Halogen (F, Cl, Br, I); Hydroxyl (—OH); Sulfhydryl (—SH); Carboxylate (—COOH); Alkyl (C1-C4 carbons, straight chain, branched or optionally containing unsaturation); Cycloalkyl (C1-C6 optionally containing unsaturation); Aryl including phenyl or heteroaryl containing from 1 to 4 N, O, and S atoms; or Alkoxyl (—OR where R is defined as C1-C6 straight chain or branched alkyl group, optionally substituted with halogen, hydroxyl, sulfhydryl, carboxylate, aryl, heteroaryl, amino —NH₂, substituted amino —NR₂, or cycloamino groups containing one, two or three N atoms in a 5 or 6 membered ring).

Additional analogs include those where one or both of the phenyl rings is replaced by a heterocyclic ring, wherein X is defined as O, S, N—R, N—OH, or N—NR₂; Het is defined as one or more N atoms, located at any position of the ring; and Z is defined as —COOH, —PO₃H₂; SO₃H, tetrazole ring, sulfonamide, acyl sulfonamide, —CONH₂, or —CONR₂.

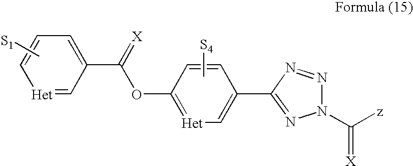

Additional analogs include those where Compounds which have a short linker moiety as indicated are also expected to give the same inhibition of EGFR, where L is defined as a linker consisting of 1-4 linearly connected atoms including C, N, O and S. In the case of C and S, the oxidation state of the atom may have one or two oxygens attached by either a single or double bond. In the case of C or N, the atom may have one or two additional substituents independently selected from the group S1-S6 defined above.

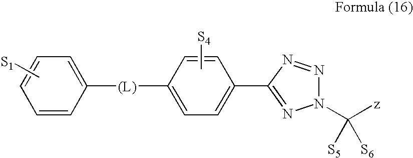

Additional analogs include compounds in which the tetrazole ring is replaced with an alternative 5-membered heterocyclic ring as indicated

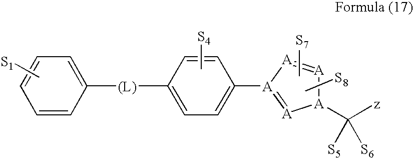

wherein A is an atom independently selected from a group including C, N, O, and S. In the case of C and S, the oxidation state of the atom may have one or two oxygens attached by either a single or double bond. In the case of C or N, the atom may have one or two additional substituents independently selected from the group S1-S6 defined above.

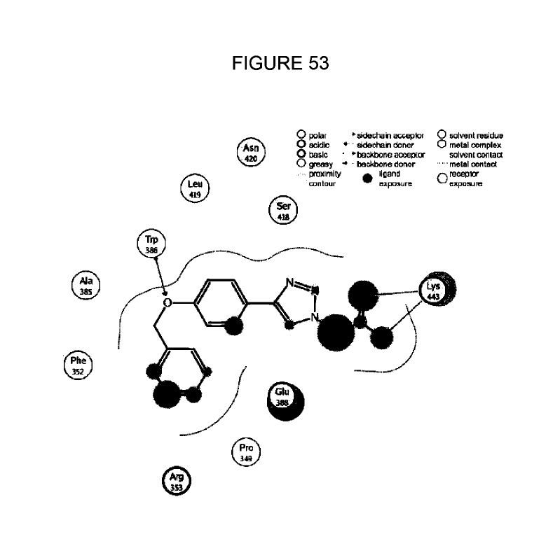

In one embodiment, the inhibitor of the binding of epidermal growth factor (EGF) to epidermal growth factor receptor (EGFR) is AD4-1020 (({5-[4-(benzyloxy)phenyl]-2H-tetrazol-2-yl}acetic acid); Formula: C₁₆H₁₄N₄O₃; Molecular weight: 310.31) (see e.g., Example 10). An exemplary depiction of the binding of AD4-1020 to EGFR is shown in FIG. 53. The structure of AD4-1020 is as follows:

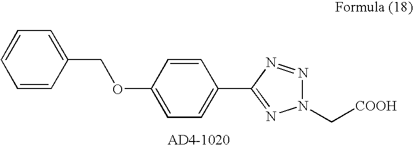

At a concentration of 25 μM of AD4-1020, binding of EGF to EGFR is inhibited by 47.8% (see e.g., Example 6).

AD4-1132

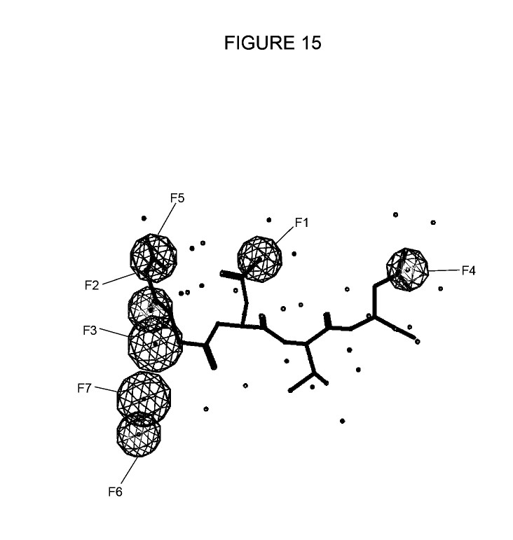

AD4-1132 is identified as an inhibitor of epidermal growth factor binding to its receptor (see e.g., Example 11). Such compounds have utility as treatments in oncology. Analogs and derivatives of AD4-1132 are expected to have the same inhibitory effect and utility. A pharmacophore model, Pharm23_gly54_asp58, was designed using information from the 1YY9 protein crystal structure to design a pharmacophore model (see e.g., Example 4; Table 17; FIG. 15). The Pharm23_gly54_asp58 model was utilized to identify small molecules which bind to EGFR (SEQ ID NO: 1). The site on the EGFR protein is recognized by amino acid residues GLY-54 to ASP-58 of the antibody Cetuximab (SEQ ID NO: 5 and SEQ ID NO:6) (Erbitux). Pharm23_gly54_asp58 is modeled after residues GLY-54 to ASP-58 and designed as a tool to identify small molecules which have features and components of the antibody cetuximab. Features and components of these amino acid residues of cetuximab were used to create a pharmacophore model. From this pharmacophore model is derived the following compound:

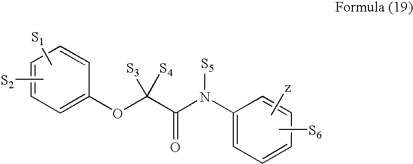

wherein S1-S6 represent independent substituents of the following type: Halogen (F, Cl, Br, I); Hydroxyl (—OH); Sulfhydryl (—SH); Carboxylate (—COOH); Alkyl (C1-C4 carbons, straight chain, branched or optionally containing unsaturation); Cycloalkyl (C1-C6 optionally containing unsaturation); Aryl including phenyl or heteroaryl containing from 1 to 4 N, O, and S atoms; or Alkoxyl (—OR where R is defined as C1-C6 straight chain or branched alkyl group, optionally substituted with halogen, hydroxyl, sulfhydryl, carboxylate, aryl, heteroaryl, amino —NH₂, substituted amino —NR₂, or cycloamino groups containing one, two or three N atoms in a 5 or 6 membered ring) and Z is defined as —COOH, —PO₃H₂; SO₃H, tetrazole ring, sulfonamide or acyl sulfonamide group, —CONH₂ or —CONR₂.

Additional analogs include those where the phenolic ether oxygen is replaced by atom of type Y, wherein Y is defined as CH₂, O, S, N—R, N—OH, or N—NR₂. In the case of C and S, the oxidation state of the atom may have one or two oxygens attached by either a single or double bond. In the case of C or N, the atom may have one or two additional substituents independently selected from the group S1-S6 defined above; and one or both phenyl rings is optionally replaced by a heterocyclic ring; wherein Het is defined as one or more N atoms, located at any position of the ring:

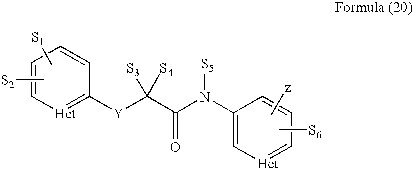

Additional analogs include those where compounds which have a short linker moiety as indicated are also expected to give the same inhibition of EGFR, where L is defined as a linker consisting of 1-4 linearly connected atoms including C, N, O and S, as indicated:

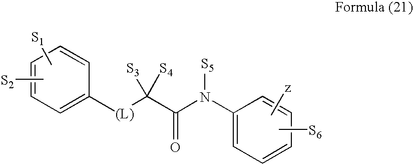

Additional analogs include compounds in which the amide nitrogen is replaced with an alternative group A, and the amide carbonyl is optionally replaced by the group X, as indicated:

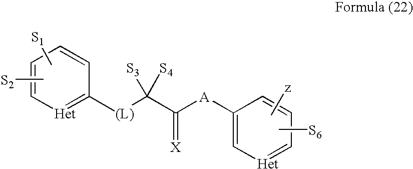

wherein A is an atom independently selected from a group including CH₂, N, O, and S. In the case of C and S, the oxidation state of the atom may have one or two oxygens attached by either a single or double bond. In the case of C or N, the atom may have one or two additional substituents independently selected from the group S1-S6 defined above, and X is defined as H₂, O, S, N—R, N—OH, or N—NR₂.

Additional analogs involve the juxtaposition of groups A and C═X as found in, but not limited to, the case of a retro-amide as indicated:

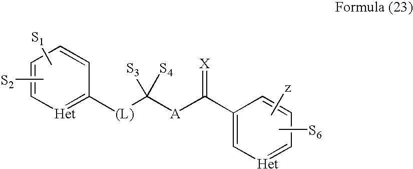

In one embodiment, the inhibitor of the binding of epidermal growth factor (EGF) to epidermal growth factor receptor (EGFR) is AD4-1132 ((2-{[(2,4-dimethylphenoxy)acetyl]amino}-5-hydroxybenzoic acid); Formula: C₁₇H₁₇NO₅; Molecular weight: 315.32) (see e.g., Example 11). The structure of AD4-1132 is as follows:

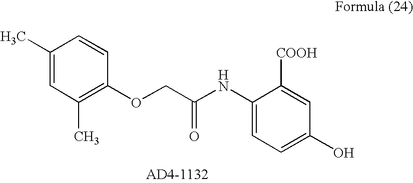

At a concentration of 25 μM of AD4-1132, binding of EGF to EGFR is inhibited by 59.6% (see e.g., Example 6).

AD4-1142

AD4-1142 is identified as an inhibitor of epidermal growth factor binding to its receptor (see e.g., Example 12). Such compounds have utility as treatments in oncology. Analogs and derivatives of AD4-1142 are expected to have the same inhibitory effect and utility. A pharmacophore model, Pharm23_gly54_asp58, was designed using information from the 1YY9 protein crystal structure to design a pharmacophore model (see e.g., Example 4). The Pharm23_gly54_asp58 model was utilized to identify small molecules which bind to EGFR (SEQ ID NO: 1). The site on the EGFR protein is recognized by amino acid residues GLY-54 to ASP-58 of the antibody Cetuximab (SEQ ID NO: 5 and SEQ ID NO:6) (Erbitux). Pharm23_gly54_asp58 is modeled after residues GLY-54 to ASP-58 and designed as a tool to identify small molecules which have features and components of the antibody cetuximab. Specifically this region is defined as the H2 CDR of the antibody heavy chain of cetuximab. Features and components of these amino acid residues of cetuximab were used to create a pharmacophore model. From this pharmacophore model is derived the following compound:

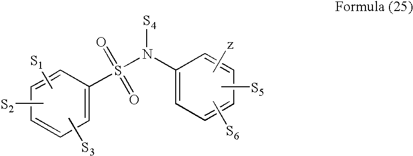

wherein S1-S6 represent independent substituents of the following type: Hydrogen (—H); Halogen (F, Cl, Br, I); Hydroxyl (—OH); Sulfhydryl (—SH); Carboxylate (—COOH); Alkyl (C1-C4 carbons, straight chain, branched or optionally containing unsaturation); Cycloalkyl (C1-C6 optionally containing unsaturation); Aryl including phenyl or heteroaryl rings containing from 1 to 4 N, O, and S atoms; or Alkoxyl (—OR where R is defined as C1-C6 straight chain or branched alkyl group, optionally substituted with halogen, hydroxyl, sulfhydryl, carboxylate, aryl, heteroaryl, amino —NH₂, substituted amino —NR₂, or cycloamino groups containing one, two or three N atoms in a 5 or 6 membered ring) and Z is defined as —COOH, —PO₃H₂; SO₃H, tetrazole ring, sulfonamide, acyl sulfonamide, —CONH₂, or —CONR₂.

Additional analogs include those where the sulfonamide NH is optionally replaced by atom of type Y, wherein Y is defined as CH₂, O, S, N—R, N—OH, or N—NR₂; and one or both phenyl rings is optionally replaced by a heterocyclic ring; wherein Het is defined as one or two N atoms located at any position of the ring.

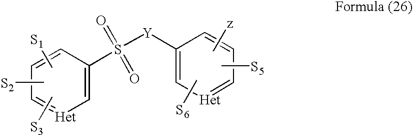

Additional analogs include those where Compounds which have a short linker moiety as indicated are also expected to give the same inhibition of EGFR, where L is defined as a linker consisting of 1-4 linearly connected atoms including C, N, O and S. In the case of C and S, the oxidation state of the atom may have one or two oxygens attached by either a single or double bond. In the case of C or N, the atom may have one or two additional substituents independently selected from the group S1-S6 defined above.

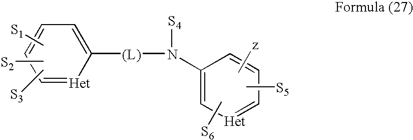

Additional analogs include compounds in which aromatic groups are connected by groups A, and Y as indicated; including analogs where groups A and Y are optionally connected by single, double and triple bonds,

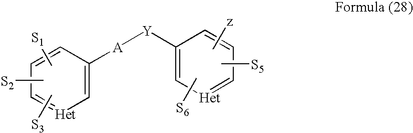

wherein Y is define as above and A is an atom independently selected from a group including CH₂, N, O, and S. In the case of C and S, the oxidation state of the atom may have one or two oxygens attached by either a single or double bond. In the case of C or N, the atom may have one or two additional substituents independently selected from the group S1-S6 defined above.

Additional analogs involve the juxtaposition of groups A and Y as indicated, including analogs where groups A and Y are optionally connected by single, double and triple bonds,

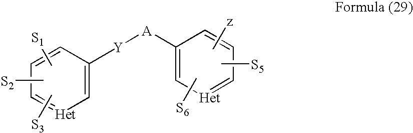

In one embodiment, the inhibitor of the binding of epidermal growth factor (EGF) to epidermal growth factor receptor (EGFR) is AD4-1142 ((5-{[(4-ethylphenyl)sulfonyl]amino}-2-hydroxybenzoic acid); Formula: C₁₅H₁₅NO₅S; Molecular weight: 321.35) (see e.g., Example 12). The structure of AD4-1142 is as follows:

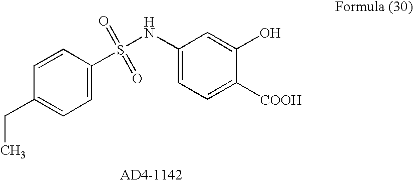

At a concentration of 25 μM of AD4-1142, binding of EGF to EGFR is inhibited by 49.8% (see e.g., Example 6).

Pharmaceutical Formulations

Embodiments of the compositions of the invention include pharmaceutical formulations of the various compounds described herein. The compounds described herein can be formulated by any conventional manner using one or more pharmaceutically acceptable carriers and/or excipients as described in, for example, Remington's Pharmaceutical Sciences (A. R. Gennaro, Ed.), 21 st edition, ISBN: 0781746736 (2005). Such formulations will contain a therapeutically effective amount of the agent, preferably in purified form, together with a suitable amount of carrier so as to provide the form for proper administration to the subject. The formulation should suit the mode of administration. The agents of use with the current invention can be formulated by known methods for administration to a subject using several routes which include, but are not limited to, parenteral, pulmonary, oral, topical, intradermal, intramuscular, intraperitoneal, intravenous, subcutaneous, intranasal, epidural, ophthalmic, buccal, and rectal. The individual agents may also be administered in combination with one or more additional agents of the present invention and/or together with other biologically active or biologically inert agents. Such biologically active or inert agents may be in fluid or mechanical communication with the agent(s) or attached to the agent(s) by ionic, covalent, Van der Waals, hydrophobic, hydrophillic or other physical forces.

Controlled-release (or sustained-release) preparations may be formulated to extend the activity of the agent and reduce dosage frequency. Controlled-release preparations can also be used to effect the time of onset of action or other characteristics, such as blood levels of the agent, and consequently affect the occurrence of side effects.

When used in the methods of the invention, a therapeutically effective amount of one of the agents described herein can be employed in pure form or, where such forms exist, in pharmaceutically acceptable salt form and with or without a pharmaceutically acceptable excipient. For example, the agents of the invention can be administered, at a reasonable benefit/risk ratio applicable in a sufficient amount sufficient to inhibit the target biomolecule for which the compound is specific for the treatment or prophylaxis of a disease, disorder, or condition associated with the target biomolecule.

Toxicity and therapeutic efficacy of such compounds, and pharmaceutical formulations thereof, can be determined by standard pharmaceutical procedures in cell cultures and/or experimental animals for determining the LD₅₀ (the dose lethal to 50% of the population) and the ED₅₀, (the dose therapeutically effective in 50% of the population). The dose ratio between toxic and therapeutic effects is the therapeutic index that can be expressed as the ratio LD₅₀/ED₅₀, where large therapeutic indices are preferred.

The amount of a compound of the invention that may be combined with a pharmaceutically acceptable carrier to produce a single dosage form will vary depending upon the host treated and the particular mode of administration. It will be appreciated by those skilled in the art that the unit content of agent contained in an individual dose of each dosage form need not in itself constitute a therapeutically effective amount, as the necessary therapeutically effective amount could be reached by administration of a number of individual doses. Agent administration can occur as a single event or over a time course of treatment. For example, an agent can be administered daily, weekly, bi-weekly, or monthly. For some conditions, treatment could extend from several weeks to several months or even a year or more.

The specific therapeutically effective dose level for any particular subject will depend upon a variety of factors including the condition being treated and the severity of the condition; activity of the specific agent employed; the specific composition employed; the age, body weight, general health, sex and diet of the patient; the time of administration; the route of administration; the rate of excretion of the specific agent employed; the duration of the treatment; drugs used in combination or coincidental with the specific agent employed and like factors well known in the medical arts. It will be understood by a skilled practitioner that the total daily usage of the compounds for use in the present invention will be decided by the attending physician within the scope of sound medical judgment.

Compounds of the invention that inhibit the target biomolecule can also be used in combination with other therapeutic modalities. Thus, in addition to the therapies described herein, one may also provide to the subject other therapies known to be efficacious for particular conditions linked to the target biomolecule.

Having described the invention in detail, it will be apparent that modifications, variations, and equivalent embodiments are possible without departing the scope of the invention defined in the appended claims. Furthermore, it should be appreciated that all examples in the present disclosure are provided as non-limiting examples.

### EXAMPLES

The following non-limiting examples are provided to further illustrate the present invention. It should be appreciated by those of skill in the art that the techniques disclosed in the examples that follow represent approaches the inventors have found function well in the practice of the invention, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the invention.

### Example 1

### Vascular Endothelial Growth Factor

The following example is directed toward the generation of one or more pharmacophores based at least in part upon antibodies raised against a target molecule, in this example human vascular endothelial growth factor (VEGF-A) (SEQ ID NO: 2). In short, a human vascular endothelial growth factor (VEGF-A) is presented to a number of animals (for example a group of genetically dissimilar mice). The inoculation and repeated presentation of the VEGF-A results in the animals raising a variety of IgG antibodies (polyclonal high affinity antibodies), against the molecule. These antibodies differ across the animals as each has a distinct genetic potential for antibody production (different combinations of possible CDRs). The variation in the antibodies results in them binding to the VEGF-A molecule at different surface areas of the molecule. It is expected that at least one of the antibodies binds to the active region of the VEGF-A molecule.

By way of clarification, the VEGF family currently comprises seven members: VEGF-A, VEGF-B, VEGF-C, VEGF-D, VEGF-E, VEGF-F, and P1GF. All members have a common VEGF homology domain that includes a cystine knot motif, with eight invariant cysteine residues involved in inter- and intramolecular disulfide bonds at one end of a conserved central four-stranded beta-sheet within each monomer, which dimerize in an antiparallel, side-by-side orientation.

MAB Generation; Crystallization, X-Ray Diffraction; Spatial Position

A VEGF molecule bound to an affinity-matured antibody (the Fab fragment thereof) has been previously crystallized and published by Chen, et al. in the RCSB database as 1CZ8. More particularly, their crystallization data includes regions V and W, which are the members of the VEGF dimer, and regions L, H, X, and Y which represent the antibody light and heavy chains of the Fab molecule. (More particularly, the L and H regions comprise one of the branches of the Fab molecule, including both the variable and constant regions of each chain. Similarly, X and Y are the light and heavy chains of the other branch of the Fab molecule.)

By geometrically analyzing the spatial arrangement of the more than eight thousand non-hydrogen atoms of the crystalline structure of VEGF-A, those atoms of one structure that are within a specified distance of atoms of another structure can be determined. This filter determines, by a straightforward geometric comparison across all possible combinations, the peptides that are in association across the two molecules. Using a maximum separation of four Angstroms, those atoms of the heavy variable chain (H) that are within such a short range of atoms of the W component of the VEGF dimer are determined, which are most likely to be the ones in the CDRs of the Fab fragment.

In the present instance, this analysis revealed that the following amino acids of the H region of the antibody fragment and the W region of the VEGF molecule included side chain atoms that were within 4 angstroms of one another (set forth in a table with the total number of side chain atoms between the two that are within that range):

TABLE 1

| | | H chain peptides (amino acid and sequence number) | | | | | | | | | | | | | | | |
|---|---|---|---|---|---|---|---|---|---|---|---|---|---|---|---|---|---|
| 4 Å | | Thr | His | Tyr | Trp | Asn | Thr | Tyr | Thr | Tyr | Pro | Tyr | Tyr | Tyr | Gly | Ser | Trp |
| separation | | 30 | 31 | 32 | 50 | 52 | 53 | 54 | 59 | 99 | 100 | 101 | 102 | 103 | 104 | 106 | 108 |
| W | Tyr | | | | | | | | | | | | | | | 3 | |
| chain | 45 | | | | | | | | | | | | | | | | |
| | Gln | | | | | | | | | | | 5 | | | | | |
| | 79 | | | | | | | | | | | | | | | | |
| | Ile 80 | | | | | | | | | | | | | | | | |
| | Arg | | | | | | | | | | | | 9 | 1 | 3 | 2 | |
| | 82 | | | | | | | | | | | | | | | | |
| | Ile 83 | | | | | 2 | | | | | | | | | | | |
| | His | | | | 1 | | | | 2 | | | | | | | | |
| | 86 | | | | | | | | | | | | | | | | |
| | Gln | | | | 4 | | | | | 3 | | | | | | | 2 |
| | 87 | | | | | | | | | | | | | | | | |
| | Gly | | | | 1 | | | | | | | | | | | | 2 |
| | 88 | | | | | | | | | | | | | | | | |
| | Gln | 1 | 1 | | 2 | 10 | 3 | 1 | | 2 | | | | | | | |
| | 89 | | | | | | | | | | | | | | | | |
| | His | | | 1 | | | | | | | 4 | | 9 | | | 5 | 8 |
| | 90 | | | | | | | | | | | | | | | | |
| | Ile 91 | | 5 | 5 | | | | | | | | 1 | 2 | | | | |
| | Gly | | | | | | | | | | | | 2 | | | | |
| | 92 | | | | | | | | | | | | | | | | |
| | Glu | | | | | | | | | | | 8 | 1 | 5 | | | |
| | 93 | | | | | | | | | | | | | | | | |
| | Met | | | | | | | | | | | | 2 | | | | |
| | 94 | | | | | | | | | | | | | | | | |

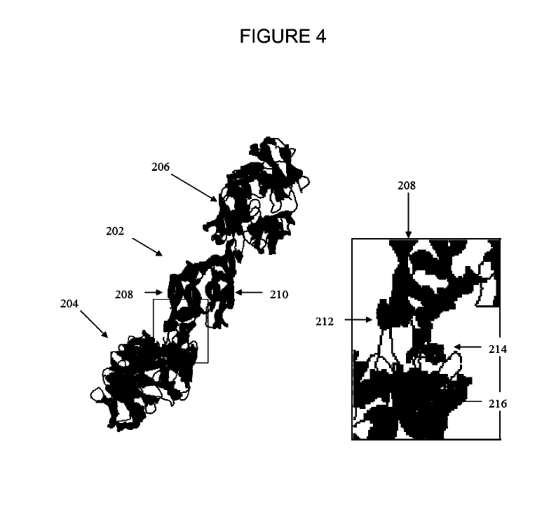

This analysis further confirms that the antibody binding to the target protein has been identified correctly as it involves an interaction between the CDRs of the antibody, as the location of the CDRs on the variable heavy chain include peptides in the 30-33, 50-55, and 100-110 ranges. More specifically, as is shown in FIG. 4, which is a computer simulation of the VEGF 202 bound to the Fabs 204,206, the target protein is constructed of dimerized molecules W 208 and V 210. Despite the fact that this antibody is an affinity-matured version, it can be seen in the focus box to the right of the entire molecular model that the interaction between the lower Fab 204 is limited to two of the CDRs 212,214 of the variable region 216 of the heavy chain.

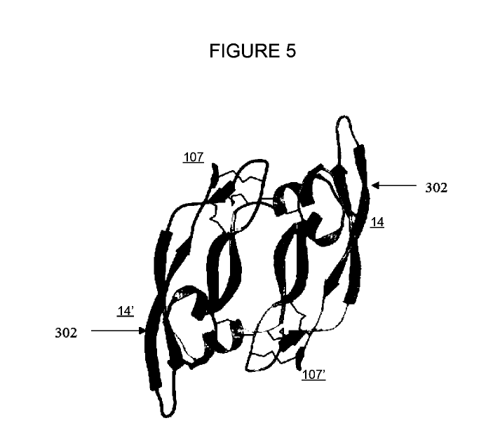

FIG. 5 shows the ribbon model of the dimerized VEGF molecule, and confirms that the range of peptides 302 that are engaged by the antibody are in the range of 80 to 100, which is again by the analysis summarized in the table above.

According to the Chen, et al., in vitro cell-based assays show that this affinity-matured antibody yielded significant potency for inhibition of VEGF-dependent cell proliferation. An aspect of the present invention is to recognize the possibility of using the structure of the binding interface of the antibody as a guide to the generation of a synthetic lead molecule.

This level of accuracy is helpful to confirm that the peptides believed to be involved in binding to the target are actually members of the CDRs, and the closeness of the atoms are not simply an artifact of the crystallization process. In order to isolate the most vital atoms involved in the binding, however, to use as a model for the synthesis of a lead molecule, it is necessary to narrow the number of atom-to-atom interactions down to only those few that are most closely associated. This can be achieved by reducing the acceptable separation in the filter to 3 angstroms. Table 2 below shows the results of this more focused analysis.

TABLE 2

| Heavy chain | | | VEGF | | | |
|---|---|---|---|---|---|---|
| (peptide, #, atom) | | | (peptide, #, atom) | | | Dist (Å) |
| Ile | 80 | O | Tyr | 102 | OH | 2.47 Å |
| Gln | 87 | O | Tyr | 99 | OH | 2.59 Å |
| Gln | 89 | NE2 | Thr | 30 | O | 2.78 Å |
| His | 90 | ND1 | Pro | 100 | O | 2.67 Å |
| His | 90 | NE2 | Ser | 106 | OG | 2.59 Å |
| Glu | 93 | OE2 | Tyr | 101 | OH | 2.92 Å |

Looking more closely at the relative positioning of these twelve atoms, and more particularly to the six atoms of the antibody that bind with the atoms of the target, a lead molecule can be constructed. The table below includes the relative distances of each of these atoms from one another.

TABLE 3

| | | | Heavy Chain Atoms in closest contact | | | | | |
|---|---|---|---|---|---|---|---|---|
| | | | with the VEGF target molecule | | | | | |
| | | | THR | TYR | PRO | TYR | TYR | SER |
| | | | 30 | 99 | 100 | 101 | 102 | 106 |
| | | | O | OH | O | OH | OH | OG |
| THR | 30 | O | 0.00 | 13.07 | 10.77 | 13.90 | 12.39 | 13.18 |
| TYR | 99 | OH | 13.07 | 0.00 | 9.65 | 18.53 | 14.69 | 9.70 |
| PRO | 100 | O | 10.77 | 9.65 | *0.00 | *9.15 | *8.35 | *6.99 |
| TYR | 101 | OH | 13.90 | 18.53 | *9.15 | *0.00 | *7.89 | *12.17 |
| TYR | 102 | OH | 12.39 | 14.69 | *8.35 | *7.89 | *0.00 | *6.09 |
| SER | 106 | OG | 13.18 | 9.70 | *6.99 | *12.17 | *6.09 | *0.00 |

As can be seen above, the atoms that are most closely associated in the binding region of the target and the antibody are Oxygen atoms. Nitrogen atoms are also highly prevalent among these high affinity sites. Oxygen and nitrogen atoms are often interchangeable when a hydrogen acceptor or donor is necessary.

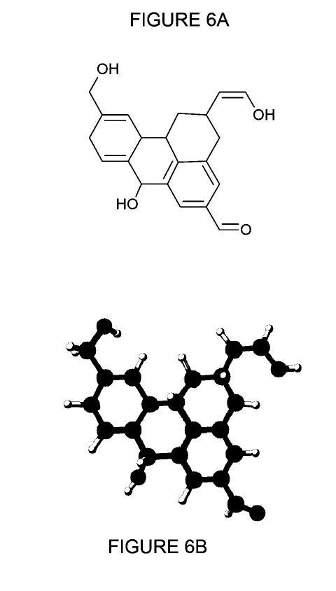

The asterisked numbers in Table 3, showing distances among the identified atoms of the binding region, represent an ideal subgroup of the atoms involved in binding that are close enough together to be structured into a lead molecule. The 4 oxygen atoms of the proline 100, tyrosines 101 and 102, and the serine 106 are close enough (<13 Å apart) that a suitable molecule can be constructed that has drug-like size. FIGS. 6A and 6B show a lead molecule structure that meets these criteria. The tables below show (i) the separation of the atoms in a reasonable conformation of the lead molecule, and (ii) the difference between the positions of the atoms in the lead as compared with the data from the x-ray diffraction analysis of the crystallized antibody.

TABLE 4

Key Atoms
Relative Positions
in the Lead Molecule

| | | PRO | TYR | TYR | SER |
|---|---|---|---|---|---|
| | | O | OH | OH | O |
| PRO | O | 0.00 | 9.15 | 8.35 | 6.99 |
| TYR | OH | 9.15 | 0.00 | 7.89 | 12.17 |
| TYR | OH | 8.35 | 7.89 | 0.00 | 6.09 |
| SER | O | 6.99 | 12.17 | 6.09 | 0.00 |

TABLE 5

Difference from
Antibody to Lead Molecule

| | | PRO | TYR | TYR | SER |
|---|---|---|---|---|---|
| | | O | OH | OH | O |
| PRO | O | 0.00 | 0.29 | 0.42 | 0.08 |
| TYR | OH | 0.29 | 0.00 | 0.27 | 0.38 |
| TYR | OH | 0.42 | 0.27 | 0.00 | 0.03 |
| SER | O | 0.08 | 0.38 | 0.03 | 0.00 |

As the tables above show, the proposed lead molecule, generated by the methods of the present invention, provides key atoms that are positioned with an average of 0.18 Å deviation (and no more than 0.42 Å deviation) from their relative locations in the antibody's binding tip.

As described above, the four “rules of five” state that a candidate drug-like compound should have at least three of the following characteristics (i) a weight less than 500 Daltons, (ii) have a log of P greater than 5; (iii) have at no more than 5 hydrogen bond donors (expressed as the sum of OH and NH groups); and (iv) have no more than 10 hydrogen bond acceptors (the sum of N and O atoms). The presently described lead, C₂₁H₂₀O₄, has the following characteristics: (i) a molecular weight of 336; (ii) 2 hydrogen bond donors; and (iii) 4 hydrogen bond acceptors.

### Example 2

### Influenza Glycoprotein

Another desirable target might be a protein associated with a viral infection, for example the hemagglutinin. Hemagglutinin is an antigenic glycoprotein found on the surface of the influenza viruses and is responsible for binding the virus to the cell that is being infected.

Millions of people in the United States (some estimates range as high as 10% to 20% of U.S. residents) are infected with influenza each year, despite an aggressive media campaign by vaccine manufacturers, medical associations, and government organizations concerned with public health. Most people who get influenza will recover in one to two weeks, but others will develop life-threatening complications (such as pneumonia). While typically considered by many to be simply a bad version of a cold, influenza can be deadly, especially for the weak, old or chronically ill. An average of about 36,000 people per year in the United States die from influenza, and 114,000 per year are admitted to a hospital as a result of influenza. According to estimates by the World Health Organization, between 250,000 and 500,000 die from influenza infection each year worldwide. Some flu pandemics have killed millions of people, including the most deadly outbreak which killed upwards of 50 million people between 1918 and 1920.

Failure of many patients to avail themselves of the vaccines against the flu may be the result of the fact that mutations in the glycoproteins found in the viral coat makes annual vaccinations a requirement to fully protect an individual from the latest version of the virus. A medication capable of blocking the ability of the virus to bind to the cells of the host, even if only partially effective, would dramatically enhance the likelihood that the infected individual's immune system would defeat the infection before clinically significant symptoms appear. If the medication were to be made available after the beginning of such symptoms, a reduction in the severity and duration of the infection are possible as well.

Fleury, et al., have published the results of their crystallization of hemagglutinin complexed with a neutralizing antibody. The data from their x-ray crystallization efforts are provided in the protein data bank and were analyzed by the inventor hereof in a manner similar to that disclosed hereinabove with respect to VEGF (see Example 1).

More specifically, a geometric analysis of the spatial arrangement of the more than eight thousand non-hydrogen atoms of the hemagglutinin complexed with a neutralizing antibody crystalline structure was conducted to determine those atoms of the antibody fragments (heavy and light variable chains) that are close enough to the target protein to be part of the binding to the hemagglutinin. The filters used, including straightforward geometric methods, confirmed that the variable heavy chain CDR regions, and in particular peptides within the CDR1 and CDR3 (peptides 26-32 and 99-102 in particular, according to the Kabat and Wu numbering) were the ones that provided the tightest binding of the antibody to the hemagglutinin protein.

Using a maximum separation of four Angstroms, those atoms within these CDRs were determined, as well as the specific atoms within the target glycoprotein that are engaged with one another. These are provided in the following table:

TABLE 6

| Heavy chain | | | | Hemagglutinin | | | |
|---|---|---|---|---|---|---|---|
| (peptide, #, atom) | | | | (peptide, #, atom) | | | Dist (Å) |
| | Gly | 26 | O | Lys | 92 | O | 3.13 Å |
| | Ser | 28 | CB | Asp | 271 | OD2 | 3.05 Å |
| | Thr | 31 | OG1 | Asp | 271 | O | 2.82 Å |
| | Tyr | 32 | OH | Arg | 90 | NE | 3.39 Å |
| | Tyr | 32 | OH | Asp | 60 | OD2 | 2.97 Å |
| | Arg | 94 | NH1 | Ile | 62 | CD1 | 3.24 Å |
| | Arg | 94 | NH2 | Asp | 63 | OD1 | 2.74 Å |
| | Trp | 100 | NE1 | Asp | 63 | OD2 | 3.07 Å |
| | Phe | 100A | CD2 | His | 75 | O | 2.78 Å |

Looking more closely at the relative positioning of these eighteen atoms, and more particularly to the nine atoms of the antibody that bind with the atoms of the target, a lead molecule can be constructed. The table below includes the relative distances of each of these atoms from one another.

TABLE 7

| | | | Heavy Chain Atoms in closest | | | | | | | |
|---|---|---|---|---|---|---|---|---|---|---|
| | | | contact with the Hemagglutinin molecule | | | | | | | |
| | | | GLY | SER | THR | TYR | ARG | ARG | TRP | PHE |
| | | | 26 | 28 | 31 | 32 | 94 | 94 | 100 | 100A |
| | | | O | CB | OG1 | OH | NH1 | NH2 | NE1 | CD1 |
| GLY | 26 | O | 0.00 | 7.31 | 10.05 | 8.92 | 5.36 | 7.05 | 12.61 | 11.00 |
| SER | 28 | CB | 7.31 | 0.00 | 3.92 | 6.96 | 8.00 | 10.17 | 16.81 | 13.34 |
| THR | 31 | OG1 | 10.05 | 3.92 | 0.00 | 5.22 | 8.72 | 10.42 | 16.64 | 12.47 |
| TYR | 32 | OH | 8.92 | 6.96 | 5.22 | *0.00 | *6.12 | *6.92 | *11.64 | *7.37 |
| ARG | 94 | NH1 | 5.36 | 8.00 | 8.72 | *6.12 | *0.00 | *2.31 | *10.17 | *7.33 |
| ARG | 94 | NH2 | 7.05 | 10.17 | 10.42 | *6.92 | *2.31 | *0.00 | *8.31 | *5.70 |
| TRP | 100 | NE1 | 12.61 | 16.81 | 16.64 | *11.64 | *10.17 | *8.31 | *0.00 | *4.71 |
| PHE | 100A | CD1 | 11.00 | 13.34 | 12.47 | *7.37 | *7.33 | *5.70 | *4.71 | *0.00 |

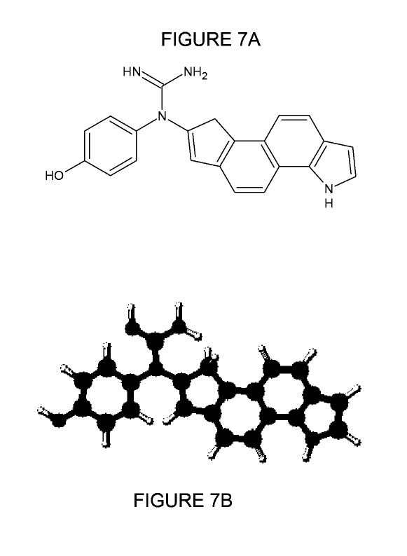

The asterisked numbers represent an ideal subgroup of the atoms involved in binding that are close enough together to be structured into a lead molecule. More specifically, a drug-like molecule typically has a span of between 8-15 Å and a molecular weight of less than 500 Daltons. The 5 atoms of the tyrosine 32, arginine 94, tryptophan 100, and phenylalanine 100A are close enough (<12 Å apart) that a suitable molecule can be constructed that has drug-like size. FIGS. 7A and 7B show a lead molecule structure that meets these criteria. The tables below show (i) the separation of the atoms in a reasonable conformation of the lead molecule, and (ii) the difference between the positions of the atoms in the lead as compared with the data from the x-ray diffraction analysis of the crystallized antibody.

TABLE 8

| | | Key Atoms Relative Positions | | | | |
|---|---|---|---|---|---|---|
| | | in the Lead Molecule | | | | |
| | | OH | NH | NH | N | C |
| | | TYR | ARG | ARG | TRP | PHE |
| OH | TYR | 0.00 | 5.79 | 7.44 | 12.19 | 8.03 |
| NH | ARG | 5.79 | 0.00 | 2.24 | 10.08 | 6.91 |
| NH | ARG | 7.44 | 2.24 | 0.00 | 8.26 | 5.83 |
| N | TRP | 12.19 | 10.08 | 8.26 | 0.00 | 4.17 |

| | | | | | |
|---|---|---|---|---|---|
| “Resonant | 8.03 | 6.91 | 5.83 | 4.17 | 0.00 |
| Ring” C | | | | | |

TABLE 9

| | | Difference from | | | | |
|---|---|---|---|---|---|---|
| | | Antibody to Lead Molecule | | | | |
| | | OH | NH | NH | N | C |
| | | TYR | ARG | ARG | TRP | PHE |
| OH | TYR | 0.00 | 0.33 | 0.52 | 0.55 | 0.66 |
| NH | ARG | 0.33 | 0.00 | 0.07 | 0.09 | 0.42 |
| NH | ARG | 0.52 | 0.07 | 0.00 | 0.05 | 0.13 |
| N | TRP | 0.55 | 0.09 | 0.05 | 0.00 | 0.54 |

| | | | | | |
|---|---|---|---|---|---|
| “Resonant | 0.66 | 0.42 | 0.13 | 0.54 | 0.00 |
| Ring” C | | | | | |

As the tables above show, the proposed lead molecule, generated by the methods of the present invention, provides key atoms that are positioned with an average of 0.33 Å deviation (and no more than 0.66 Å deviation) from their relative locations in the antibody's binding tip.

As introduced earlier, the four “rules of five” state that a candidate drug-like compound should have at least three of the following characteristics (i) a weight less than 500 Daltons, (ii) have a log of P less than 5; (iii) have at no more than 5 hydrogen bond donors (expressed as the sum of OH and NH groups); and (iv) have no more than 10 hydrogen bond acceptors (the sum of N and O atoms). The presently described lead, C₂₂H₁₈N_{4O}, has the following characteristics: (i) a molecular weight of 354; (ii) 3 hydrogen bond donors; and (iii) 5 hydrogen bond acceptors.

### Example 3

### Angiogenin

Angiogenesis (sprouting of new capillary vessels from pre-existing vasculature) is a critical aspect of development in the fetus and in children, as their circulatory system expands during growth. In adults angiogenesis is required during the normal tissue repair, and for the remodeling of the female reproductive organs (ovulation and placental development). Certain pathological conditions, however, such as tumor growth and diabetic retinopathy, also require angiogenesis. A known factor involved in angiogenesis is angiogenin, which is a single polypeptide chain of 123 amino acids.

Angiogenin is one of the normal cytokines that is commandeered by cancer to assist in its rapid growth. In this case, tumor cells secrete angiogenin in order to recruit greater blood flow to the tumor. It would, therefore, be of great value to find a drug that could inhibit the production of, or the activity of angiogenin.

Chavali, et al., have published the results of their crystallization of angiogenin complexed with a neutralizing antibody. The data from their x-ray crystallization efforts are provided in the protein data bank and have been analyzed by the inventor hereof in a manner similar to that disclosed hereinabove with respect to both VEGF and Hemagglutinin.

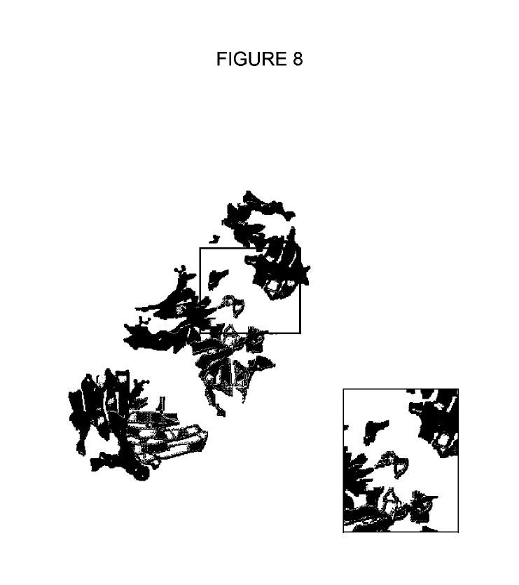

More specifically, a geometric analysis of the spatial arrangement of the non-hydrogen atoms of the crystalline structure was conducted to determine those atoms of the antibody fragments (heavy and light variable chains) that are close enough to the angiogenin molecule to be part of the binding to it. The filters used, including straightforward geometric methods, confirmed what can be seen in FIG. 8, which is that both the variable light and heavy chains, and in particular peptides within the CDR1 of the light chain, and CDRs 2 and 3 of the heavy chain were the ones that provided the tightest binding of the antibody to the angiogenin.

Using a maximum separation of four Angstroms, those atoms within these CDRs were determined, as well as the specific atoms within the target glycoprotein that are engaged with one another. These are provided in the following table:

TABLE 10

| Antibody | | | | Hemagglutinin | | | |
|---|---|---|---|---|---|---|---|
| (chain, peptide, #, atom) | | | | (peptide, #, atom) | | | Dist (Å) |
| Tyr | L | 30B | OH | Leu | 35 | O | 3.31 Å |
| Asn | L | 30A | ND2 | Ser | 37 | O | 2.70 Å |
| Tyr | L | 30B | OH | Ser | 37 | O | 3.51 Å |
| Tyr | L | 30B | OH | Pro | 38 | O | 2.61 Å |
| Tyr | H | 98 | CD1 | Cys | 39 | O | 3.56 Å |
| Tyr | H | 100B | OH | Cys | 39 | O | 2.78 Å |
| Ser | H | 52A | OG | Gly | 85 | O | 3.15 Å |
| Thr | H | 33 | OG1 | Gly | 86 | O | 3.31 Å |
| Tyr | H | 58 | OH | Trp | 89 | O | 2.91 Å |
| Ser | L | 90 | O | Trp | 89 | NE1 | 3.01 Å |
| Asn | H | 56 | OD1 | Pro | 91 | O | 3.28 Å |

Looking more closely at the relative positioning of these eighteen atoms, and more particularly to the nine atoms of the antibody that bind with the atoms of the target, two separate potential target sights are identified on the angiogenin. This means that (as shown in Tables 11 and 12) two separate lead molecules can be constructed to bind with the angiogenin. The tables below include the relative distances of each of these atoms in each group from one another.

TABLE 11

Atoms in closest contact
with the first site on
Angiogenin molecule

| | | | TYR | ASN | TYR | TYR |
|---|---|---|---|---|---|---|
| | | | 30B | 30A | 98 | 100B |
| | | | OH | N | C | OH |
| TYR | 30B | OH | 0.00 | 3.36 | 7.03 | 8.27 |
| ASN | 30A | N | 3.36 | 0.00 | 9.37 | 10.89 |
| TYR | 98 | C | 7.03 | 9.37 | 0.00 | 3.90 |
| TYR | 100B | OH | 8.27 | 10.89 | 3.90 | 0.00 |

TABLE 12

Atoms in closest contact
with the second site on
Angiogenin molecule

| | | | THR | TYR | SER | ASN |
|---|---|---|---|---|---|---|
| | | | 33 | 58 | 90 | 56 |
| | | | O | OH | O | O |
| THR | 33 | O | 0.00 | 7.18 | 11.28 | 9.38 |
| TYR | 58 | OH | 7.18 | 0.00 | 9.96 | 3.68 |
| SER | 90 | O | 11.28 | 9.96 | 0.00 | 13.47 |
| ASN | 56 | O | 9.38 | 3.68 | 13.47 | 0.00 |

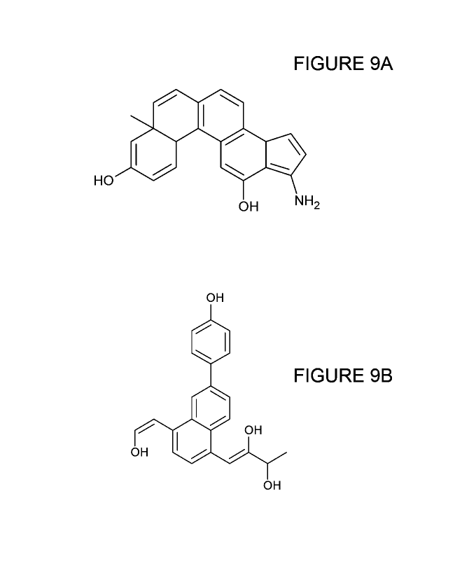

As stated previously, a drug-like molecule typically has a span of between 8-15 Å and a molecular weight of less than 500 Daltons. In Table 11, it can be seen that the OH of tyrosine 30B, the nitrogen of asparagine 30A, the resonant carbon CD2 of tyrosine 98, and the OH of tyrosine 100B are close enough (<11 Å apart) that a suitable molecule can be constructed that has drug-like size. Similarly, with respect to Table 12, the oxygen of threonine 33, the OH of tyrosine 58, and the oxygens of serine 90 and asparagine 56 are close enough (<14 Å apart) that another suitable molecule can be constructed. FIGS. 9A and 9B show two lead molecule structures that meets the criteria for the first and second regions of the angiogenin molecules respectively.

The tables below show (i) the separation of the atoms in a reasonable conformation of the first lead molecule, and (ii) the difference between the positions of the atoms in the first lead as compared with the data from the x-ray diffraction analysis of the crystallized antibody.

TABLE 13

Key Atoms Relative
Positions in the 1^{st}
Lead Molecule

| | | OH | N | C | OH |
|---|---|---|---|---|---|
| | | TYR | ASN | TYR | TYR |
| OH | TYR | 0.00 | 3.51 | 7.03 | 8.06 |
| N | ASN | 3.51 | 0.00 | 9.41 | 10.75 |
| “Resonant | | 7.03 | 9.41 | 0.00 | 3.95 |
| Ring” C | | | | | |
| OH | TYR | 8.06 | 10.75 | 3.95 | 0.00 |

TABLE 14

Key Atoms Relative
Positions in the 1^{st}
Lead Molecule

| | | OH | N | C | OH |
|---|---|---|---|---|---|
| | | TYR | ASN | TYR | TYR |
| OH | TYR | 0.00 | 3.51 | 7.03 | 8.06 |
| N | ASN | 3.51 | 0.00 | 9.41 | 10.75 |
| “Resonant | | 7.03 | 9.41 | 0.00 | 3.95 |
| Ring” C | | | | | |
| OH | TYR | 8.06 | 10.75 | 3.95 | 0.00 |

Similarly tables below show (i) the separation of the atoms in a reasonable conformation of the second lead molecule, and (ii) the difference between the positions of the atoms in the second lead as compared with the data from the x-ray diffraction analysis of the crystallized antibody.

TABLE 15

Key Atoms Relative
Positions in the 2^{nd}
Lead Molecule

| | | O | OH | O | O |
|---|---|---|---|---|---|
| | | THR | TYR | SER | ASN |
| O | THR | 0.00 | 7.17 | 11.15 | 9.23 |
| OH | TYR | 7.17 | 0.00 | 9.89 | 3.65 |
| O | SER | 11.15 | 9.89 | 0.00 | 13.47 |
| O | ASN | 9.23 | 3.65 | 13.47 | 0.00 |

TABLE 16

Difference from
Antibody to the 2^{nd}
Lead Molecule

| | | O | OH | O | O |
|---|---|---|---|---|---|
| | | THR | TYR | SER | ASN |
| O | THR | 0.00 | 0.01 | 0.13 | 0.15 |
| OH | TYR | 0.01 | 0.00 | 0.07 | 0.03 |
| O | SER | 0.13 | 0.07 | 0.00 | 0.00 |
| O | ASN | 0.15 | 0.03 | 0.00 | 0.00 |

As the tables above show, the proposed lead molecule, generated by the methods of the present invention, provides key atoms that are positioned with an average of 0.05 Å deviation (and no more than 0.15 Å deviation) from their relative locations in the antibody's binding tip.

As introduced earlier, the four “rules of five” state that a candidate drug-like compound should have at least three of the following characteristics (i) a weight less than 500 Daltons, (ii) have a log of P less than 5; (iii) have at no more than 5 hydrogen bond donors (expressed as the sum of OH and NH groups); and (iv) have no more than 10 hydrogen bond acceptors (the sum of N and O atoms). The first lead candidate, C₂₂H₁₉NO₂, has the following characteristics: (i) a molecular weight of 329; (ii) 4 hydrogen bond donors; and (iii) 4 hydrogen bond acceptors. The second lead candidate, C₂₂H₂₀O₄, has the following characteristics: (i) a molecular weight of 348; (ii) 4 hydrogen bond donors; and (iii) 4 hydrogen bond acceptors.

### Example 4

### Generation of Pharmacophores for Target Inhibition

The following example describes analysis of target protein-antibody crystal structure complexes and generation of pharmacophores for identifying molecules which inhibit EGFR, HER2, and ErbB2 binding.

The protein crystal structure of cetuximab complexed to EGFR is reported by Ferguson et al. (Cancer Cell, 2005, 7, 301-311) and the crystallographic data is deposited in the Protein Data Bank as PDB code 1YY9. Structural information which defines the position of the atoms of Cetuximab (SEQ ID NO: 5 and SEQ ID NO:6) was utilized to construct a pharmacophore model used to identify small molecules having corresponding atoms in similar positions. Small molecules having similar features to the antibody can demonstrate similar biological activity and thus have similar therapeutic utility.

The pharmacophore feature generation and pharmacophore virtual screening module of the Molecular Operating Environment (MOE) software from Chemical Computing Group (CCG) (Montreal, Quebec, Canada) was used in the pharmacophore definitions described below. MOE's pharmacophore applications use a general notion of a pharmacophore being a set of structural features in a ligand that are directly related to the ligand's recognition at a receptor site and thus its biological activity.

In MOE, pharmacophoric structural features are represented by labeled points in space. Each ligand is assigned an annotation, which is a set of structural features that may contribute to the ligand's pharmacophore. A database of annotated ligands can be searched with a query that represents a pharmacophore hypothesis. The result of such a search is a set of matches that align the pharmacophoric features of the query to the pharmacophoric features present in the ligands of the searched database. The of MOE software suite provides for interactive modifications (positions, radii, as well as other characteristics of the pharmacophoric query can be interactively adjusted); systematic matching (all possible matches of the ligand and the query are systematically examined); partial matching (the search algorithm is capable of finding ligands that match only a portion of the query); and volume filtering (the query can be focused by adding restrictions on the shape of the matched ligands in the form of a set of volumes).

The pharmacophore features of this example were generated using the Pharmacophore Query Editor in MOE. All hydrogen bond donor features are spheres of 1.2 Angstroms in radius and are colored purple. All hydrogen bond acceptor features are spheres of 1.2 Angstroms in radius and are colored cyan. All aromatic features are spheres of 1.2 Angstroms in radius and are colored green. All combined acceptor-anion pharmacophore features are spheres of 1.2 Angstroms in radius and are colored grey. All combined donor-acceptor features are spheres of 1.2 Angstroms in radius and are colored pink. All combined donor-cation features are spheres of 1.2 Angstroms and are colored red. All donor, acceptor, aromatic, combined acid-anion, and combined donor-acceptor directionality features are spheres of 1.5 Angstroms in radius and colored dark grey for donors, dark cyan for acceptors, dark green for aromatics, dark cyan for combined acid-anions, and dark grey for combined donor-acceptors. A feature that is marked essential in the pharmacophore query must be contained in the ligand in order for that ligand to be a hit.

All of the pharmacophore features were derived from the corresponding donor, acceptor, aromatic and acid moieties of the corresponding antibody in complex with its receptor (e.g., cetuximab complexed with EGFr, pdb accession number 1YY9) taken from crystal structures deposited in the protein databank (PDB:1YY9) with two exceptions. In some cases two methods provided by the MOE software are used to place pharmacophore features. These are explained below.

The Contact statistics calculated, using the 3D atomic coordinates of a receptor, preferred locations for hydrophobic and hydrophilic ligand atoms using statistical methods. Using this method hydrophobic-aromatic and H-bonding features were placed, as noted in the individual pharmacophore definitions.

The MultiFragment Search essentially places a relatively large number of copies of a fragment (e.g., 200 copies of ethane) into a receptor's active site. The fragments are placed randomly around the active site atoms and are assumed not to interact with each other; no regard is paid to fragment overlap. Next, a special energy minimization protocol is used to refine the initial placement: the receptor atoms feel the average forces of the fragments, while each fragment feels the full force of the receptor but not of the other fragments. Using this technique it was possible to place hydrophobic, H-bond donors, acceptors and anions and cations in favorable positions within the receptors for use as MOE pharmacophore features.

Excluded volumes were generated for the pharmacophores defined below except when indicated. These were derived from the position of the receptor atoms near the antibody binding site. Excluded volumes are positions in space where ligand atoms must be excluded in order to avoid bumping into the receptor. They were generated in MOE by selecting the receptor residues within 5 Angstroms from the antibody and selecting “union” from the pharmacophore query editor in MOE.

In the Individual Pharmacophore Definitions described below, abbreviations were as follows: F=pharmacophore feature; Donor=Don, Acceptor=Acc, Anion=Ani, Cation=Cat, Acceptor and Anion=Acc&Ani, Donor and Cation=Don&Cat, Donor and Acceptor=Don&Acc, Aromatic=Aro, Hydrophobe=Hyd.

EGFR Complexed with Antibody Cetuximab (1YY9.pdb)

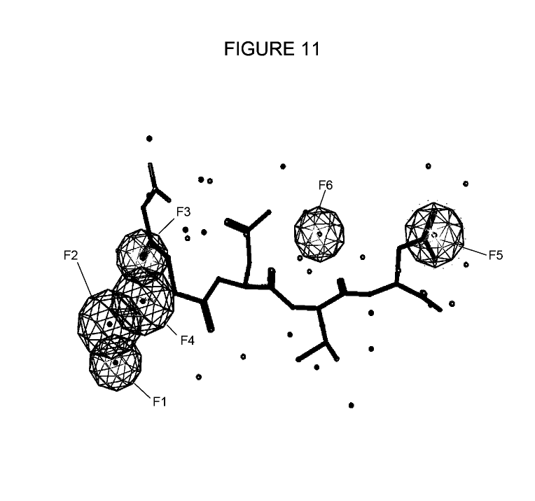

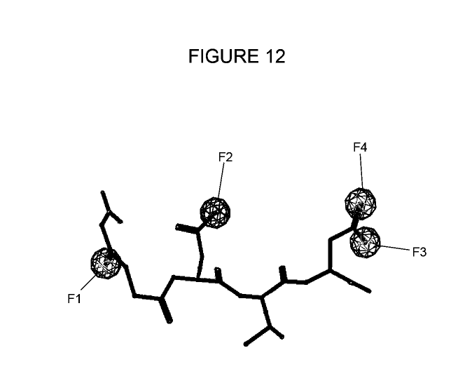

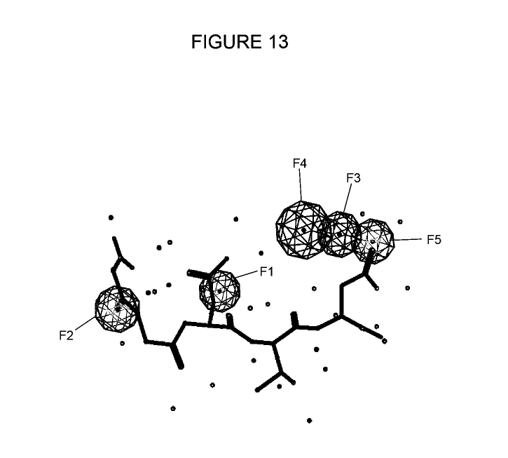

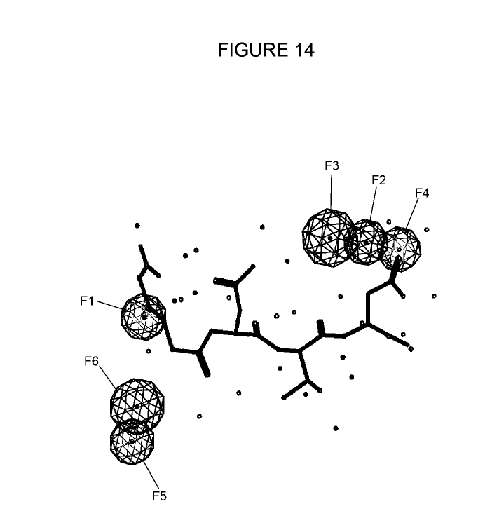

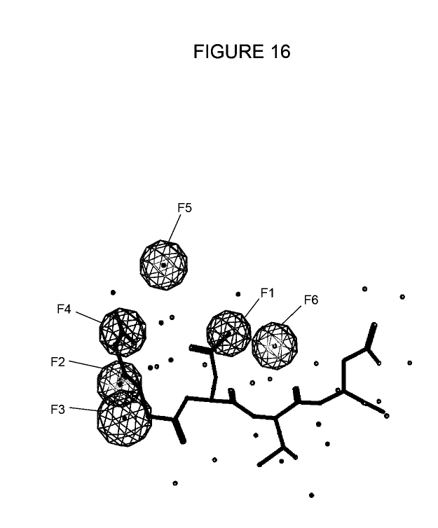

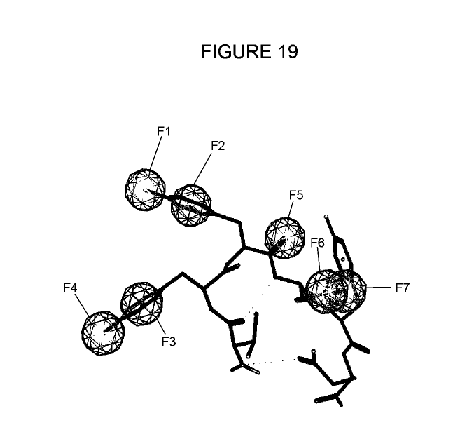

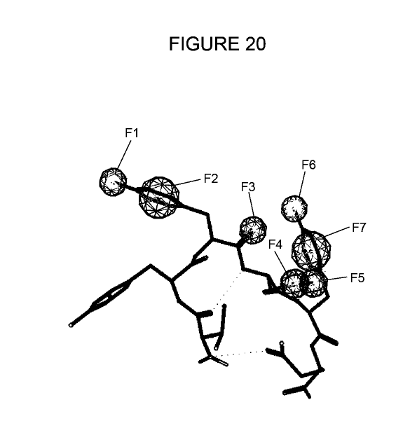

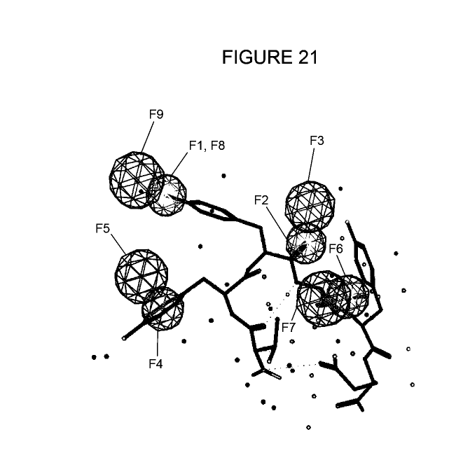

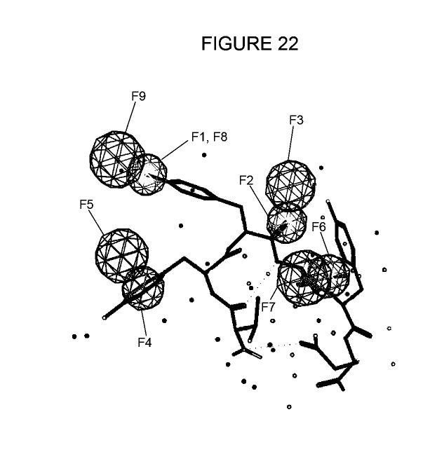

The crystal (1YY9.pdb) of protein EGFR (SEQ ID NO: 1) complexed with antibody Cetuximab (SEQ ID NO: 5 and SEQ ID NO:6) was analyzed according to the procedures described above. Results showed that two sets of residues of the antibody cetuximab make contact with the receptor. These are Gly54-Asp58 and Thr100-Glu105. Since these sets of residues of the antibody are not in close proximity to each other, they were used to generate two groups of pharmacophore models, for regions gly54_asp58 and thr100_glu105, described below in Table 17 and depicted in FIGS. 11-22.

TABLE 17
Pharmacophores of 1YY9.pdb crystal of protein EGFR complexed with antibody cetuximab

| Pharmacophore | F | comment |
|---|---|---|
| 1_gly54_asp58 | F1 Aro | Derived from hydrophobic contact statistics, favorable coulombic |
| Partial match, | | interaction with guanidine of Arg353 of the receptor. |
| ligand must | F2 Aro2 | Directionality of F1 with respect to guanidine of Arg353. |
| match at least 5 | F3 | Derived from Gly54 backbone carbonyl of the antibody |
| pharmacophore | Acc&Ani | cetuximab. Acceptor accepts an H-bond from or Anion forms a |
| features. | | salt bridge to guanidine of receptor Arg353. |
| | F4 Acc2 | Directionality of F3 with respect to guanidine of Arg353. |
| | F5 | Derived from Asp58 side chain carboxylate. Acceptor accepts an |
| | Acc&Ani | H-bond from or Anion forms a salt bridge to NH₃⁺ of Lys 443 side |
| | | chain of the receptor. |
| | F6 Acc | Derived from hydrophilic contact statistics. Accepts an H-bond |
| | | from side chain OH of Ser448 of the receptor. |
| | V1 | Excluded volumes (not shown for clarity). |
| 11_gly54_asp58 | F1 Acc | Derived from antibody Gly54 backbone carbonyl accepts an H- |
| Full match | | bond from guanidine of receptor Arg353. |
| query. All | F2 Don | Derived from antibody Asn56 side chain NH2 donating an H- |
| pharmacophore | | bond to receptor Ser448. |
| feature spheres | F3 and | Derived from antibody Asp58 side chain carboxylate accepting |
| have a 0.8 | F4 | an H-bond from or forming a salt bridge to NH₃⁺ of receptor Lys |
| Angstrom | Acc&Ani | 443. |
| radius. | | |
| | V1 | Excluded volumes (not shown for clarity). |
| 21_gly54_asp58 | F1 Aro | Derived from MFSS (see above). Forms a favorable |
| Partial match, | | hydrophobic interaction with pyrrolidine ring of receptor Pro349. |
| ligand must | F2 | Derived from antibody Gly54 backbone carbonyl. Acceptor |
| match at least 4 | Acc&Ani | accepts an H-bond from or Anion forms a salt bridge to |
| pharmacophore | | guanidine of receptor Arg353. |
| features. | F3 Don | Derived from hydrophilic contact statistics. Donates an H-bond |
| | | to side chain OH of receptor Ser418. |
| | F4 Don2 | Directionality of F3 with respect to OH of Ser418 |
| | F5 Acc | Derived from hydrophilic contact statistics. Accepts an H-bond |
| | | from receptor Lys443. This feature is marked essential. |
| | V1 | Excluded volumes (not shown for clarity). |
| 22_gly54_asp58 | F1 | Derived from Gly54 backbone carbonyl of the antibody |
| Partial match, | Acc&Ani | cetuximab. Acceptor accepts an H-bond from or Anion forms a |
| ligand must | | salt bridge to guanidine of receptor Arg353. |
| match at least 5 | F2 Don | Derived from hydrophilic contact statistics. Donates an H-bond |
| pharmacophore | | to side chain OH of receptor Ser418. |
| features | F3 Don2 | Directionality of F3 with respect to OH of Ser418. |
| | F4 Acc | Derived from hydrophilic contact statistics. Accepts an H-bond |
| | | from receptor Lys443. |
| | F5 Aro | Derived from hydrophobic contact statistics, favorable coulombic |
| | | interaction with guanidine of Arg353 of the receptor. |
| | F6 Aro2 | Directionality of F1 with respect to guanidine of Arg353. |
| 23_gly54_asp58 | F1 Don | Derived from antibody side chain NH2 of Asn56 forms an H- |
| Partial match, | | bond with receptor Ser418 side chain OH. |
| ligand must | F2 | Derived from Gly54 backbone carbonyl of the antibody |
| match at least 5 | Acc&Ani | cetuximab. Acceptor accepts an H-bond from or Anion forms a |
| pharmacophore | | salt bridge to guanidine of receptor Arg353. |
| features | F3 Acc2 | Directionality of F3 with respect to guanidine of Arg353. |
| | F4 | Derived from antibody Asp58 side chain carboxylate accepting |
| | Acc&Ani | an H-bond from or forming a salt bridge to NH₃⁺ of receptor Lys |
| | | 443. |
| | F5 Don | Derived from antibody Gly54 backbone NH forming an H-bond |
| | | with side chain carbonyl of receptor Gln384. |
| | F6 Aro | Derived from hydrophobic contact statistics, favorable coulombic |
| | | interaction with guanidine of Arg353 of the receptor. This feature |
| | | is marked essential. |
| | F7 Aro2 | Directionality of F6 with respect to guanidine of Arg353. |
| 24_gly54_asp58 | F1 Don | Derived from antibody side chain NH2 of Asn56 forms an H- |
| Partial match, | | bond with receptor Ser418 side chain OH. |
| ligand must | F2 | Derived from Gly54 backbone carbonyl of the antibody |
| match at least 5 | Acc&Ani | cetuximab. Acceptor accepts an H-bond from or Anion forms a |
| pharmacophore | | salt bridge to guanidine of receptor Arg353. |
| features | F3 Acc2 | Directionality of F3 with respect to guanidine of Arg353. |
| | F4 Don | Derived from antibody Gly54 backbone NH forming an H-bond |
| | | with side chain carbonyl of receptor Gln384. |
| | F5 Aro | Derived from hydrophobic contact statistics, favorable coulombic |
| | | interaction with side chain CONH2 of receptor Gln384. |
| | F6 | Derived from hydrophilic contact statistics. Acceptor or anion |
| | Acc&Ani | accepts an H-bond from OH side chain of receptor Ser418. |
| | V1 | Excluded volumes |
| 1_thr100_glu105 | F1 Acc | Derived from the backbone carbonyl of the antibody Tyr102 |
| Partial match, | | accepting an H-bond from the OH side chain of the receptor |
| ligand must | | Ser440. |
| match at least 6 | F2 Acc2 | Directionality of F1 with respect to the OH of Ser440. |
| pharmacophore | F3 Aro | Derived from side chain phenol ring of the antibody Tyr101 |
| features | | forming a favorable coulombic interaction with the imidazole side |
| | | chain of the receptor His408. |
| | F4 Aro2 | Directionality of F3 with respect to the imidazole of His409. |
| | F5 Don | Derived from the side chain OH of the antibody Tyr102 donating |
| | | an H-bond to the side chain carbonyl of the receptor Gln408. |
| | F6 Aro | Derived from the phenol side chain of antibody Tyr102 forming a |
| | | favorable hydrophobic interaction with the side chain of receptor |
| | | Val417. |
| | F7 Don2 | Directionality of F5 with respect to the side chain carbonyl of |
| | | Gln408. |
| | F8 | Derived from the side chain carboxylate of antibody Asp103. |
| | Acc&Ani | Acceptor accepts an H-bond from or Anion forms a salt bridge to |
| | | NH₃⁺ of receptor Lys465. This feature is marked essential. |
| | V1 | Excluded volumes |
| 2_thr100_glu105 | | This pharmacophore query is the same as 1_thr100_glu105 with |
| Partial match, | | the exception that F8 Acc&Ani is not marked essential. |
| ligand must | | |
| match at least 7 | | |
| pharmacophore | | |
| features | | |
| 3_thr100_glu105 | F1 | Derived from the side chain OH of antibody Tyr102. This OH |
| Partial match, | Don&Acc | donates an H-bond to the side chain carbonyl of receptor |
| ligand must | | Gln408 and accepts an H-bond from the side chain NH2 of |
| match at least 5 | | receptor Gln384. |
| pharmacophore | F2 Aro | Derived from the side chain phenol ring of antibody Tyr102 |
| features | | forming a favorable hydrophobic interaction with the side chain |
| | | of receptor Val417. |
| | F3 Aro | Derived from side chain phenol ring of the antibody Tyr101 |
| | | forming a favorable coulombic interaction with the imidazole side |
| | | chain of the receptor His409. |
| | F4 | Derived from side chain OH of Tyr101. |
| | Don&Acc | |
| | F5 Acc | Derived from the backbone carbonyl of antibody Tyr102 |
| | | accepting an H-bond from the side chain OH of receptor Ser440. |
| | F6, F7 | Derived from the side chain carboxylate of antibody Asp103. |
| | Acc&Ani | Acceptor accepts an H-bond from or Anion forms a salt bridge to |
| | | NH₃⁺ of receptor Lys465. |
| | V1 | Excluded volume |
| 10_thr100_glu105 | F1 | Derived from the side chain OH of antibody Tyr102. This OH |
| Partial match, | Don&Acc | donates an H-bond to the side chain carbonyl of receptor |
| ligand must | | Gln408 and accepts an H-bond from the side chain NH2 of |
| match at least 5 | | receptor Gln384. |
| pharmacophore | F2 Aro | Derived from the side chain phenol ring of antibody Tyr102 |
| features. All | | forming a favorable hydrophobic interaction with the side chain |
| pharmacophores | | of receptor Val417. |
| spheres have a | F3 Acc | Derived from the backbone carbonyl of antibody Tyr102 |
| radius of 0.8 | | accepting an H-bond from the side chain OH of receptor Ser440. |
| Angstroms. | F4, F5 | Derived from the side chain carboxylate of antibody Asp103. |
| | Acc&Ani | Acceptor accepts an H-bond from or Anion forms a salt bridge to |
| | | NH₃⁺ of receptor Lys465. |
| | F6 | Derived from the side chain OH of antibody Tyr104 donating or |
| | Don&Acc | accepting an H-bond from the side chain OH of receptor Ser440. |
| | F7 Aro | Derived from side chain phenol ring of antibody Tyr104 forming |
| | | a favorable hydrophobic interaction with receptor Ser468. |
| 21_thr100_glu105 | F1 | Derived from the side chain OH of antibody Tyr102. This OH |
| Partial match, | Don&Acc | donates an H-bond to the side chain carbonyl of receptor |
| ligand must | | Gln408 and accepts an H-bond from the side chain NH2 of |
| match at least 7 | | receptor Gln384. |
| pharmacophore | F2 Acc | Derived from the backbone carbonyl of antibody Tyr102 |
| features. | | accepting an H-bond from the side chain OH of receptor Ser440. |
| | F3 Acc2 | Directionality of F2 with respect to the OH of Ser440. |
| | F4 Aro | Derived from side chain phenol ring of the antibody Tyr101 |
| | | forming a favorable coulombic interaction with the imidazole side |
| | | chain of the receptor His409. |
| | F5 Aro2 | Directionality of F4 with respect to the imidazole of His409. |
| | F6 | Derived from the side chain carboxylate of antibody Asp103. |
| | Acc&Ani | Acceptor accepts an H-bond from or Anion forms a salt bridge to |
| | | NH₃⁺ of receptor Lys465. |
| | F7 Acc2 | Directionality of F6 with respect to the side chain of Lys465. |
| | F8 Don | Derived from the side chain OH of antibody Tyr102. This OH |
| | | donates an H-bond to the side chain carbonyl of receptor |
| | | Gln408. This feature is marked essential. |
| | F9 Don2 | Directionality with respect to the side chain carbonyl of receptor |
| | | Gln408. |
| | V1 | Excluded volume |
| 22_thr100_glu105 | | This pharmacophore query is the same as 21_thr100_glu_105 |
| Partial match, | | with two exceptions: |
| ligand must | | F6 Aci&Ani is marked essential. |
| match at least 6 | | F8 Don is not marked essential. |
| pharmacophore | | |
| features. | | |

VEGF Complexed with Antibody Cetuximab (1CZ8)

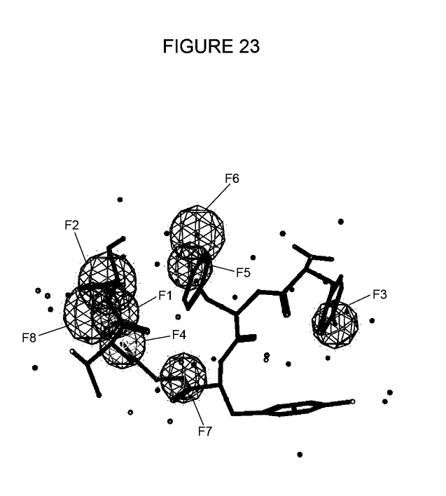

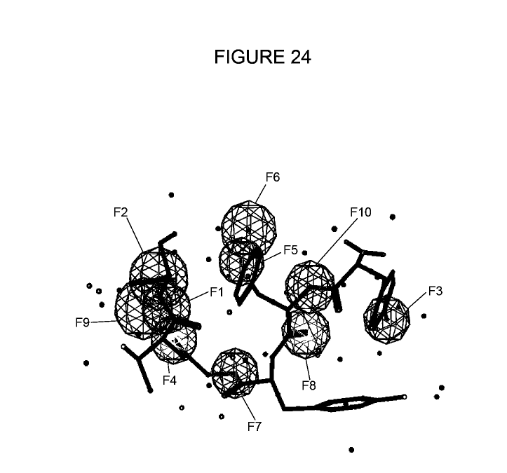

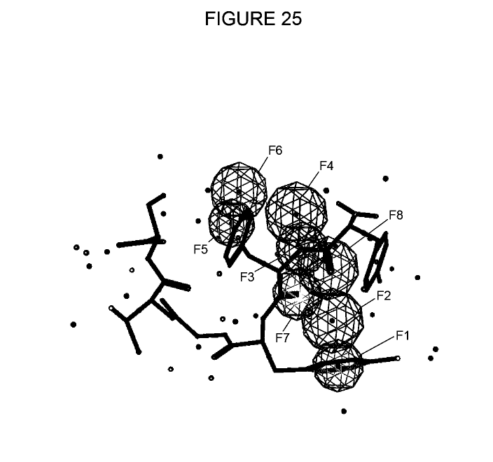

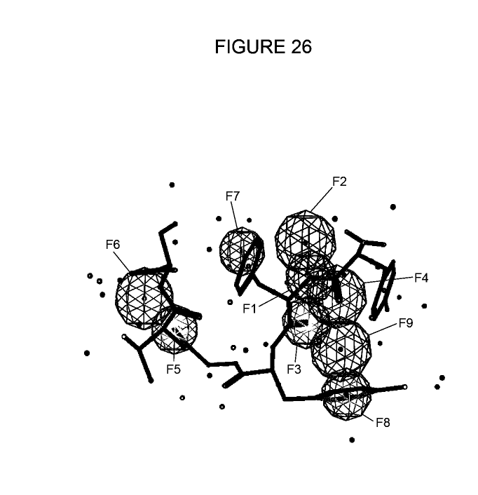

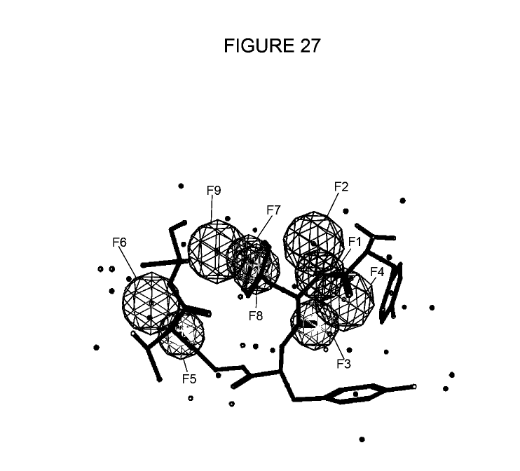

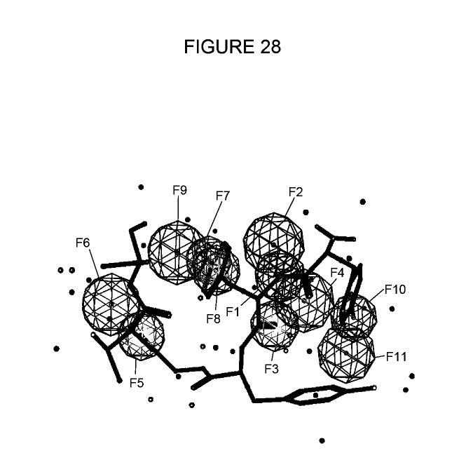

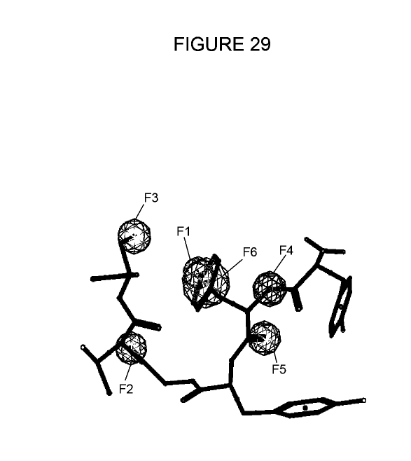

The crystal (1CZ8) of protein VEGF (SEQ ID NO: 2) complexed with antibody was analyzed according to the procedures described above. Results showed that one set of six residues of the antibody makes contact with the receptor. This is Tyr101-Ser106. This section of the antibody is used to generate the pharmacophore models described below in Table 18 and FIGS. 23-29.

TABLE 18
Pharmacophores of 1CZ8.pdb crystal of protein VEGF complexed with antibody

| Pharmacophore | F | comment |
|---|---|---|
| 1n | F1 Aro | Derived from hydrophobic contact statistics, favorable coulombic |
| Partial match, | | interaction with guanidine of Arg82 of the receptor |
| ligand must | F2 Aro2 | Directionality of F1 with respect to guanidine of Arg82. |
| match at least 6 | F3 Don | Derived from the side chain OH of antibody Tyr101. This OH |
| pharmacophore | | donates an H-bond to the side chain carboxylate of receptor |
| features. | | Glu93. |
| | F4 | Derived from Gly104 backbone carbonyl of the antibody. |
| | Acc&Ani | Acceptor accepts an H-bond from or Anion forms a salt bridge to |
| | | guanidine side chain of receptor Arg82. |
| | F5 Don | Derived from the side chain OH of antibody Tyr102. This OH |
| | | donates an H-bond to the backbone carbonyl of receptor Ile80. |
| | F6 Don2 | Directionality of F5 with respect to the backbone carbonyl Ile80. |
| | F7 Don | Derived from the backbone NH of antibody Gly104. This NH |
| | | donates an H-bond to the backbone carbonyl of receptor Glu93. |
| | F8 Acc2 | Directionality of F7 with respect to the backbone carbonyl Glu93. |
| | V1 | Excluded volume |
| 2n | F1 Aro | Derived from hydrophobic contact statistics, favorable coulombic |
| Partial match, | | interaction with guanidine of Arg82 of the receptor. |
| ligand must | F2 Aro2 | Directionality of F1 with respect to guanidine of Arg82. |
| match at least 7 | F3 Don | Derived from the side chain OH of antibody Tyr101. This OH |
| pharmacophore | | donates an H-bond to the side chain carboxylate of receptor |
| features. | | Glu93. This feature is marked ignored. |
| | F4 | Derived from Gly104 backbone carbonyl of the antibody. |
| | Acc&Ani | Acceptor accepts an H-bond from or Anion forms a salt bridge to |
| | | guanidine side chain of receptor Arg82. |
| | F5 Don | Derived from the side chain OH of antibody Tyr102. This OH |
| | | donates an H-bond to the backbone carbonyl of receptor Ile80. |
| | F6 Don2 | Directionality of F5 with respect to the backbone carbonyl Ile80. |
| | F7 Don | Derived from the backbone NH of antibody Gly104. This NH |
| | | donates an H-bond to the backbone carbonyl of receptor Glu93. |
| | F8 Acc2 | Directionality of F7 with respect to the backbone carbonyl Glu93. |
| | F9 Acc | Derived from backbone carbonyl of antibody Tyr102 accepting |
| | | an H-bond from the backbone NH of receptor Glu93. |
| | F10 Don | Derived from the backbone NH of antibody Tyr102. This NH |
| | | donates an H-bond to the backbone carbonyl of receptor Ile91. |
| | V1 | Excluded volume |
| 3n | F1 Aro | Derived from side chain phenol of Tyr103 forming a favorable |
| Partial match, | | hydrophobic interaction with the side chain of receptor Glu93. |
| ligand must | F2 Aro2 | Directionality of F1 with respect to the side chain of Glu93. |
| match at least 8 | F3 Don | Derived from the backbone NH of antibody Tyr102. This NH |
| pharmacophore | | donates an H-bond to the backbone carbonyl of receptor Ile91. |
| features. | F4 Don2 | Directionality of F3 with respect to the backbone carbonyl of |
| | | receptor Ile91. |
| | F5 Don | Derived from the side chain OH of antibody Tyr102. This OH |
| | | donates an H-bond to the backbone carbonyl of receptor Ile80. |
| | F6 Don2 | Directionality of F5 with respect to the backbone carbonyl of |
| | | receptor Ile80. |
| | F7 Acc | Derived from backbone carbonyl of Tyr102 accepting an H-bond |
| | | from the backbone NH of receptor Glu93. |
| | F8 Acc2 | Directionality of F7 with respect to the backbone NH of receptor |
| | | Glu93. |
| | V1 | Excluded volume |
| 4n | F1 Don | Derived from the backbone NH of antibody Tyr102. This NH |
| Partial match, | | donates an H-bond to the backbone carbonyl of receptor Ile91. |
| ligand must | F2 Don2 | Directionality of F1 with respect to the backbone carbonyl Ile91. |
| match at least 7 | F3 Acc | Derived from backbone carbonyl of antibody Tyr102 accepting |
| pharmacophore | | an H-bond from the backbone NH of receptor Glu93. |
| features. | F4 Acc2 | Directionality of F3 with respect to the backbone NH of receptor |
| | | Glu93. |
| | F5 | Derived from Gly104 backbone carbonyl of the antibody. |
| | Acc&Ani | Acceptor accepts an H-bond from or Anion forms a salt bridge to |
| | | guanidine side chain of receptor Arg82. This feature is marked |
| | | essential. |
| | F6 Acc2 | Directionality of F5 with respect to the side chain guanidine of |
| | | receptor Arg82. |
| | F7 Don | Derived from the side chain OH of antibody Tyr102. This OH |
| | | donates an H-bond to the backbone carbonyl of receptor Ile80. |
| | F8 Aro | Derived from side chain phenol of Tyr103 forming a favorable |
| | | hydrophobic interaction with the side chain of receptor Glu93. |
| | F9 Aro2 | Directionality of F8 with respect to the side chain of Glu93. |
| | V1 | Excluded volume |
| 6n | F1 Don | Derived from the side chain OH of antibody Tyr102. This OH |
| Partial match, | | donates an H-bond to the backbone carbonyl of receptor Ile80. |
| ligand must | F2 Don2 | Directionality of F1 with respect to the backbone carbonyl Ile80. |
| match at least 8 | F3 Acc | Derived from backbone carbonyl of antibody Tyr102 accepting |
| pharmacophore | | an H-bond from the backbone NH of receptor Glu93. |
| features. | F4 Acc2 | Directionality of F3 with respect to the backbone NH of receptor |
| | | Glu93. |
| | F5 | Derived from Gly104 backbone carbonyl of the antibody. |
| | Acc&Ani | Acceptor accepts an H-bond from or Anion forms a salt bridge to |
| | | guanidine side chain of receptor Arg82. |
| | F6 Acc2 | Directionality of F5 with respect to the side chain guanidine of |
| | | receptor Arg82. |
| | F7 Don | Derived from the side chain OH of antibody Tyr102. This OH |
| | | donates an H-bond to the backbone carbonyl of receptor Ile80. |
| | F8 Aro | Derived from side chain phenol of Tyr102, favorable coulombic |
| | | interaction with side chain guanidine of receptor Arg82. |
| | F9 Aro2 | Directionality of F8 with respect to the side chain guanidine of |
| | | receptor Arg82. |
| | V1 | Excluded volume |
| 7n | F1 Don | Derived from the backbone NH of antibody Tyr102. This NH |
| Partial match, | | donates an H-bond to the backbone carbonyl of receptor Ile91. |
| ligand must | F2 Don2 | Directionality of F1 with respect to the backbone carbonyl Ile91. |
| match at least 8 | F3 Acc | Derived from backbone carbonyl of antibody Tyr102 accepting |
| pharmacophore | | an H-bond from the backbone NH of receptor Glu93. |
| features. | F4 Acc2 | Directionality of F3 with respect to the backbone NH of receptor |
| | | Glu93. |
| | F5 | Derived from Gly104 backbone carbonyl of the antibody. |
| | Acc&Ani | Acceptor accepts an H-bond from or Anion forms a salt bridge to |
| | | guanidine side chain of receptor Arg82. |
| | F6 Acc2 | Directionality of F5 with respect to the side chain guanidine of |
| | | receptor Arg82. |
| | F7 Don | Derived from the side chain OH of antibody Tyr102. This OH |
| | | donates an H-bond to the backbone carbonyl of receptor Ile80. |
| | F8 Aro | Derived from side chain phenol of antibody Tyr102, favorable |
| | | coulombic interaction with side chain guanidine of receptor |
| | | Arg82. |
| | F9 Aro2 | Directionality of F8 with respect to the side chain guanidine of |
| | | receptor Arg82. |
| | F10 Don | Derived from the side chain OH of antibody Tyr101. This OH |
| | | donates an H-bond to the side chain carboxylate of receptor |
| | | Glu93. |
| | F11 | Directionality of F10 with respect to the side chain carboxylate of |
| | Don2 | receptor Glu93. |
| | V1 | Excluded volume |
| 10b | F1 | Derived from side chain OH of antibody Tyr102 donating an H- |
| Partial match, | Don&Acc | bond to the backbone carbonyl of receptor Ile80. |
| ligand must | F2 Acc | Derived from Gly104 backbone carbonyl of the antibody which |
| match at least 5 | | accepts an H-bond from guanidine side chain of receptor Arg82. |
| pharmacophore | F3 | Derived from the side chain OH of antibody Ser106 donating an |
| features. All | Don&Acc | H-bond to an imidazole ring nitrogen of His90. |
| pharmacophore | F4 Don | Derived from the backbone NH of antibody Tyr102. This NH |
| feature spheres | | donates an H-bond to the backbone carbonyl of receptor Ile91. |
| have a 0.8 | F5 Acc | Derived from backbone carbonyl of antibody Tyr102 accepting |
| Angstrom | | an H-bond from the backbone NH of receptor Glu93. |
| radius. | F6 Aro | Derived from side chain phenol of Tyr102, favorable coulombic |
| | | interaction with side chain guanidine of receptor Arg82. |

HER2 Complexed with Antibody (1N8Z.pdb)

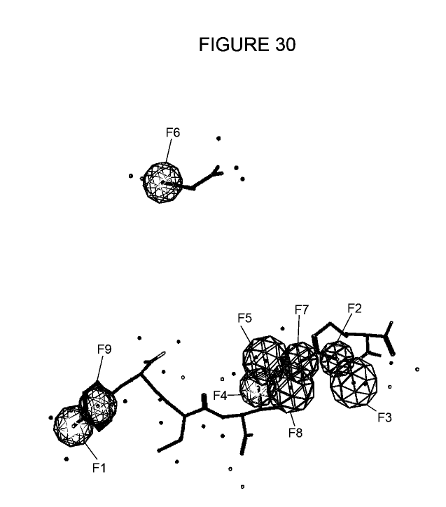

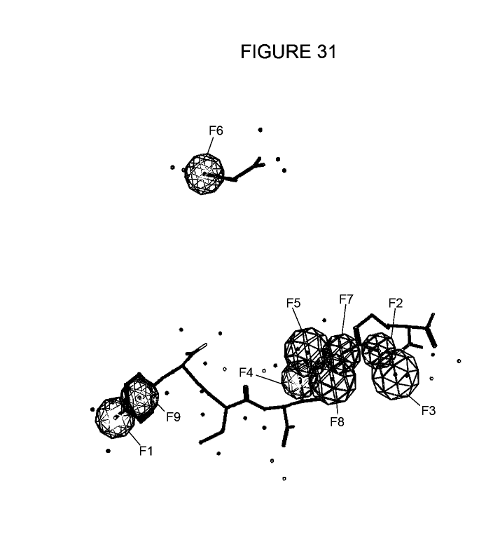

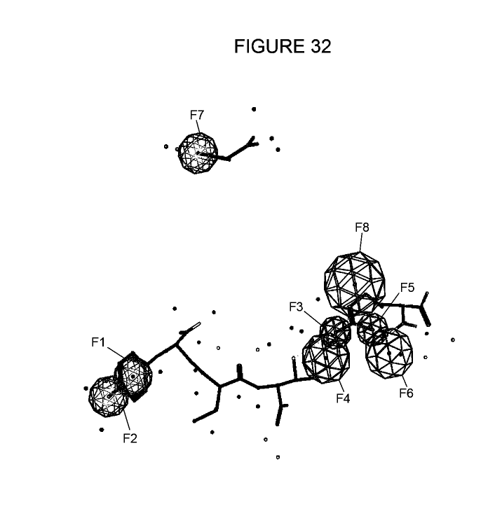

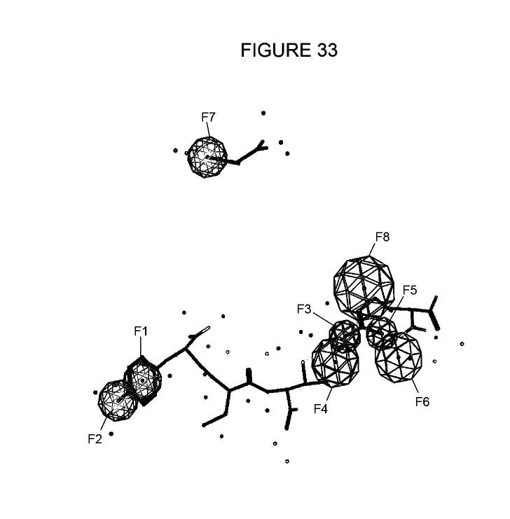

The crystal (1N8Z.pdb) of protein HER2 (SEQ ID NO: 3) complexed with antibody trastuzmab (SEQ ID NO: 7 and SEQ ID NO: 8) was analyzed according to the procedures described above. Results showed five residues of the antibody make contact with the receptor. These are Arg50, Tyr92-Thr94, and Gly103. These residues of the antibody are in close proximity to each other. They were used to generate one group of pharmacophore models described below in Table 19 and FIGS. 30-33.

TABLE 19
Pharmacophores of 1N8Z.pdb crystal of protein HER2 complexed with antibody trastuzumab

| | | |
|---|---|---|
| 1b | F1 | Derived from the side chain OH of the antibody Tyr92 accepting |
| Partial match, | Don&Acc | an H-bond from the side chain NH3+ of receptor Lys569. |
| ligand must | F2 | Derived from the side chain guanidine of antibody Arg50 |
| match at least 5 | Don&Cat | donating an H-bond to the side chain carboxylate of receptor |
| pharmacophore | | Glu558. |
| features. | F3 Don2 | Directionality of F2 with respect to the side chain carboxylate of |
| | | receptor Glu558 |
| | F4 Acc | Derived from the side chain of antibody Thr94. |
| | F5 Don2 | Directionality of F7. |
| | F6 Acc | Derived from the backbone carbonyl of antibody Gly103 |
| | | accepting an H-bond from the side chain NH3+ of Lys593. |
| | F7 | Derived from the side chain guanidine of antibody Arg50 |
| | Don&Cat | donating an H-bond to the side chain carboxylate of receptor |
| | | Asp560. |
| | F8 Don2 | Directionality of F7 with respect to the side chain carboxylate of |
| | | receptor Asp560. |
| | F9 Aro | Derived from side chain antibody Lys569 forming a favorable |
| | | hydrophobic interaction with the side pyrrolidine ring of Pro571. |
| | V1 | Excluded volume |
| 2b | | Pharmacophore model 2b is the same as 1b with the following |
| Partial match, | | exceptions: |
| ligand must | | F2, F3, F4, F5, F7 and F8 are marked essential. |
| match at least 6 | | |
| pharmacophore | | |
| features. | | |
| 2n | F1 Aro | Derived from side chain antibody Lys569 forming a favorable |
| Partial match, | | hydrophobic interaction with the side pyrrolidine ring of Pro571. |
| ligand must | | This feature is marked essential. |
| match at least 5 | F2 Acc | Derived from the side chain OH of antibody Tyr92 accepting an |
| pharmacophore | | H-bond from the side chain NH3+ of receptor Lys569. |
| features. | F3 | Derived from the side chain guanidine of antibody Arg50 |
| | Don&Cat | donating an H-bond to the side chain carboxylate of receptor |
| | | Asp560. This feature is marked essential. |
| | F4 Don2 | Directionality of F3 with respect to the side chain carboxylate of |
| | | receptor Asp560. |
| | F5 | Derived from the side chain guanidine of antibody Arg50 |
| | Don&Cat | donating an H-bond to the side chain carboxylate of receptor |
| | | Glu558. |
| | F6 Don2 | Directionality of F5 with respect to the side chain carboxylate of |
| | | receptor Glu558. |
| | F7 Acc | Derived from the backbone carbonyl of antibody Gly103 |
| | | accepting an H-bond from the side chain NH3+ of Lys593. |
| | F8 Hyd | hydrophobe, sphere radius 1.8 Angstroms, colored dark green): |
| | | Derived from MFSS. Hydrophobe forms a favorable hydrophobic |
| | | interaction with the side chain phenyl of receptor Phe573. |
| | V1 | Excluded volume |
| 3n | F1 Aro | Derived from side chain antibody Lys569 forming a favorable |
| Partial match, | | hydrophobic interaction with the side pyrrolidine ring of Pro571. |
| ligand must | F2 Acc | Derived from the side chain OH of antibody Tyr92 accepting an |
| match at least 3 | | H-bond from the side chain NH3+ of receptor Lys569. |
| pharmacophore | F3 | Derived from the side chain guanidine of antibody Arg50 |
| features. | Don&Cat | donating an H-bond to the side chain carboxylate of receptor |
| | | Asp560. This feature is marked essential. The sphere radius is |
| | | 1.0 Angstroms. |
| | F4 Don2 | Directionality of F3 with respect to the side chain carboxylate of |
| | | receptor Asp560. |
| | F5 | Derived from the side chain guanidine of antibody Arg50 |
| | Don&Cat | donating an H-bond to the side chain carboxylate of receptor |
| | | Glu558. |
| | F6 Don2 | Directionality of F5 with respect to the side chain carboxylate of |
| | | receptor Glu558. |
| | F7 Acc | Derived from the backbone carbonyl of antibody Gly103 |
| | | accepting an H-bond from the side chain NH3+ of Lys593. |
| | F8 Hyd | (hydrophobe, sphere radius 1.8 Angstroms, colored dark green): |
| | | Derived from MFSS. Forms a favorable hydrophobic interaction |
| | | with the side chain phenyl of receptor Phe573. |
| | V1 | Excluded volume |

ErbB2 Complexed with Antibody (1S78.pdb)

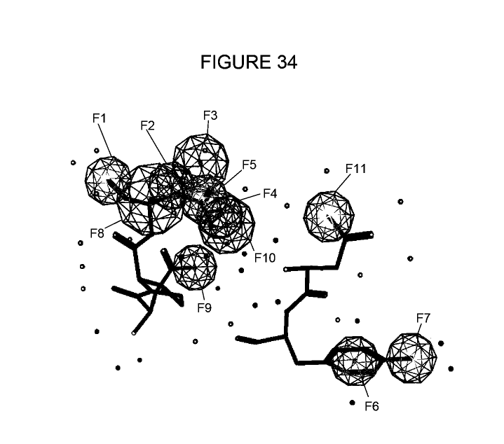

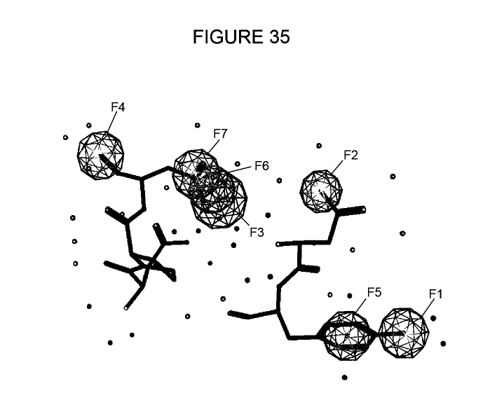

The crystal (1S78.pdb) of protein ERBB2 (SEQ ID NO: 4) complexed with antibody pertuzumab (SEQ ID NO: 9 and SEQ ID NO: 10) was analyzed according to the procedures described above. Results showed five residues of the antibody make contact with the receptor. These are Asp31-Tyr32 and Asn52-Pro52A-Asn53. These residues of the antibody are in close proximity to each other. They were used to generate the two pharmacophore models described below in Table 20 and FIGS. 34-35.

TABLE 20
Pharmacophores of 1S78.pdb crystal of protein ErbB2 complexed with antibody pertuzumab

| | | |
|---|---|---|
| 5n | F1 Acc | Derived from the backbone carbonyl of antibody Asn53 |
| Partial match, | | accepting an H-bond from the backbone NH of Cys246. |
| ligand must | F2 Don | Derived from hydrophilic contact statistics donating an H-bond to |
| match at least 8 | | the backbone carbonyl of receptor Gly287. |
| pharmacophore | F3 Don2 | Directionality of F2 with respect to the backbone carbonyl of |
| features. | | receptor Gly287. |
| | F4 Don | Derived from the side chain NH2 of antibody Asn53 donating an |
| | | H-bond to the backbone carbonyl of receptor Val286. |
| | F5 Acc | Derived from the side chain carbonyl of antibody Asn53 |
| | | accepting an H-bond from the side chain OH of receptor Thr268. |
| | F6 Aro | Derived from the side chain phenol ring of antibody Tyr32 |
| | | forming a favorable hydrophobic interaction with the pyrrolidine |
| | | ring of receptor Pro294. |
| | F7 Acc | Derived from the side chain OH of antibody Tyr32 accepting an |
| | | H-bond from the backbone carbonyl of receptor Leu295. |
| | F8 Hyd | (1.8 Angstrom sphere): Derived from MFSS forming a favorable |
| | | hydrophobic interaction with the side chain of receptor Cys246. |
| | F9 Don | Derived from the side chain NH2 of antibody Asn52 donating an |
| | | H-bond to the backbone carbonyl of receptor Val286. |
| | F10 | Directionality of F9 with respect to the backbone carbonyl of |
| | Don2 | receptor Val286. |
| | F11 | Derived from the side chain carboxylate of antibody Asp31 |
| | Acc&Ani | accepting an H-bond from the side chain OH of receptor Ser288. |
| | V1 | Excluded volume |
| 6b | F1 Acc | Derived from the side chain OH of antibody Tyr32 accepting an |
| Partial match, | | H-bond from the backbone carbonyl of receptor Leu295. |
| ligand must | F2 | Derived from the side chain carboxylate of antibody Asp31 |
| match at least 5 | Acc&Ani | accepting an H-bond from the side chain OH of receptor Ser288. |
| pharmacophore | | This feature is marked essential. |
| features. | F3 Don2 | Directionality of F6. |
| | F4 Acc | Derived from the backbone carbonyl of antibody Asn53 |
| | | accepting an H-bond from the backbone NH of Cys246. This |
| | | feature is marked essential. |
| | F5 Aro | Derived from the side chain phenol ring of antibody Tyr32 |
| | | forming a favorable hydrophobic interaction with the pyrrolidine |
| | | ring of receptor Pro294. |
| | F6 Don | Derived from the side chain NH2 of antibody Asn53 donating an |
| | | H-bond to the backbone carbonyl of receptor Val286. |
| | F7 Acc | Derived from the side chain carbonyl of antibody Asn53 |
| | | accepting an H-bond from the side chain OH of receptor Thr268. |
| | | This feature is marked essential. |
| | V1 | Excluded volume |

EGFR Complexed with the Heavy Chain of Antibody Cetuximab (2EXQ.pdb)

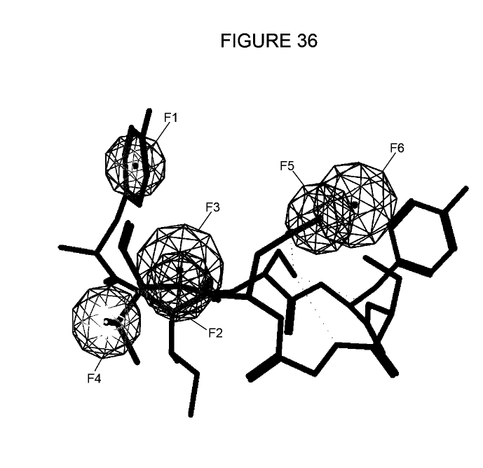

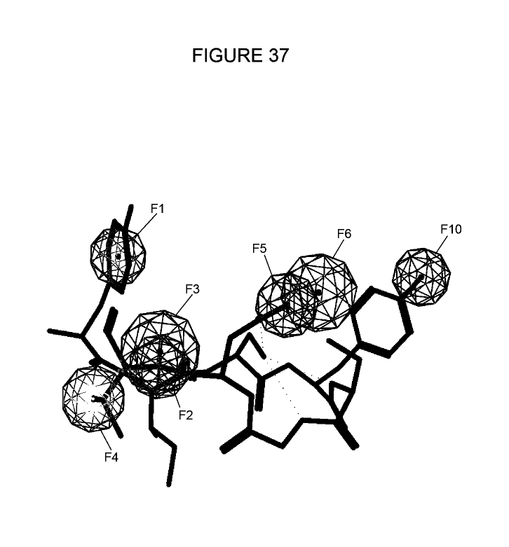

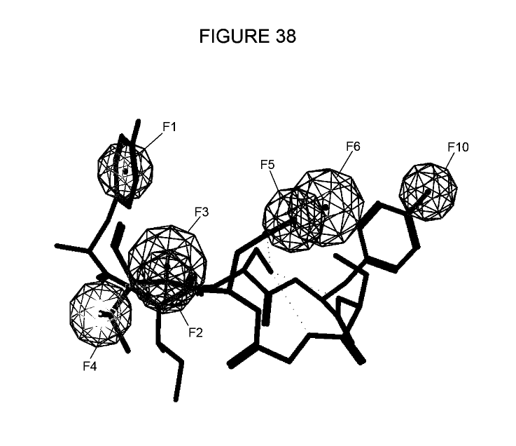

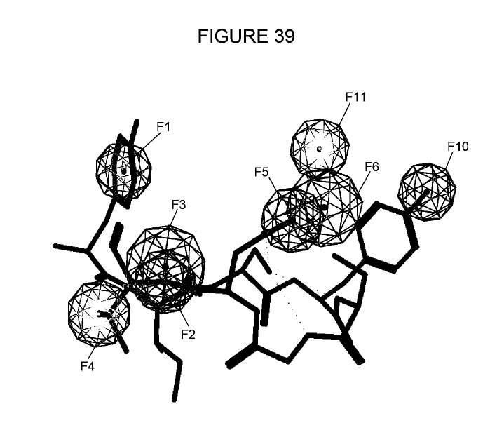

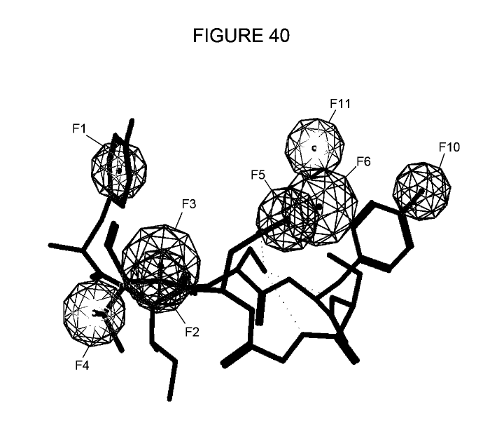

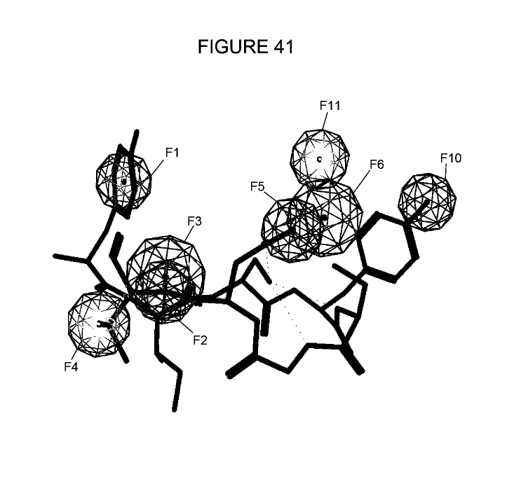

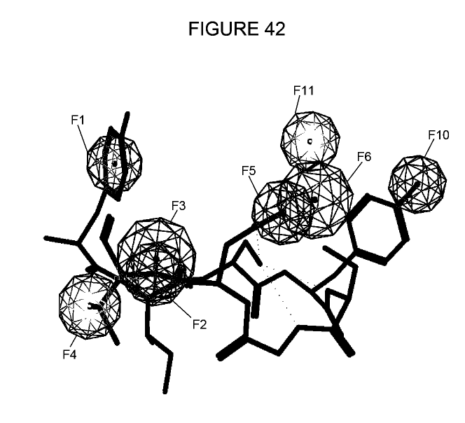

The crystal (2EXQ.pdb) of protein EGFR (SEQ ID NO: 1) complexed with the heavy chain of antibody Cetuximab (SEQ ID NO: 5 and SEQ ID NO:6) was analyzed according to the procedures described above. Results showed that, in the first set of pharmacophore models, eight residues of the heavy chain of the antibody make contact with the receptor. These are Tyr50-Thr57. They were used to generate the seven pharmacophore models described below in Table 21 and FIGS. 36-42.

TABLE 21
Pharmacophores of 2EXQ.pdb crystal of protein EGFr complexed
with heavy chain of antibody cetuximab

| | | |
|---|---|---|
| 3h | F1 Aro | Derived from the side chain phenol of antibody Tyr50 forming a |
| Partial match, | | favorable hydrophobic interaction with the side chain of receptor |
| ligand must | | Lys 303. This feature is marked essential. |
| match at least 5 | F2 Don | Derived from the backbone NH of antibody Thr57 donating an H- |
| pharmacophore | | bond to the backbone carbonyl of receptor Lys304. |
| features. | F3 Don2 | Directionality of F2 with respect to the backbone carbonyl of |
| | | receptor Lys304. |
| | F4 | Derived from the side chain OH of antibody Thr57 accepting an |
| | Acc&Ani | H-bond from the side chain NH3+ of Lys304. This feature is |
| | | marked essential. |
| | F5 Don | Derived from the side chain NH2 of antibody Asn56 donating an |
| | | H-bond to the side chain carboxylate of receptor Glu293. |
| | F6 Don2 | Directionality of F5 with respect to the side chain carboxylate of |
| | | receptor Glu293. |
| | V1 | Excluded volume |
| 4h | F1 Aro | Derived from the side chain phenol of antibody Tyr50 forming a |
| Partial match, | | favorable hydrophobic interaction with the side chain of receptor |
| ligand must | | Lys 303. |
| match at least 5 | F2 Don | Derived from the backbone NH of antibody Thr57 donating an H- |
| pharmacophore | | bond to the backbone carbonyl of receptor Lys304. |
| features. | F3 Don2 | Directionality of F2 with respect to the backbone carbonyl of |
| | | receptor Lys304. |
| | F4 | Derived from the side chain OH of antibody Thr57 accepting an |
| | Acc&Ani | H-bond from the side chain NH3+ of Lys304. This feature is |
| | | marked essential. |
| | F5 Don | Derived from the side chain NH2 of antibody Asn56 donating an |
| | | H-bond to the side chain carboxylate of receptor Glu293. |
| | F6 Don2 | Directionality of F5 with respect to the side chain carboxylate of |
| | | receptor Glu293. |
| | F7-F9 | ignored |
| | F10 Don | Derived from the side chain OH of antibody Tyr 53 donating an |
| | | H-bond to the backbone carbonyl of receptor Tyr 292. This |
| | | feature is marked essential. |
| | V1 | Excluded volume |
| 5h | F1 Aro | Derived from the side chain phenol of antibody Tyr50 forming a |
| Partial match, | | favorable hydrophobic interaction with the side chain of receptor |
| ligand must | | Lys 303. This feature is marked essential. |
| match at least 5 | F2 Don | Derived from the backbone NH of antibody Thr57 donating an H- |
| pharmacophore | | bond to the backbone carbonyl of receptor Lys304. |
| features. | F3 Don2 | Directionality of F2 with respect to the backbone carbonyl of |
| | | receptor Lys304. |
| | F4 | Derived from the side chain OH of antibody Thr57 accepting an |
| | Acc&Ani | H-bond from the side chain NH3+ of Lys304. This feature is |
| | | marked essential. |
| | F5 Don | Derived from the side chain NH2 of antibody Asn56 donating an |
| | | H-bond to the side chain carboxylate of receptor Glu293. |
| | F6 Don2 | Directionality of F5 with respect to the side chain carboxylate of |
| | | receptor Glu293. |
| | F7-F9 | ignored |
| | F10 Don | Derived from the side chain OH of antibody Tyr 53 donating an |
| | | H-bond to the backbone carbonyl of receptor Tyr 292. |
| | V1 | Excluded volume |
| 6h | F1 Aro | Derived from the side chain phenol of antibody Tyr50 forming a |
| Partial match, | | favorable hydrophobic interaction with the side chain of receptor |
| ligand must | | Lys 303. This feature is marked essential. |
| match at least 4 | F2 Don | Derived from the backbone NH of antibody Thr57 donating an H- |
| pharmacophore | | bond to the backbone carbonyl of receptor Lys304. |
| features. | F3 Don2 | Directionality of F2 with respect to the backbone carbonyl of |
| | | receptor Lys304. |
| | F4 | Derived from the side chain OH of antibody Thr57 accepting an |
| | Acc&Ani | H-bond from the side chain NH3+ of Lys304. |
| | F5 Don | Derived from the side chain NH2 of antibody Asn56 donating an |
| | | H-bond to the side chain carboxylate of receptor Glu293. |
| | F6 Don2 | Directionality of F5 with respect to the side chain carboxylate of |
| | | receptor Glu293. |
| | F7-F9 | ignored |
| | F10 Don | Derived from the side chain OH of antibody Tyr 53 donating an |
| | | H-bond to the backbone carbonyl of receptor Tyr 292. |
| | F11 | Derived from hydrophilic contact statistics. This feature accepts |
| | Acc&Ani | an H-bond from the backbone NH of receptor Met294 and/or the |
| | | side chain NH₃⁺ of receptor Lys303 and/or forms a salt bridge |
| | | with the side chain NH₃⁺ of receptor Lys303. This feature is |
| | | marked essential. |
| | V1 | Excluded volume |
| 7h | F1 Aro | Derived from the side chain phenol of antibody Tyr50 forming a |
| Partial match, | | favorable hydrophobic interaction with the side chain of receptor |
| ligand must | | Lys 303. |
| match at least 4 | F2 Don | Derived from the backbone NH of antibody Thr57 donating an H- |
| pharmacophore | | bond to the backbone carbonyl of receptor Lys304. |
| features. | F3 Don2 | Directionality of F2 with respect to the backbone carbonyl of |
| | | receptor Lys304. |
| | F4 | Derived from the side chain OH of antibody Thr57 accepting an |
| | Acc&Ani | H-bond from the side chain NH3+ of Lys304. This feature is |
| | | marked essential. |
| | F5 Don | Derived from the side chain NH2 of antibody Asn56 donating an |
| | | H-bond to the side chain carboxylate of receptor Glu293. |
| | F6 Don2 | Directionality of F5 with respect to the side chain carboxylate of |
| | | receptor Glu293. |
| | F7-F9 | ignored |
| | F10 Don | Derived from the side chain OH of antibody Tyr 53 donating an |
| | | H-bond to the backbone carbonyl of receptor Tyr 292. |
| | F11 | Derived from hydrophilic contact statistics. This feature accepts |
| | Acc&Ani | an H-bond from the backbone NH of receptor Met294 and/or the |
| | | side chain NH₃⁺ of receptor Lys303 and/or forms a salt bridge |
| | | with the side chain NH₃⁺ of receptor Lys303. This feature is |
| | | marked essential. |
| | V1 | Excluded volume |
| 8h | F1 Aro | Derived from the side chain phenol of antibody Tyr50 forming a |
| Partial match, | | favorable hydrophobic interaction with the side chain of receptor |
| ligand must | | Lys 303. |
| match at least 4 | F2 Don | Derived from the backbone NH of antibody Thr57 donating an H- |
| pharmacophore | | bond to the backbone carbonyl of receptor Lys304. |
| features. | F3 Don2 | Directionality of F2 with respect to the backbone carbonyl of |
| | | receptor Lys304. |
| | F4 | Derived from the side chain OH of antibody Thr57 accepting an |
| | Acc&Ani | H-bond from the side chain NH3+ of Lys304. |
| | F5 Don | Derived from the side chain NH2 of antibody Asn56 donating an |
| | | H-bond to the side chain carboxylate of receptor Glu293. This |
| | | feature is marked essential. |
| | F6 Don2 | Directionality of F5 with respect to the side chain carboxylate of |
| | | receptor Glu293. |
| | F7-F9 | ignored |
| | F10 Don | Derived from the side chain OH of antibody Tyr 53 donating an |
| | | H-bond to the backbone carbonyl of receptor Tyr 292. |
| | F11 | Derived from hydrophilic contact statistics. This feature accepts |
| | Acc&Ani | an H-bond from the backbone NH of receptor Met294 and/or the |
| | | side chain NH₃⁺ of receptor Lys303 and/or forms a salt bridge |
| | | with the side chain NH₃⁺ of receptor Lys303. This feature is |
| | | marked essential. |
| | V1 | Excluded volume |
| 9h | F1 Aro | Derived from the side chain phenol of antibody Tyr50 forming a |
| Partial match, | | favorable hydrophobic interaction with the side chain of receptor |
| ligand must | | Lys 303. |
| match at least 4 | F2 Don | Derived from the backbone NH of antibody Thr57 donating an H- |
| pharmacophore | | bond to the backbone carbonyl of receptor Lys304. This feature |
| features. | | is marked essential. |
| | F3 Don2 | Directionality of F2 with respect to the backbone carbonyl of |
| | | receptor Lys304. |
| | F4 | Derived from the side chain OH of antibody Thr57 accepting an |
| | Acc&Ani | H-bond from the side chain NH3+ of Lys304. |
| | F5 Don | Derived from the side chain NH2 of antibody Asn56 donating an |
| | | H-bond to the side chain carboxylate of receptor Glu293. This |
| | | feature is marked essential. |
| | F6 Don2 | Directionality of F5 with respect to the side chain carboxylate of |
| | | receptor Glu293. |
| | F7-F9 | ignored |
| | F10 Don | Derived from the side chain OH of antibody Tyr 53 donating an |
| | | H-bond to the backbone carbonyl of receptor Tyr 292. |
| | F11 | Derived from hydrophilic contact statistics. This feature accepts |
| | Acc&Ani | an H-bond from the backbone NH of receptor Met294 and/or the |
| | | side chain NH₃⁺ of receptor Lys303 and/or forms a salt bridge |
| | | with the side chain NH₃⁺ of receptor Lys303. This feature is |
| | | marked essential. |
| | V1 | Excluded volume |

EGFr Complexed with the Light Chain of Antibody Cetuximab (2EXQ.pdb)

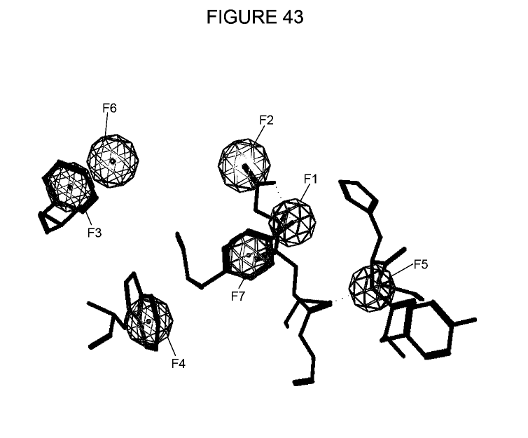

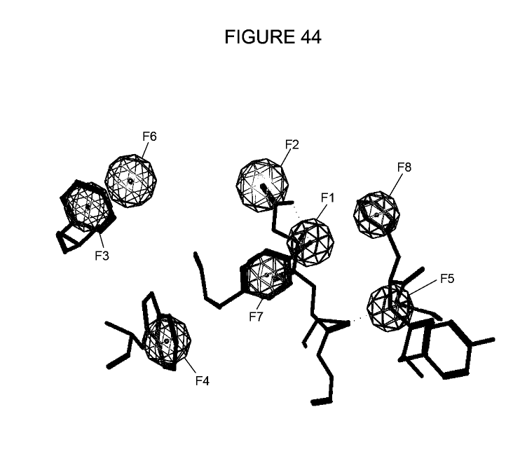

The crystal (2EXQ.pdb) of protein EGFR (SEQ ID NO: 1) complexed with the light chain of antibody Cetuximab (SEQ ID NO: 5 and SEQ ID NO:6) was analyzed according to the procedures described above. Results showed that, in the first set of pharmacophore models, nine residues of the light chain of the antibody make contact with the receptor. These are Asn32_Ile33_Gly34, Tyr49_His50_Gly51, Tyr91, Phe94, and Trp96. They were used to generate the six pharmacophore models described below in Table 22 and FIGS. 43-44.

TABLE 22
Pharmacophores of 2EXQ.pdb crystal of protein EGFr complexed
with light chain of antibody cetuximab

| | | |
|---|---|---|
| 1L | F1 Don | Derived from the side chain OH of antibody Tyr91 donating an |
| Partial match, | | H-bond to the backbone carbonyl of receptor Asp297. |
| ligand must | F2 | Derived from the side chain carbonyl of antibody Asn32 |
| match at least 4 | Acc&Ani | accepting an H-bond from or forming a salt bridge with the side |
| pharmacophore | | chain NH₃⁺ of receptor Lys 301. |
| features. | F3 Aro | Derived from the side chain phenyl of antibody Phe94 forming a |
| | | favorable coulombic interaction with the side chain NH3+ of |
| | | receptor Lys304. |
| | F4 Aro | Derived from the side chain phenyl of antibody Trp96 forming a |
| | | favorable coulombic interaction with the side chain carboxylate |
| | | of receptor Glu296. |
| | F5 Don | Derived from the backbone NH of antibody His50 donating an H- |
| | | bond to the side chain carboxylate of receptor Asp297. |
| | F6 | Derived from hydrophilic contact statistics. This feature accepts |
| | Acc&Ani | an H-bond from or forms a salt bridge with the side chain NH₃⁺ |
| | | of receptor Lys 303. This feature is marked essential. |
| | F7 Aro | Derived from the side chain phenol of antibody Tyr91 forming a |
| | | favorable hydrophobic interaction with the side chain of receptor |
| | | Asp297. |
| | V1 | Excluded volume |
| 2L | | This model is the same as 1L except that both F2 Acc&Ani and |
| Partial match, | | F6 Acc&Ani are marked essential. |
| ligand must | | |
| match at least 3 | | |
| pharmacophore | | |
| features. | | |
| 3L | F1 Don | Derived from the side chain OH of antibody Tyr91 donating an |
| Partial match, | | H-bond to the backbone carbonyl of receptor Asp297. |
| ligand must | F2 | Derived from the side chain carbonyl of antibody Asn32 |
| match at least 4 | Acc&Ani | accepting an H-bond from or forming a salt bridge with the side |
| pharmacophore | | chain NH₃⁺ of receptor Lys 301. |
| features. | F3 Aro | Derived from the side chain phenyl of antibody Phe94 forming a |
| | | favorable coulombic interaction with the side chain NH3+ of |
| | | receptor Lys304. |
| | F4 Aro | Derived from the side chain phenyl of antibody Trp96 forming a |
| | | favorable coulombic interaction with the side chain carboxylate |
| | | of receptor Glu296. |
| | F5 Don | Derived from the backbone NH of antibody His50 donating an H- |
| | | bond to the side chain carboxylate of receptor Asp297. |
| | F6 | Derived from hydrophilic contact statistics. This feature accepts |
| | Acc&Ani | an H-bond from or forms a salt bridge with the side chain NH₃⁺ |
| | | of receptor Lys 303. This feature is marked essential. |
| | F7 Aro | Derived from the side chain phenol of antibody Tyr91 forming a |
| | | favorable hydrophobic interaction with the side chain of receptor |
| | | Asp297. This feature is marked essential. |
| | F8 Aro | Derived from the imidazole side chain of antibody His50 forming |
| | | a favorable coulombic interaction with the carboxylate side chain |
| | | of receptor Asp297. |
| | V1 | Excluded volume |
| 5L | | This model is the same as 3L except that the F1 Don is marked |
| Partial match, | | essential. |
| ligand must | | |
| match at least 4 | | |
| pharmacophore | | |
| features. | | |
| 6L | | This model is the same as 3L except that the F1 Don, the F2 |
| Partial match, | | Acc&Ani and the F3 Aro are marked essential. |
| ligand must | | |
| match at least 3 | | |
| pharmacophore | | |
| features. | | |
| 7L | | This model is the same as 3L except that the F1 Don, the F2 |
| Partial match, | | Acc&Ani and the F5 Don are marked essential. |
| ligand must | | |
| match at least 4 | | |
| pharmacophore | | |
| features. | | |

Using the above described methodology, one can generate pharmacophore models for a variety of protein targets (crystallized with ligand) including, but not limited to: Foot and Mouth Disease (1QGC.pdb); Angiotensin II (1CK0.pdb, 3CK0.pdb, 2CK0.pdb); ErbB2 complexed with pertuzumab antibody (1 L71.pdb, 1S78.pdb, 2GJJ.pdb); Flu Agglutinin (1DN0.pdb, 1OSP.pdb); Flu Hemagglutinin (1EO8.pdb, 1QFU.pdb, 2VIR.pdb, 2VIS.pdb, 2VIT.pdb, 1KEN.pdb, 1FRG.pdb, 1HIM.pdb, 1HIN.pdb, 11FH.pdb); Flu Neuraminidase (NC10.pdb, 1A14.pdb, 1NMB.pdb, 1NMC.pdb, 1NMA.pdb, 1NCA.pdb, 1NCD.pdb, 2AEQ.pdb, 1NCB.pdb, 1NCC.pdb, 2AEP.pdb); Gamma Interferon (HuZAF.pdb, 1T3F.pdb, 1B2W.pdb, 1B4J.pdb, 1T04.pdb); HER2 complexed with Herceptin (1N8Z.pdb, 1FVC.pdb); Neisseria Meningitidis (1 MNU.pdb, 1 MPA.pdb, 2 MPA.pdb, 1UWX.pdb); HIV1 Protease (1JP5.pdb, 1CL7.pdb, 1MF2.pdb, 2HRP.pdb, 1SVZ.pdb); HIV-1 Reverse Transcriptase (2HMI.pdb, 1J50.pdb, 1N5Y.pdb, 1N6Q.pdb, 1HYS.pdb, 1C9R.pdb, 1HYS.pdb, 1R08.pdb, 1T04.pdb, 2HRP.pdb); Rhinovirus (1FOR.pdb, 1RVF.pdb, 1BBD.pdb, 1A3R.pdb, 1A6T.pdb); platelet fibrinogen receptor (1TXV.pdb, 1TY3.pdb, 1TY5.pdb, 1TY6.pdb, 1TY7.pdb); Salmonella oligosaccharide (1 MFB.pdb, 1MFC.pdb, 1 MFE.pdb); TGF-Alpha (1E4W.pdb, 1E4X.pdb); Thrombopoietin complexed with TN1 (1V7M.pdb, 1V7N.pdb); Tissue Factor complexed with 5G9 (1FGN.pdb, 1AHW.pdb, 1JPS.pdb, 1UJ3.pdb); Von Willenbrand Factor complexed with NMC-4 (1OAK.pdb, 2ADF.pdb, 1FE8.pdb, 1FNS.pdb, 2ADF.pdb); VEGF complexed with B20-4 (2FJH.pdb, 2FJF.pdb, 2FJG.pdb, 1TZH.pdb, 1TZI.pdb, 1CZ8.pdb, 1BJ1.pdb); Coronavirus—SARS (2DD8.pdb, 2G75.pdb); Lyme Disease (1P4P.pdb, 1RJL.pdb); HIV GP120 (1ACY.pdb, 1F58.pdb, 1G9M.pdb, 1G9N.pdb, 1GC1.pdb, 1Q1J.pdb, 1QNZ.pdb, 1RZ7.pdb, 1RZ8.pdb, 1RZF.pdb, 1RZG.pdb, 1RZI, 1RZJ.pdb, 1RZK.pdb, 1YYL.pdb, 1YYM.pdb, 2B4C.pdb, 2F58.pdb, 2F5A.pdb); HIV GP41 (1TJG.pdb, 1TJH.pdb, 1TJI.pdb, 1U92.pdb, 1U93.pdb, 1U95.pdb, 1U8H.pdb, 1U81.pdb, 1U8J.pdb, 1U8K.pdb, 1U8P.pdb, 1U8Q.pdb, 1U91.pdb, 1U8L.pdb, 1U8M.pdb, 1U8N.pdb, 1U80.pdb, 2F5B); West Nile Virus (as defined in US Patent App. Pub. No. 2006/0115837); Malaria (Dihydrofolate reductase) (as defined in Acta Crystallographia (2004), D60(11), 2054-2057); and EGFR (1181.pdb, 118K.pdb, 1YY8.pdb, 1YY9.pdb, 2EXP.pdb, 2EXQ.pdb).

### Example 5

### Ligand Docking and Scoring

The compounds selected for docking to the target protein were those which were found to align to the pharmacophore models generated in the MOE modeling software (see Example 4). These compounds were obtained in MOE database format from the ZINC database (see Irwin and Shoichet (2005) J Chem Inf Model 45, 177-182). The 3-dimensional atomic coordinates of these compounds were written to a structure data format (*.sdf) file using the export command in the MOE database window without adding hydrogens.

The LigPrep software module of Maestro modeling software (Schrodinger LLC, NY, N.Y.) was next employed to prepare the compounds for docking. The *.sdf file was converted into Maestro format using LigPrep. Hydrogens were then added and any charged groups neutralized. Ionization states were generated for the ligands at 7.0+/−1.0 pH units. After this, tautomers were generated when necessary, alternate chiralities were generated and low energy ring conformers were produced. This was followed by removing any problematic structures and energy minimizing the resulting ligands using MacroModel software module. Finally a Maestro file (*.mae) was written of the ligands which were now ready for docking. All of these steps were automated via a python script supplied by Schrodinger, LLC.

The following describes protein preparation. First a protein was imported into Maestro in PDB format. Hydrogens were added and any errors such as incomplete residues were repaired. The protein structure was checked for metal ions and cofactors. Charges and atom types were set for metal ions and cofactors as needed. Ligand bond orders and formal charges were adjusted if necessary. The binding site was determined by picking the ligand (for 1YY9 it is either the Thr100-Tyr101-Tyr102-Asp103-Tyr104-Glu105 or Gly54-Gly55-Asn56-Thr57-Asp58 pieces of the antibody) in Maestro (Glide). The program determines the centroid of the picked ligand and draws a 20 Angstrom box which represents the default setting with the centroid of the ligand at the center of the box. The box was the binding site for the ligands to be docked. The protein preparation facility, which is automated in Glide, consists of two components, preparation and refinement. The preparation component added hydrogens and neutralized side chains that are not close to the binding site and do not participate in salt bridges. The refinement component performed a restrained minimization of the co-crystallized complex which reoriented side-chain hydroxyl groups and alleviated potential steric clashes.

The following describes receptor grid generation. Glide searches for favorable interactions between one or more ligand molecules and a receptor molecule, usually a protein. The shape and properties of the receptor are represented on a grid by several different sets of fields including hydrogen bonding, coulombic (i.e., charge-charge) interactions hydrophobic interactions, and steric clashes of the ligand with the protein. In the first step the receptor must be defined. This was done by picking the ligand. The unpicked part of the structure was the receptor. The ligand was not included in the grid calculation but was used to define the binding site as described above. Scaling of the nonpolar atoms of the receptor was not included in the present docking runs. The grids themselves were calculated within the space of the enclosing box. This is the box described above and all of the ligand atoms must be contained in this box. No pharmacophore constraints were used because the Glide extra precision scoring function performs better without these constraints.

To use Glide, each ligand must be a single molecule, while the receptor may include more than one molecule, e.g., a protein and a cofactor. Glide can be run in rigid or flexible docking modes; the latter automatically generates conformations for each input ligand. The combination of position and orientation of a ligand relative to the receptor, along with its conformation in flexible docking, is referred to as a ligand pose. All docking runs are done using the flexible docking mode. The ligand poses that Glide generates pass through a series of hierarchical filters that evaluate the ligand's interaction with the receptor. The initial filters test the spatial fit of the ligand to the defined active site, and examine the complementarity of ligand-receptor interactions using a grid-based method. Poses that pass these initial screens enter the final stage of the algorithm, which involves evaluation and minimization of a grid approximation to the OPLS-AA nonbonded ligand-receptor interaction energy. Final scoring is then carried out on the energy-minimized poses. By default, Schrodinger's proprietary GlideScore multi-ligand scoring function is used to score the poses. If GlideScore was selected as the scoring function, a composite Emodel score is then used to rank the poses of each ligand and to select the poses to be reported to the user. Emodel combines GlideScore, the nonbonded interaction energy, and, for flexible docking, the excess internal energy of the generated ligand conformation. Conformational flexibility is handled in Glide by an extensive conformational search, augmented by a heuristic screen that rapidly eliminates unsuitable conformations, such as conformations that have long-range internal hydrogen bonds.

The settings used in the docking runs of this example were as follows. Grid file was read in. Extra precision (XP) scoring function was used. Docked using conformational flexibility. 5000 poses per ligand for the initial Glide screen were kept (default). Scoring window for keeping initial poses was 100.0 (default). Best 800 poses per ligand for the energy minimization was kept (default). For the energy minimization, a distance dependent dielectric constant of 2.0 was used and maximum number of conjugate gradient steps was 100 (defaults). The ligand file was then loaded. Molecules with >120 atoms and/or >20 rotatable bonds were not docked (default). Van der Waals radii of ligand atoms with partial charges <0.15 were scaled by 0.80. This was done to mimic receptor flexibility. Constraints and similarity were not used. Poses with Coulomb plus Van der Waals energies >0.0 were rejected. To ensure that poses for each molecule were conformationally distinct, poses with RMS deviation <0.5 and/or maximum atomic displacement of 1.3 Angstroms were discarded.

The following describes Glide Scoring. The choice of best-docked structure for each ligand was made using a model energy score (Emodel) that combines the energy grid score, the binding affinity predicted by GlideScore, and (for flexible docking) the internal strain energy for the model potential used to direct the conformational-search algorithm. Glide also computed a specially constructed Coulomb-van der Waals interaction-energy score (CvdW) that was formulated to avoid overly rewarding charge-charge interactions at the expense of charge-dipole and dipole-dipole interactions. This score was intended to be more suitable for comparing the binding affinities of different ligands than is the “raw” Coulomb-van der Waals interaction energy. In the final data work-up, one can combine the computed GlideScore and “modified” Coulomb-van der Waals score values to give a composite score that can help improve enrichment factors in database screening applications. The mathematical form of the Glide score is:
GScore=0.065*EvdW+0.130*Coul+Lipo+Hbond+Metal+BuryP+RotB+Site

where EvdW is van der Waals energy (calculated with reduced net ionic charges on groups with formal charges, such as metals, carboxylates, and guanidiniums); Coul is the Coulomb energy (calculated with reduced net ionic charges on groups with formal charges, such as metals, carboxylates, and guanidiniums); Lipo is the lipophilic contact term (rewards favorable hydrophobic interactions); HBond is the hydrogen-bonding term (separated into differently weighted components that depend on whether the donor and acceptor are neutral, one is neutral and the other is charged, or both are charged); metal is the metal-binding term (only the interactions with anionic acceptor atoms are included; if the net metal charge in the apo protein is positive, the preference for anionic ligands is included; if the net charge is zero, the preference is suppressed); BuryP is the penalty for buried polar groups; RotB is the penalty for freezing rotatable bonds; and Site is polar interactions in the active site (polar but non-hydrogen-bonding atoms in a hydrophobic region are rewarded).

The following describes generation of the virtual compound library that was screened. The lead-like compounds from a free, virtual database of commercially available compounds was downloaded in structure data format (sdf, Molecular Design Limited) from the ZINC database (Irwin and Shoichet (2005) J. Chem. Inf. Model. 45(1), 177-182). The lead-like database is comprised of approximately 890,000 compounds divided into 33 segments. This was used to generate the database of conformers for screening by MOE. Hydrogens were then added. For a pharmacophore search, a database of low energy conformers must be generated. The Conformation Import command was applied to the sdf file above. After the conformers were generated, preprocessing of the conformer database was applied. This step, called feature annotation, determined the types of pharmacophore features in each molecule/conformation and their geometrical relationships. This was then compared with the query and those molecules/conformations that matched the query within the given tolerance were saved as hits.

EGFR

Analysis of compounds from the ZINC database against the pharmacophores identified from the 1YY9.pdb crystal of protein EGFR (SEQ ID NO: 1) complexed with antibody Cetuximab (SEQ ID NO: 5 and SEQ ID NO:6) (see e.g., Example 4; Table 17) identified 183 similar compounds. Those compounds were analyzed according to the docking and scoring methods described above. Exemplary results from the docking and scoring tests are presented in Table 23.

TABLE 23

| | | | | | Pharmacophore |
|---|---|---|---|---|---|
| ZINC# | AD4# | target | G_score | E_model | model |
| ZINC04342589 | AD4- | EGFR | −7.51718 | −36.7811 | 1_gly54_asp58 |
| | 1020 | | | | |
| ZINC00148428 | AD4- | EGFR | −7.34233 | −37.5868 | 1_gly54_asp58 |
| | 1021 | | | | |
| ZINC04649255 | AD4- | EGFR | −7.13496 | −41.4482 | 23_gly54_asp58 |
| | 1178 | | | | |
| ZINC00073705 | AD4- | EGFR | −6.9552 | −43.1905 | 23_gly54_asp58 |
| | 1142 | | | | |
| Similar to | AD4- | EGFR | −6.83 | −38.3 | Similarity - |
| ZINC04342589 | 1175 | | | | 1_gly54_asp58 |
| ZINC04824860 | AD4- | EGFR | −6.73644 | −42.2379 | 1_gly54_asp58 |
| | 1022 | | | | |
| ZINC04651153 | AD4- | EGFR | −6.69071 | −38.8887 | 1_gly54_asp58 |
| | 1070 | | | | |
| ZINC00528869 | AD4- | EGFR | −6.54409 | −45.9287 | 23_gly54_asp58 |
| | 1176 | | | | |
| ZINC04687278 | AD4- | EGFR | −6.4093 | −51.0833 | 1_gly54_asp58 |
| | 1025 | | | | |
| ZINC00459879 | AD4- | EGFR | −6.33665 | −42.7825 | 23_thr100_glu105- |
| | 1133 | | | | round 2 |
| ZINC004825941 | AD4- | EGFR | −6.28522 | −42.9355 | 23_thr100_glu105- |
| | 1132 | | | | round 2 |
| ZINC04124337 | AD4- | EGFR | −6.2615 | −31.6679 | 1_gly54_asp58 |
| | 1027 | | | | |
| ZINC01011300 | AD4- | EGFR | −6.21569 | −29.4831 | 22_thr100_glu105- |
| | 1109 | | | | round 2 |
| Similar to | AD4- | EGFR | −6.14168 | −47.3274 | Similarity to |
| ZINC05257849 | 1165 | | | | 1_thr100_glu105 |
| ZINC00062419 | AD4- | EGFR | −6.04072 | −40.5308 | 21_gly54_asp58 |
| | 1108 | | | | |
| ZINC04123287 | AD4- | EGFR | −5.90937 | −49.9531 | 23_gly54_asp58 |
| | 1128 | | | | |
| ZINC00142260 | AD4- | EGFR | −5.88248 | −32.5697 | 1_thr100_glu105 |
| | 1038 | | | | |
| ZINC00132680 | AD4- | EGFR | −5.87764 | −43.513 | 23_gly54_asp58 |
| | 1148 | | | | |
| ZINC02107327 | AD4- | EGFR | −5.78666 | −41.2423 | 1_thr100_glu105 |
| | 1047 | | | | |
| ZINC04280006 | AD4- | EGFR | −5.76102 | −47.0273 | 1_gly54_asp58 |
| | 1039 | | | | |
| ZINC00187413 | AD4- | EGFR | −5.68324 | −36.6217 | 1_gly54_asp58 |
| | 1030 | | | | |
| ZINC00060213 | AD4- | EGFR | −5.54974 | −32.537 | 10_thr100_glu105 |
| | 1057 | | | | |
| ZINC02821322 | AD4- | EGFR | −5.43675 | −32.9549 | 22_thr100_glu105 |
| | 1139 | | | | |
| ZINC04706675 | AD4- | EGFR | −5.26581 | −39.7123 | 22_thr100_glu105 |
| | 1123 | | | | |
| ZINC02998684 | AD4- | EGFR | −5.2081 | −34.3632 | 21_thr100_glu105 |
| | 1124 | | | | |
| ZINC02550733 | AD4- | EGFR | −5.1875 | −35.4196 | 1_thr100_glu105 |
| | 1009 | | | | |
| ZINC000234700 | AD4- | EGFR | −5.18527 | −36.6651 | 2_thr100_glu105 |
| | 1010 | | | | |
| ZINC02324099 | AD4- | EGFR | −5.1848 | −28.3449 | 2_thr100_glu105 |
| | 1060 | | | | |
| ZINC02972737 | AD4- | EGFR | −5.14851 | −31.5451 | 22_thr100_glu105 |
| | 1121 | | | | |
| ZINC02182988 | AD4- | EGFR | −5.09654 | −29.76 | 11_gly54_asp58 |
| | 1016 | | | | |
| ZINC00255042 | AD4- | EGFR | −5.08696 | −34.0888 | 10_thr100_glu105 |
| | 1017 | | | | |
| ZINC02666610 | AD4- | EGFR | −5.08543 | −32.1631 | 10_thr100_glu105 |
| | 1018 | | | | |
| ZINC04248154 | AD4- | EGFR | −5.01311 | −37.7306 | 22_thr100_glu105 |
| | 1141 | | | | |
| ZINC04625685 | AD4- | EGFR | −4.99755 | −49.9094 | 21_gly54_asp58 |
| | 1147 | | | | |
| Similar to | AD4- | EGFR | −4.91 | −50.2 | 1_gly54_asp58 |
| ZINC04687278 | 1167 | | | | |
| Similar to | AD4- | EGFR | −4.83 | −47.8 | 1_gly54_asp58 |
| ZINC04687278 | 1149 | | | | |
| Similar to | AD4- | EGFR | −4.74 | −49.2 | Similarity to |
| ZINC04687278 | 1171 | | | | 1_gly54_asp58 |
| Similar to | AD4- | EGFR | −4.48 | −45.6 | Similarity to |
| ZINC04687278 | 1155 | | | | 1gly54_asp58 |
| Similar to | AD4- | EGFR | −4.32091 | −39.5498 | Similarity to |
| ZINC00148428 | 1150 | | | | 1_gly54_asp58 |
| Similar to | AD4- | EGFR | −4.09 | −44.4 | Similarity to |
| ZINC04342589 | 1164 | | | | 1_gly54_asp58 |
| Similar to | AD4- | EGFR | −3.83555 | −33.6026 | Similarity to |
| ZINC04133773 | 1140 | | | | 1_thr100_glu105 |
| Similar to | AD4- | EGFR | −3.77 | −28.5 | Similarity to |
| ZINC04342589 | 1169 | | | | 1_gly54_asp58 |
| Similar to | AD4- | EGFR | −3.51745 | −50.5481 | 1_thr100_glu105 |
| ZINC05257849 | 1166 | | | | |

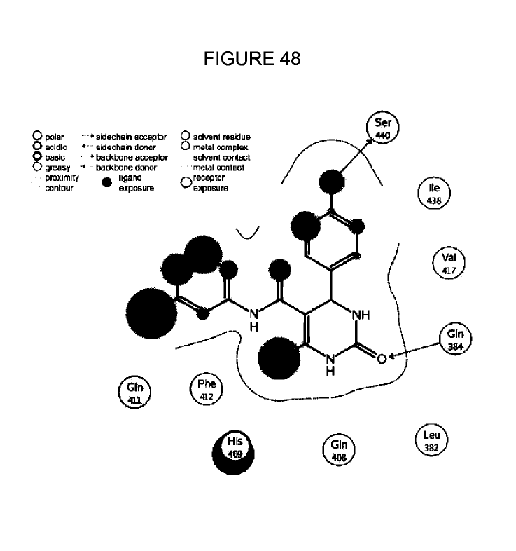

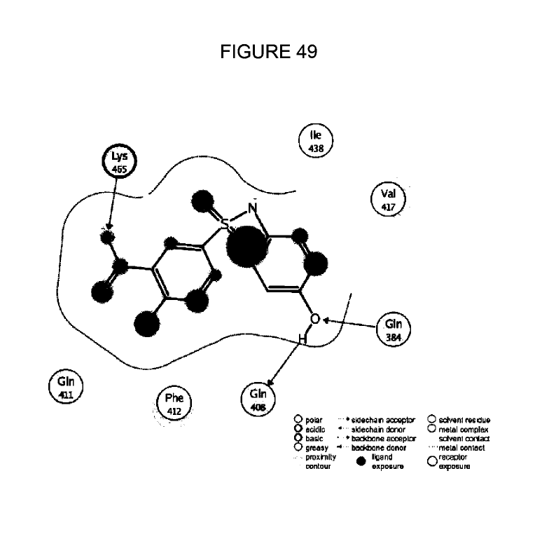

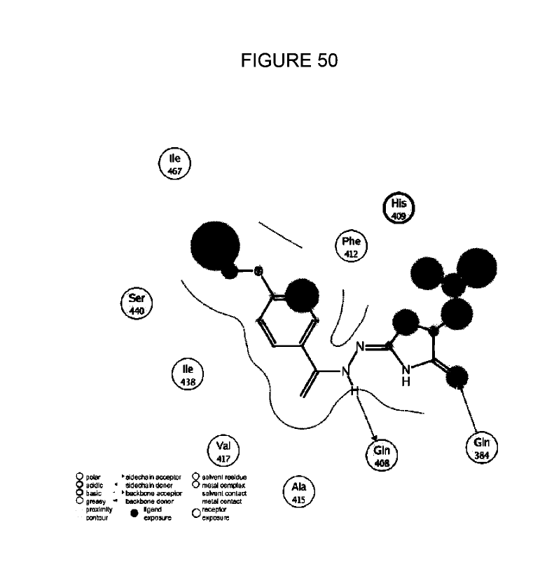

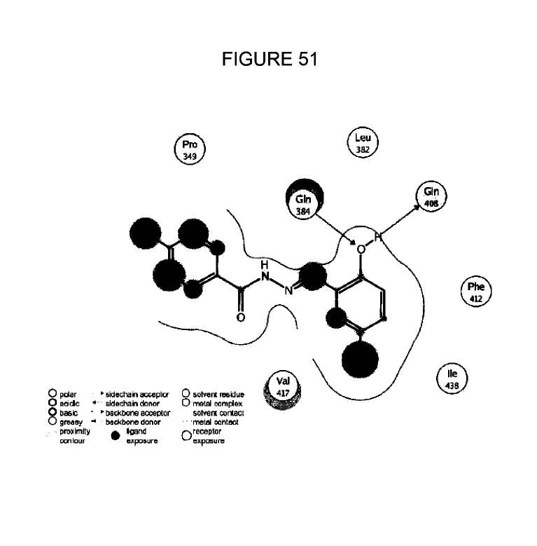

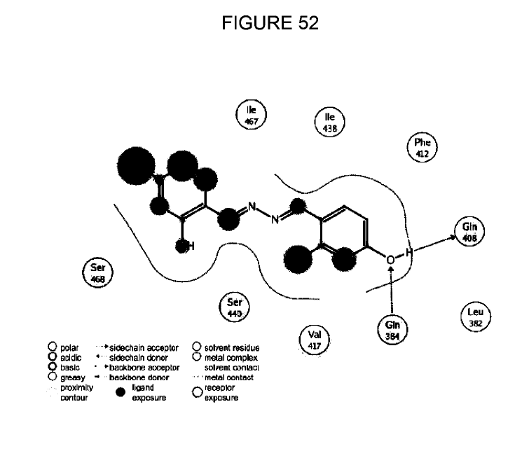

Docking of compound AD4-1009 to EGFR is depicted, for example, in FIG. 49. Docking of compound AD4-1010 to EGFR is depicted, for example, in FIG. 48. Docking of compound AD4-1016 to EGFR is depicted, for example, in FIG. 50. Docking of compound AD4-1017 to EGFR is depicted, for example, in FIG. 51. Docking of compound AD4-1018 to EGFR is depicted, for example, in FIG. 52. Docking of compound AD4-1025 to EGFR is depicted, for example, in FIG. 46. Docking of compound AD4-1038 to EGFR is depicted, for example, in FIG. 47.

VEGF

Analysis of compounds from the ZINC database against the pharmacophores identified from the 1 CZ8.pdb crystal of protein VEGF (SEQ ID NO: 2) complexed with antibody pertuzumab (see Example 4; Table 18) according to the methods described above, identified compounds including those in Table 24. Glide scores were generated on the hits from the pharmacophore queries described above. Resulting data was arranged according to glide score and 13 AD4 compounds were selected based upon having a g_score of −5.0 (or greater magnitude) plus ZINC02338377 (AD4-2008) (having a g_score=−4.9156) to represent a compound identified using pharmacophore 6n.

TABLE 24

| ZINC# | AD4# | target | G_score | E_model | Pharmacophore model |
|---|---|---|---|---|---|
| ZINC04632336 | AD4-2030 | VEGF | −5.847 | −31.94 | 2n |
| ZINC04618722 | AD4-2025 | VEGF | −5.7582 | −21.61 | 2n |
| ZINC00309762 | AD4-2009 | VEGF | −5.6213 | −39.17 | 4n |
| ZINC00394756 | AD4-2018 | VEGF | −5.502 | −31.2 | 10b |
| ZINC04548161 | AD4-2031 | VEGF | −5.405 | −40.69 | 10b |
| ZINC04813342 | AD4-2026 | VEGF | −5.3473 | −31.7 | 3n |
| ZINC05100656 | AD4-2027 | VEGF | −5.2796 | −31.86 | 3n |
| ZINC04978204 | AD4-2011 | VEGF | −5.2786 | −39.89 | 4n |
| ZINC00185093 | AD4-2028 | VEGF | −5.1386 | −33.25 | 10b |
| ZINC04858568 | AD4-2014 | VEGF | −5.095 | −30.03 | 2n |
| ZINC01795276 | AD4-2024 | VEGF | −5.095 | −36.99 | 2n |
| ZINC02207909 | AD4-2002 | VEGF | −5.0471 | −29.29 | 2n |
| ZINC02338377 | AD4-2008 | VEGF | −4.9156 | −30.83 | 6n |

HER2

Analysis of compounds from the ZINC database against the pharmacophores identified from the 1N8Z.pdb crystal of protein HER2 (SEQ ID NO: 3) complexed with antibody trastuzmab (SEQ ID NO: 7 and SEQ ID NO: 8) (see Example 4; Table 19) according to the methods described above identified, compounds including those in Table 25. Glide scores were generated on the hits from the pharmacophore queries described above. Resulting data was arranged according to glide score and 18 AD4 compounds were selected based upon having a g_score of −6.0 (or greater magnitude) plus ZINC00177228 (AD4-3006) (having a g_score=−5.8263) to represent a compound identified using pharmacophore 3n.

TABLE 25

| ZINC# | AD4# | target | G_score | E_model | Pharmacophore model |
|---|---|---|---|---|---|
| ZINC02431339 | AD4-3047 | HER2 | −7.3043 | −30.04 | 2b |
| ZINC04301095 | AD4-3035 | HER2 | −7.273 | −42.59 | 1b |
| ZINC04844436 | AD4-3048 | HER2 | −7.1972 | −34.22 | 2b |
| ZINC02874992 | AD4-3001 | HER2 | −7.1271 | −35.43 | 2b |
| ZINC02215883 | AD4-3049 | HER2 | −7.0761 | −37.48 | 2b |
| ZINC04085319 | AD4-3050 | HER2 | −7.0274 | −41.7 | 1b |
| ZINC02203252 | AD4-3051 | HER2 | −6.7834 | −35.72 | 1b |
| ZINC02338116 | AD4-3052 | HER2 | −6.7116 | −35.45 | 2b |
| ZINC00069553 | AD4-3005 | HER2 | −6.6966 | −35.83 | 2b |
| ZINC04085335 | AD4-3053 | HER2 | −6.6431 | −35.55 | 1b |
| ZINC05274525 | AD4-3066 | HER2 | −6.6279 | −37.32 | 2b |
| ZINC05052130 | AD4-3036 | HER2 | −6.5488 | −36.83 | 2b |
| ZINC02275796 | AD4-3054 | HER2 | −6.5398 | −31.77 | 2b |
| ZINC02151172 | AD4-3055 | HER2 | −6.2257 | −35.14 | 1b |
| ZINC04934339 | AD4-3010 | HER2 | −6.1942 | −46.86 | 1b |
| ZINC05029084 | AD4-3037 | HER2 | −6.1297 | −31.28 | 2b |
| ZINC00056472 | AD4-3009 | HER2 | −6.1152 | −37.51 | 1b |
| ZINC00177228 | AD4-3006 | HER2 | −5.8263 | −43.84 | 3n |

ErbB2

Analysis of compounds from the ZINC database against the pharmacophores identified from the 1S78.pdb crystal of protein ERBB2 (SEQ ID NO: 4) complexed with antibody pertuzumab (SEQ ID NO: 9 and SEQ ID NO: 10) (see Example 4; Table 19) according to the methods described above, identified compounds including those in Table 26. Glide scores were generated on the hits from the pharmacophore queries described above. Resulting data was arranged according to glide score and 17 AD4 compounds were selected based upon having a g_score of −7.5 (or greater magnitude) plus ZINC01800927 (AD4-3044) (having a g_score=−7.3143) to represent a compound identified using pharmacophore 5n.

TABLE 26

| ZINC# | AD4# | target | G_score | E_model | Pharmacophore model |
|---|---|---|---|---|---|
| ZINC02705114 | AD4-3045 | ErbB2 | −11.291 | −42.61 | 6b |
| ZINC00068737 | AD4-3065 | ErbB2 | −9.9158 | −39.64 | 6b |
| ZINC01237884 | AD4-3040 | ErbB2 | −9.4174 | −37.89 | 6b |
| ZINC02700145 | AD4-3028 | ErbB2 | −8.7023 | −37.78 | 6b |
| ZINC04174810 | AD4-3017 | ErbB2 | −8.4735 | −40.81 | 6b |
| ZINC00206522 | AD4-3025 | ErbB2 | −8.3726 | −44.14 | 6b |
| ZINC02671167 | AD4-3030 | ErbB2 | −8.1816 | −36.3 | 6b |
| ZINC02755700 | AD4-3018 | ErbB2 | −8.1703 | −43.73 | 6b |
| ZINC04065004 | AD4-3041 | ErbB2 | −8.0536 | −50.41 | 6b |
| ZINC00214733 | AD4-3019 | ErbB2 | −8.0259 | −39.94 | 6b |
| ZINC04187766 | AD4-3042 | ErbB2 | −7.7892 | −48.09 | 6b |
| ZINC04825536 | AD4-3031 | ErbB2 | −7.7817 | −44.39 | 6b |
| ZINC04818614 | AD4-3033 | ErbB2 | −7.7392 | −39.97 | 6b |
| ZINC00467700 | AD4-3027 | ErbB2 | −7.6976 | −35.65 | 6b |
| ZINC04551629 | AD4-3063 | ErbB2 | −7.5778 | −37.9 | 6b |
| ZINC01533049 | AD4-3016 | ErbB2 | −7.5731 | −38.17 | 6b |
| ZINC01800927 | AD4-3044 | ErbB2 | −7.3143 | −58.84 | 5n |

### Example 6

### Testing of Identified Compounds from Pharmacophores for EGFR Inhibition

Identified compounds, representing various pharmacaphore models, were tested for ability to inhibit EGFR at 25 μM.

AD4-compounds were identified using pharmacophore models (see Example 4) and then were docked with the binding site of EGFR (SEQ ID NO: 1) that is recognized by defined CDRs of cetuximab. The inhibition of epidermal growth factor binding by AD4-compounds was then determined (NovaScreen BioSciences, Hanover, Md.). Inhibition of EGF binding was determined at 25 μM concentration.

For the inhibitor assays, K_{D} (binding affinity) was 1.04 nM, while Bₘₐₓ (receptor number) was 43.0 fmol/mg tissue (wet weight). Receptor source was rat liver membranes. The radioligand was [¹²⁵I]EGF (150-200 Ci/μg) at a final ligand concentration of 0.36 nM. A non-specific determinant was used as EGF—[100 nM]. The reference compound and positive control was EGF. Reactions were carried out in 10 mM HEPES (pH 7.4) containing 0.1% BSA at 25oC for 60 minutes. The reaction was terminated by rapid vacuum filtration onto glass fiber filters. Radioactivity trapped onto the filters was determined and compared to control values to ascertain any interactions of test compounds with the EGF binding site. The EGF inhibitor assays were modified from, for example, Mukku (1984) J. Biol. Chem. 259, 6543-6546; Duh et al. (1990) World J. Surgery 14, 410-418; Lokeshwar et al. (1989) J. Biol. Chem. 264(32), 19318-19326.

Results of the EGFR inhibition assays for identified compounds representing various pharmacophore models are presented in Table 27.

TABLE 27

| | | EGFR | |
|---|---|---|---|
| STRUCTURE | AD4-NUMBER | INHIBITION | Pharmacophore Model |
| 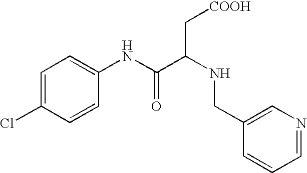 | AD4-1025 | 75.74% | Pharm11_gly54_asp58 |
| 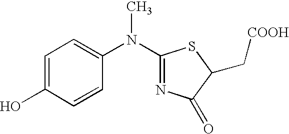 | AD4-1038 | 70.91% | Pharm1_thr100_glu105 |
| 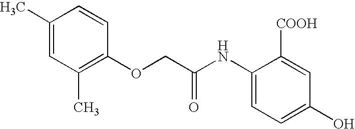 | AD4-1132 | 59.60% | Pharm23_gly54_asp58 |
| 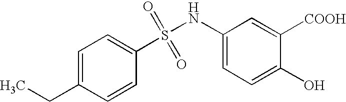 | AD4-1142 | 49.76% | Pharm23_gly54_asp58 |
| 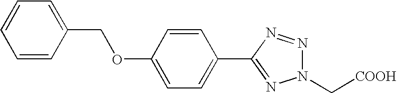 | AD4-1020 | 47.84% | Pharm11_gly54_asp58 |
| 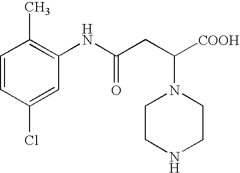 | AD4-1165 | 47.18% | Pharm1_thr100_glu105 |
| 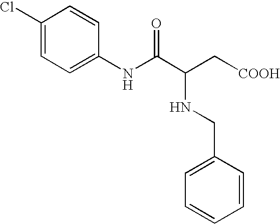 | AD4-1171 | 47.18% | Pharm1_gly54_asp58 |
| 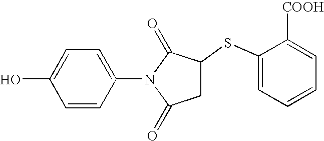 | AD4-1141 | 46.74% | Pharm22_thr100_glu105 |
| 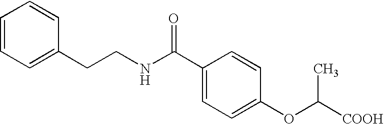 | AD4-1021 | 43.44% | Pharm11_gly54_asp58 |
| 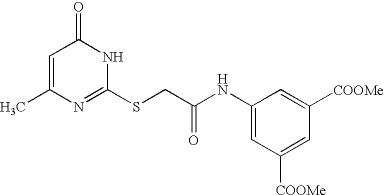 | AD4-1147 | 43.35% | Pharm21_gly54_asp58 |
| 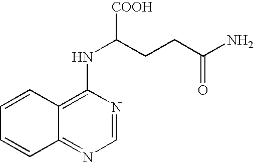 | AD4-1148 | 43.18% | Pharm23_gly54_asp58 |
| 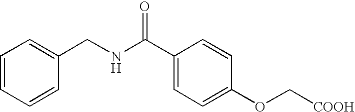 | AD4-1150 | 43.07% | Pharm1_gly54_asp58 |
| 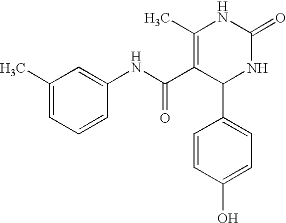 | AD4-1010 | 39.40% | Pharm2_thr100_glu105 |
| 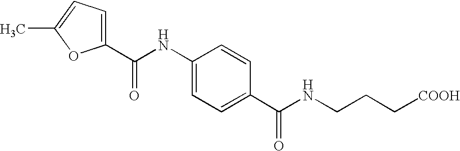 | AD4-1139 | 38.97% | Pharm22_thr100_glu105 |
| 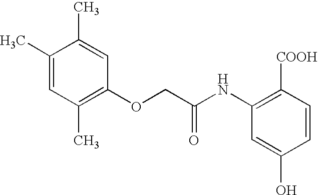 | AD4-1022 | 38.57% | Pharm11_gly54_asp58 |
| 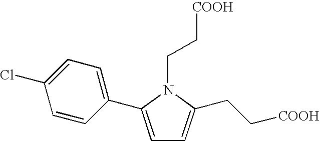 | AD4-1027 | 38.57% | Pharm11_gly54_asp58 |
| 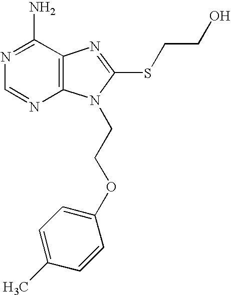 | AD4-1128 | 38.05% | Pharm23_gly54_asp58 |
| 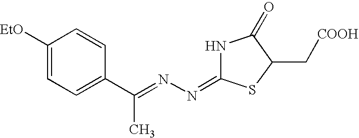 | AD4-1016 | 37.81% | Pharm11_gly54_asp58 |
| 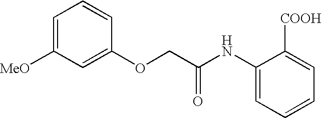 | AD4-1030 | 37.66% | Pharm1_thr100_glu105 |
| 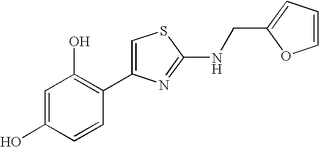 | AD4-1133 | 37.33% | Pharm23_thr100_glu105 |
| 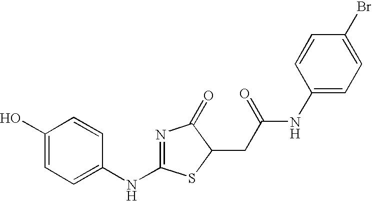 | AD4-1140 | 36.48% | Pharm1_thr100_glu105 |
| 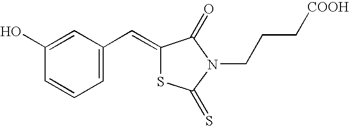 | AD4-1109 | 36.45% | Pharm22_thr100_glu105 |
| 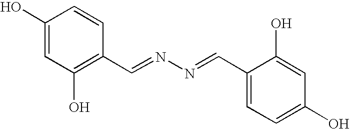 | AD4-1018 | 36.22% | Pharm10_thr100_glu105 |
| 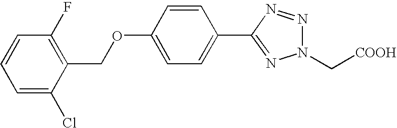 | AD4-1175 | 35.07% | Pharm1_gly54_asp58 |
| 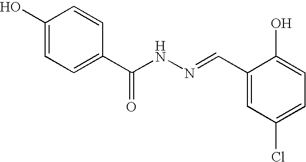 | AD4-1017 | 35.03% | Pharm10_thr100_glu105 |
| 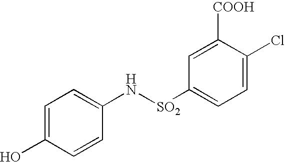 | AD4-1009 | 35.01% | Pharm1_thr100_glu105 |
| 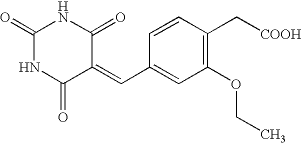 | AD4-1121 | 34.98% | Pharm22_thr100_glu105 |
| 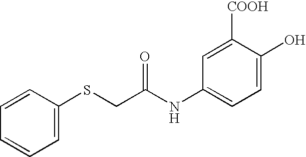 | AD4-1178 | 34.61% | Pharm23_gly54_asp58 |
| 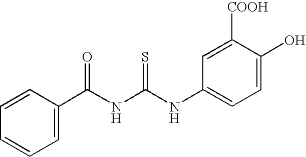 | AD4-1123 | 34.14% | Pharm22_thr100_glu105 |
| 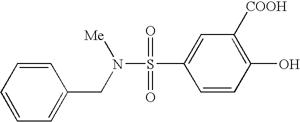 | AD4-1153 | 34.02% | Pharm23_gly54_asp58 |
| 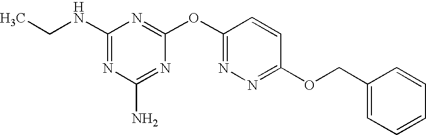 | AD4-1176 | 33.98% | Pharm23_gly54_asp58 |
| 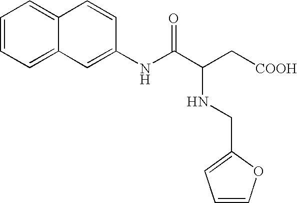 | AD4-1149 | 33.62% | Pharm1_gly54_asp58 |
| 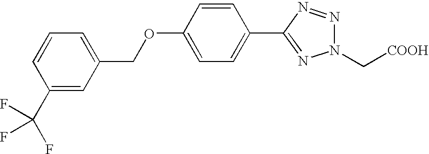 | AD4-1164 | 33.31% | Pharm1_gly54_asp58 |
| 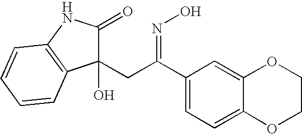 | AD4-1124 | 33.09% | Pharm2l_thr100_glu105 |
| 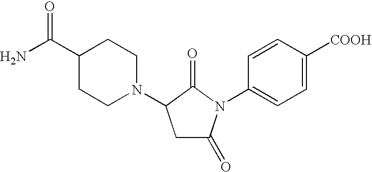 | AD4-1108 | 33.06% | Pharm21_gly54_asp58 |
| 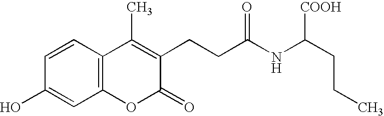 | AD4-1047 | 32.70% | Pharm1_thr100_glu105 |
| 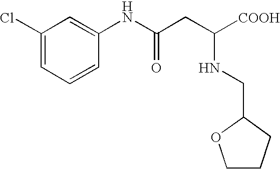 | AD4-1039 | 31.69% | Pharm1_gly54_asp58 |
| 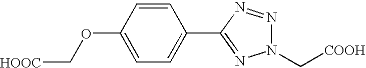 | AD4-1169 | 31.41% | Pharm1_gly54_asp58 |
| 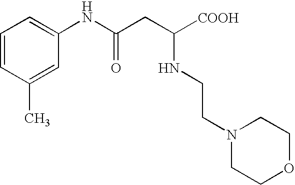 | AD4-1166 | 31.24% | Pharm1_thr100_glu105 |
| 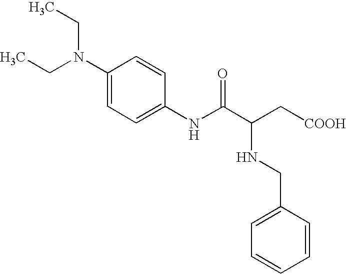 | AD4-1167 | 30.55% | Pharm1_gly54_asp58 |
| 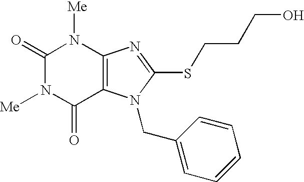 | AD4-1060 | 30.22% | Pharm2_thr100_glu105 |
| 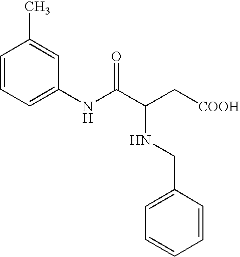 | AD4-1155 | 30.14% | Pharm1_gly54_asp58 |
| 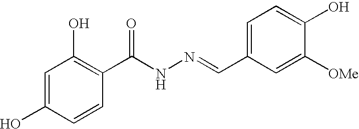 | AD4-1057 | 30.12% | Pharm10_thr100_glu105 |

### Example 7

### AD4-1025 Compound

AD4-1025 (N¹-(4-chlorophenyl)-N²-(3-pyridinylmethyl)-alpha-asparagine; Formula: C₁₆H₁₆ClN₃O₃; Molecular weight: 333.78) is an inhibitor of the binding of epidermal growth factor (EGF) to epidermal growth factor receptor (EGFR (SEQ ID NO: 1)).

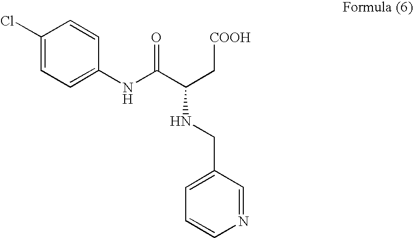

At a concentration of 25 μM of AD4-1025, binding of EGF to EGFR (SEQ ID NO: 1) is inhibited by 75.7% (see e.g., Example 6). The protein crystal structure of cetuximab complexed to EGFR has been reported by Ferguson et al. ((2005) Cancer Cell 7, 301-311) and the crystallographic data deposited in the Protein Data Bank as PDB code 1YY9 (“1YY9.pdb”).

AD4-1025 was identified using information from the 1YY9 protein crystal structure to design a pharmacophore model (see e.g., Example 4). The model, Pharm1_gly54_asp58, was utilized to identify small molecules which bind to EGFR. The site on the EGFR protein is recognized by amino acid residues GLY-54 to ASP-58 of the antibody Cetuximab (SEQ ID NO: 5 and SEQ ID NO:6) (Erbitux). Pharm1_gly54_asp58 is modeled after residues GLY-54 to ASP-58 and designed as a tool to identify small molecules which have features and components of the antibody cetuximab. Specifically this region is defined as the H2CDR of the antibody heavy chain of cetuximab. Features and components of these amino acid residues of cetuximab were used to create a pharmacophore model.

Pharmacophore features (F) and components of Pharm1_gly54_asp58 include: F1:Aro—an aromatic ring center component with a spherical radius of 1.2 Angstroms positioned to interact with ARG353 of EGFR; F2:Aro2—an aromatic ring center component with a spherical radius of 1.5 Angstroms positioned to model the projected directionality to interact with AGR353 of EGFR; F3:Acc&Ani—a hydrogen bond acceptor and anion component with a spherical radii of 1.2 Angstroms positioned to model the carbonyl of GLY-54 of cetuximab; F4:Acc2—a hydrogen bond acceptor component with a spherical radius of 1.5 Angstroms positioned to model the directionality of the lone pair of electrons of the carbonyl group of GLY54 of cetuximab which is seen in the protein crystal structure PDB:1YY9 to engage in a hydrogen bond with ARG353 of EGFR; F5:Acc&Ani—a hydrogen bond acceptor and anion component with a spherical radii of 1.4 Angstroms positioned to model the carboxylate oxygen atoms of ASP-58 of cetuximab; and F6:Acc—a hydrogen bond acceptor component with a spherical radius of 1.2 Angstroms positioned to model the directionality of the lone pair of electrons of the amide carbonyl of THR57 (see e.g., Table 17; FIG. 11).

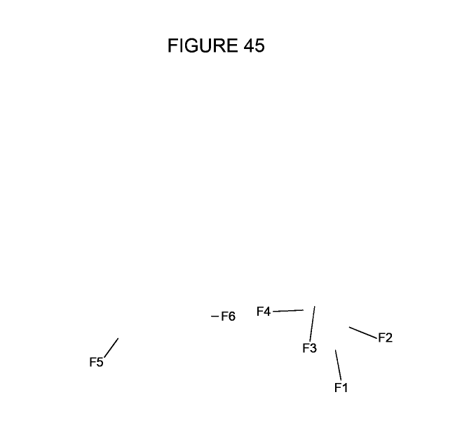

For pharmacophore 10, not all components are essential at one time. The pharmacophore model Pharm1_gly54_asp58 allows for a partial match of 5 of the 6 features and components. Additionally, a feature known as excluded volume constraints is incorporated in Pharm1_gly54_asp58. Excluded volume constraints is used to exclude the space occupied by the target protein, in this case EGFR. To restrict the geometry of the small molecules identified during a pharmacophore query, a group of “dummy” spheres were positioned to occupy the position of atoms of the target protein. These can be seen as the dark grey spheres in FIG. 45. This representation is used to approximate the surface topology of the target protein, EGFR (see e.g., FIG. 45).

Small molecules were identified using a pharmacophore based search of a database of 850,000 commercial compounds (see e.g., Example 4). The compounds identified by Pharm1_gly54_asp58 were then docked, in silico, (see e.g., Example 5) to amino acid residues of the binding site of EGFR (see e.g., FIG. 46) to provide a list of targeted inhibitors.

Using the pharmacophore designated Pharm1_glu54_asp58 to model amino acids GLY54 to ASP58 of cetuximab, compound AD4-1025 was identified. Further testing demonstrated that compound AD4-1025 inhibited EGFR by 76% at 25 μM. An exemplary depiction of AD4-1025 docking with the amino acid residues of the binding site of EGFR is provided in FIG. 46.

Other small molecule EGFR inhibitors identified with Pharm1_glu54_asp58 included: AD4-1020 (48% inhibition at 25 μM); AD4-1021 (43% inhibition at 25 μM); AD4-1027 (39% inhibition at 25 μM); AD4-1022 (39% inhibition at 25 μM); AD4-1030 (38% inhibition at 25 μM); and AD4-1039 (32% inhibition at 25 μM).

### Example 8

### AD4-1038 Compound

AD4-1038 ({2-[(4-Hydroxy-phenyl)-methyl-amino]-4-oxo-4,5-dihydro-thiazol-5-yl}-acetic acid; Formula: C₁₂H₁₂N₂O₄S; Molecular weight: 280.30) is an inhibitor of the binding of epidermal growth factor (EGF) to epidermal growth factor receptor (EGFR (SEQ ID NO: 1)).

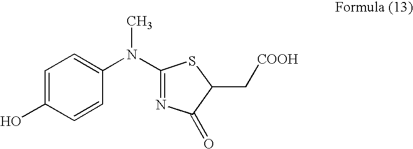

At a concentration of 25 μM of AD4-1038, binding of EGF to EGFR was inhibited by 70.7% (see e.g., Example 6). The model, Pharm1_thr100_glu105, was utilized to identify small molecules which bind to EGFR. The site on the EGFR protein is recognized by amino acid residues THR-100 to GLU-105 of the antibody Cetuximab (SEQ ID NO: 5 and SEQ ID NO:6) (Erbitux). Pharm1_thr100_glu105 was modeled after cetuximab amino acid residues THR-100 to GLU-105 and was designed as a tool to identify small molecules which have features and components of the antibody cetuximab. Specifically, this region is defined as the H3CDR, which is located on the antibody heavy chain of cetuximab. Features and components of these amino acid residues of cetuximab were used to create a pharmacophore model.

Pharmacophore features (F) and components of Pharm1_thr100_glu105 include F1-F8 (see e.g., Table 17; FIG. 17). An exemplary depiction of AD4-1038 docking with the amino acid residues of the binding site of EGFR is provided in FIG. 47. Another small molecule EGFR inhibitor identified with Pharm 1_thr100_glu105 was AD4-1009 (35.01% inhibition at 25 μM).

### Example 9

### AD4-1010 Compound

AD4-1010 (4-(4-hydroxyphenyl)-6-methyl-N-(3-methylphenyl)-2-oxo-1,2,3,4-tetrahydro-5pyrimidinecarboxamide; Formula: C₁₉H₁₉N₃O₃; Molecular weight: 337.37) is an inhibitor of the binding of epidermal growth factor (EGF) to epidermal growth factor receptor (EGFR (SEQ ID NO: 1)).

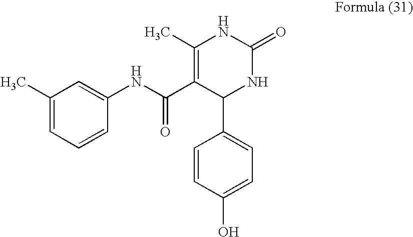

At a concentration of 25 μM of AD4-101, binding of EGF to EGFR is inhibited by 39.40% (see e.g., Example 6). The protein crystal structure of cetuximab complexed to EGFR has been reported by Ferguson et al. ((2005) Cancer Cell 7, 301-311) and the crystallographic data deposited in the Protein Data Bank as PDB code 1YY9 (“1YY9.pdb”).

AD4-1010 was identified using information from the 1YY9 protein crystal structure to design another pharmacophore model. This model was used to identify a different set of EGFR inhibitors. The site on the EGFR protein is recognized by amino acid residues TYR-101 to TYR-104 of the antibody Cetuximab (SEQ ID NO: 5 and SEQ ID NO:6) (Erbitux). Pharm 2_thr100_glu105 was modeled after residues TYR101 to TYR104 and is used to identify small molecules which have features and components of the antibody cetuximab (see e.g., Example 4). Specifically, this region is defined as the H3CDR of the antibody heavy chain. Features and components of these amino acid residues of cetuximab were used to create pharmacophore model Pharm 2_thr100_glu105 (see e.g., Example 4).

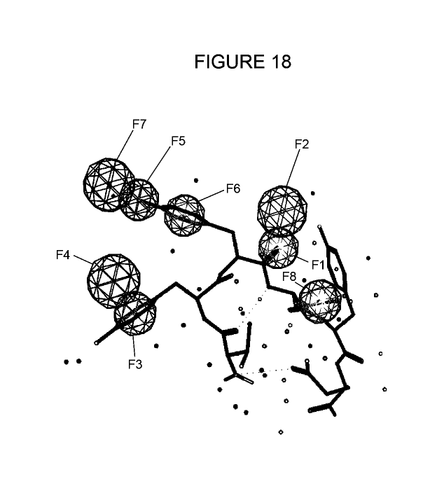

Pharmacophore features (F) and components include: F1:Don&Acc—a hydrogen bond donor and hydrogen bond acceptor component with a spherical radius of 0.8 Angstroms positioned to model the hydroxyl of TYR-102 of cetuximab; F2:Aro—an aromatic ring component with a spherical radius of 1.2 Angstroms positioned to model the phenyl ring of TYR-102 of cetuximab; F3:Acc—a hydrogen bond acceptor component with a spherical radius of 0.8 Angstroms positioned to model the carbonyl oxygen of TYR-102; F4 and F5:Acc&Ani—hydrogen bond acceptors and anion components with a spherical radii of 0.8 angstroms each positioned to model the carboxylate oxygen atoms of ASP-103 of cetuximab; F6:Don&Acc—a hydrogen bond donor and hydrogen bond acceptor component with a spherical radius of 0.8 Angstroms positioned to model the hydroxyl of TYR-104 of cetuximab; and F7:Aro—an aromatic ring component with a spherical radius of 1.2 Angstroms positioned to model the phenyl ring of TYR-104 of cetuximab (see e.g., Table 17; FIG. 18).

For the pharmacophore, not all components are essential at one time. A partial match of 5 of the 7 features and components is allowed. A representation of Pharm 2_thr100_glu105 superimposed with residues TYR-100 to TYR-104 from the protein crystal structure of cetuximab is shown in, for example, FIG. 18.

AD4-1010 was identified by a search of commercial compounds using Pharm 2_thr100_glu105. An exemplary depiction of AD4-1010 docking with the amino acid residues of the binding site of EGFR is provided in FIG. 48.

### Example 10

### AD4-1020

AD4-1020 ({5-[4-(benzyloxy)phenyl]-2H-tetrazol-2-yl}acetic acid; Formula: C₁₆H₁₄N₄O₃; Molecular weight: 310.31) is an inhibitor of epidermal growth factor (EGF) binding to its receptor (EGFR (SEQ ID NO: 1)).

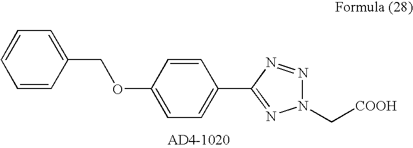

At a concentration of 25 μM of AD4-1020, binding of EGF to EGFR is inhibited by 47.8% (see e.g., Example 6). The protein crystal structure of cetuximab complexed to EGFR has been reported by Ferguson et al. ((2005) Cancer Cell 7, 301-311) and the crystallographic data deposited in the Protein Data Bank as PDB code 1YY9 (“1YY9.pdb”).

AD4-1020 was identified using information from the 1YY9 protein crystal structure to design another pharmacophore model. This model was used to identify a different set of EGFR inhibitors. The site on the EGFR protein is recognized by amino acid residues GLY-54 to ASP-58 of the antibody Cetuximab (SEQ ID NO: 5 and SEQ ID NO:6) (Erbitux). Pharm1_gly54_asp58 is modeled after residues GLY-54 to ASP-58 and is used to identify small molecules which have features and components of the antibody cetuximab (see e.g., Example 4). Specifically, this region is defined as the H2CDR of the antibody heavy chain. Features and components of these amino acid residues of cetuximab were used to create pharmacophore model Pharm1_gly54_asp58 (see e.g., Example 4).

Pharmacophore features (F) and components include: F1 Aro—derived from hydrophobic contact statistics, favorable coulombic interaction with guanidine of Arg353 of the receptor; F2 Aro2—directionality of F1 with respect to guanidine of Arg353; F3 Acc&Ani—derived from Gly54 backbone carbonyl of the antibody cetuximab, acceptor accepts an H-bond from or Anion forms a salt bridge to guanidine of receptor Arg353; F4 Acc2—directionality of F3 with respect to guanidine of Arg353; F5 Acc&Ani—derived from Asp58 side chain carboxylate, acceptor accepts an H-bond from or Anion forms a salt bridge to NH3+ of Lys 443 side chain of the receptor; F6 Acc—derived from hydrophilic contact statistics, accepts an H-bond from side chain OH of Ser448 of the receptor; V1—excluded volumes (not shown for clarity).

For the pharmacophore, not all components are essential at one time. A partial match of 5 of the 6 features and components is allowed. A representation of Pharm1_gly54_asp58 superimposed with residues GLY-54 to ASP-58 from the protein crystal structure of cetuximab is shown in, for example, FIG. 11.

AD4-1020 was identified by a search of commercial compounds using Pharm1_gly54_asp58. An exemplary depiction of AD4-1020 docking with the amino acid residues of the binding site of EGFR is provided in FIG. 53.

### Example 11

### AD4-1132

AD4-1132 ((2-{[(2,4-dimethylphenoxy)acetyl]amino}-5-hydroxybenzoic acid); Formula: C₁₇H₁₇NO₅; Molecular weight: 315.32) is an inhibitor of epidermal growth factor (EGF) binding to its receptor (EGFR (SEQ ID NO: 1)).

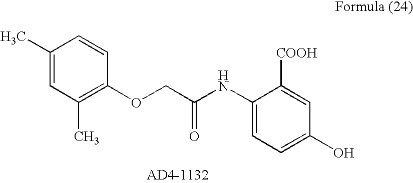

At a concentration of 25 μM of AD4-1132, binding of EGF to EGFR is inhibited by 59.6% (see e.g., Example 6). The protein crystal structure of cetuximab complexed to EGFR has been reported by Ferguson et al. ((2005) Cancer Cell 7, 301-311) and the crystallographic data deposited in the Protein Data Bank as PDB code 1YY9 (“1YY9.pdb”).

AD4-1132 was identified using information from the 1YY9 protein crystal structure to design another pharmacophore model. This model was used to identify a different set of EGFR inhibitors. The site on the EGFR protein is recognized by amino acid residues GLY-54 to ASP-58 of the antibody Cetuximab (SEQ ID NO: 5 and SEQ ID NO:6) (Erbitux). Pharm23_gly54_asp58 is modeled after residues GLY-54 to ASP-58 and is used to identify small molecules which have features and components of the antibody cetuximab (see e.g., Example 4). Specifically, this region is defined as the H2CDR of the antibody heavy chain. Features and components of these amino acid residues of cetuximab were used to create pharmacophore model Pharm23_gly54_asp58 (see e.g., Example 4).

Pharmacophore features (F) and components include: F1 Don—derived from antibody side chain NH2 of Asn56 forms an H-bond with receptor Ser418 side chain OH; F2 Acc&Ani—derived from Gly54 backbone carbonyl of the antibody cetuximab, acceptor accepts an H-bond from or Anion forms a salt bridge to guanidine of receptor Arg353; F3 Acc2—directionality of F3 with respect to guanidine of Arg353; F4 Acc&Ani—derived from antibody Asp58 side chain carboxylate accepting an H-bond from or forming a salt bridge to NH3+ of receptor Lys 443; F5 Don—derived from antibody Gly54 backbone NH forming an H-bond with side chain carbonyl of receptor Gln384; F6 Aro—derived from hydrophobic contact statistics, favorable coulombic interaction with guanidine of Arg353 of the receptor, essential; and F7 Aro2—directionality of F6 with respect to guanidine of Arg353.

For the pharmacophore, not all components are essential at one time. A partial match of 5 of the 7 features and components is allowed. A representation of Pharm23_gly54_asp58 superimposed with residues GLY-54 to ASP-58 from the protein crystal structure of cetuximab is shown in, for example, FIG. 15.

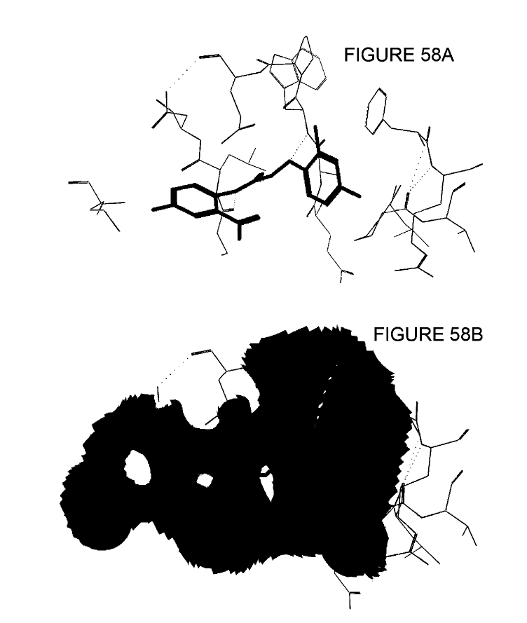

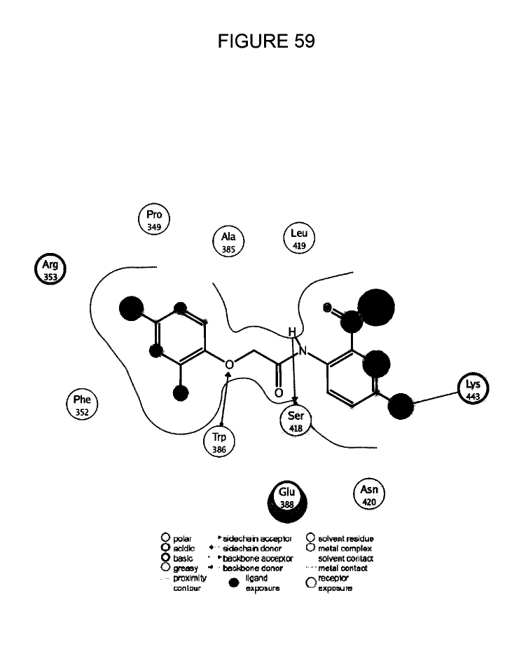

AD4-1132 was identified by a search of commercial compounds using Pharm23_gly54_asp58. An exemplary depiction of AD4-1132 docking with the amino acid residues of the binding site of EGFR is provided in FIGS. 58-59.

### Example 12

### AD4-1142

AD4-1142 ((5-{[(4-ethylphenyl)sulfonyl]amino}-2-hydroxybenzoic acid); Formula: C₁₅H₁₅NO₅S; Molecular weight: 321.35) is an inhibitor of epidermal growth factor (EGF) binding to its receptor (EGFR (SEQ ID NO: 1)). The structure of AD4-1142 is as follows:

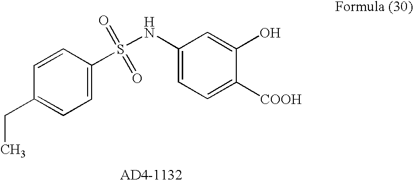

At a concentration of 25 μM of AD4-1142, binding of EGF to EGFR is inhibited by 49.8% (see e.g., Example 6). The protein crystal structure of cetuximab complexed to EGFR has been reported by Ferguson et al. ((2005) Cancer Cell 7, 301-311) and the crystallographic data deposited in the Protein Data Bank as PDB code 1YY9 (“1YY9.pdb”).

AD4-1142 was identified using information from the 1YY9 protein crystal structure to design another pharmacophore model. This model was used to identify a different set of EGFR inhibitors. The site on the EGFR protein is recognized by amino acid residues GLY-54 to ASP-58 of the antibody Cetuximab (SEQ ID NO: 5 and SEQ ID NO:6) (Erbitux). Pharm23_gly54_asp58 is modeled after residues GLY-54 to ASP-58 and is used to identify small molecules which have features and components of the antibody cetuximab (see e.g., Example 4). Specifically, this region is defined as the H2CDR of the antibody heavy chain. Features and components of these amino acid residues of cetuximab were used to create pharmacophore model Pharm23_gly54_asp58 (see e.g., Example 4).

Pharmacophore features (F) and components include: F1 Don—derived from antibody side chain NH2 of Asn56 forms an H-bond with receptor Ser418 side chain OH; F2 Acc&Ani—derived from Gly54 backbone carbonyl of the antibody cetuximab, acceptor accepts an H-bond from or Anion forms a salt bridge to guanidine of receptor Arg353; F3 Acc2—directionality of F3 with respect to guanidine of Arg353; F4 Acc&Ani—derived from antibody Asp58 side chain carboxylate accepting an H-bond from or forming a salt bridge to NH3+ of receptor Lys 443; F5 Don—derived from antibody Gly54 backbone NH forming an H-bond with side chain carbonyl of receptor Gln384; F6 Aro—derived from hydrophobic contact statistics, favorable coulombic interaction with guanidine of Arg353 of the receptor, essential; and F7 Aro2—directionality of F6 with respect to guanidine of Arg353.

For the pharmacophore, not all components are essential at one time. A partial match of 5 of the 7 features and components is allowed. A representation of Pharm23_gly54_asp58 superimposed with residues GLY-54 to ASP-58 from the protein crystal structure of cetuximab is shown in, for example, FIG. 15.

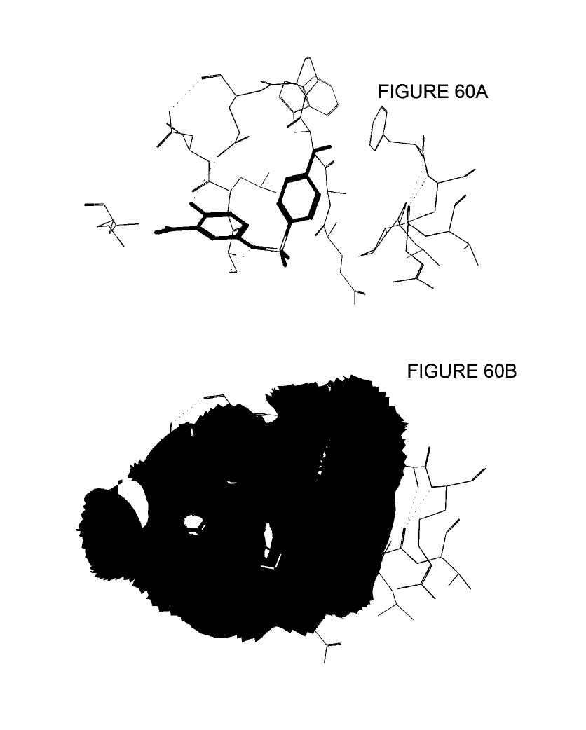

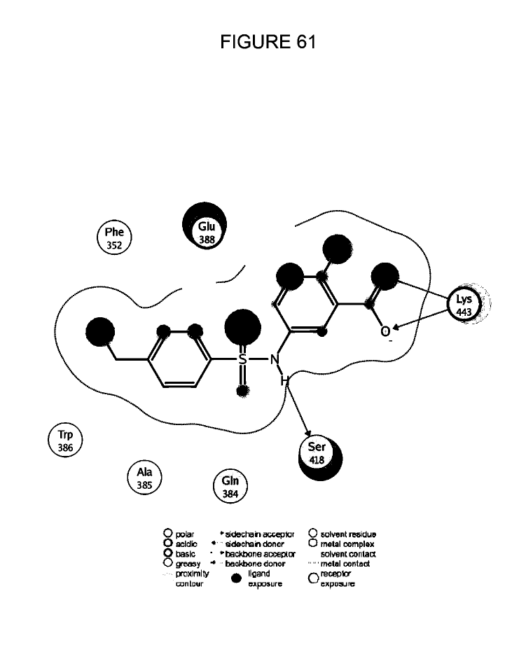

AD4-1142 was identified by a search of commercial compounds using Pharm23_gly54_asp58. An exemplary depiction of AD4-1142 docking with the amino acid residues of the binding site of EGFR is provided in FIGS. 60-61.

What is claimed is:

1. A method for producing a molecular structure having a desired pharmaceutical activity relative to a target biomolecule, comprising the steps of:
providing at least one immune system protein that specifically binds to a target biomolecule;
determining the identity and spatial orientation of at least a portion of atoms of the at least one immune system protein, wherein interaction of the at least a portion of atoms of the at least one immune system protein with a binding site of the target biomolecule result in binding thereto; and
constructing a pharmacophore, wherein the pharmacophore comprises a model of at least one pharmacophoric feature that approximates at least a portion of the identity and spatial orientations of the atoms of the at least one immune system protein that specifically bind to the immune system protein such that the pharmacophore structural features are complementary to the binding site of the target biomolecule.

2. The method of claim 1 further comprising the step of:
identifying a candidate molecule with a pharmacophore hypothesis query of a database of annotated ligand molecules, wherein an identified candidate compound has a structure that substantially aligns with at least one pharmacophoric feature.

3. The method of claim 2 further comprising the step of:
determining a docking affinity of the candidate molecule for the binding site of the target biomolecule;
wherein docking affinity is quantified by energy gained upon interaction of the candidate molecule with the target biomolecule, energy required to attain the docked conformation relative to the lowest energy conformation, or a combination thereof.

4. The method of claim 1 wherein the at least one immune system protein has an ability to alter an activity of the target biomolecule.

5. The method of claim 4 wherein the at least one immune system protein has an ability to inhibit an activity of the target biomolecule.

6. The method of claim 4 wherein the step of providing at least one immune system protein that specifically binds to a target biomolecule and has the ability to alter the activity of the target biomolecule comprises the steps of:
providing an assay in which the target biomolecule displays an activity that mimics an in vivo activity;
exposing a plurality of immune system proteins having a binding affinity for the target biomolecule to the target biomolecule in the assay; and
selecting at least one immune system protein having the ability to alter the activity of the target biomolecule within the assay.

7. The method of claim 1 wherein the at least one immune system protein that specifically binds to the target biomolecule also binds to at least one related biomolecule that differs from the target biomolecule in portions thereof, but wherein similar or identical portions of the structure and activity of the target molecule are retained by the at least one related biomolecule.

8. The method of claim 1, wherein the at least one immune system protein is at least one of the group consisting of a major histocompatibility complex, a T-cell receptor, a β-cell receptor, and an antibody.

9. The method of claim 8 wherein the at least one immune system protein is at least one monoclonal antibody.

10. The method of claim 9 wherein determining the identities and spatial orientations of at least a portion of the atoms of the at least one monoclonal antibody comprises determining the identities and spatial orientations of at least a portion of the atoms of a binding tip of the at least one monoclonal antibody.

11. The method of claim 10 wherein identities and spatial orientations are determined for a substantial portion of the atoms of the binding tip of the at least one monoclonal antibody.

12. The method of claim 1 wherein the pharmacophore features comprise at least one feature selected from the group consisting of hydrophobic, aromatic, a hydrogen bond acceptor, a hydrogen bond donor, a cation, and an anion.

13. The method of claim 1 wherein the target biomolecule is a protein.

14. The method of claim 13 wherein the target biomolecule is an enzyme, a signaling protein, or a receptor protein.

15. The method of claim 1 wherein the target biomolecule is selected from the group consisting of Foot and Mouth Disease, Angiotensin II; ErbB2; Flu Agglutinin; Flu Hemagglutinin; Flu Neuraminidase; Gamma Interferon; HER2; Neisseria Meningitidis; HIV1 Protease; HIV-1 Reverse Transcriptase; Rhinovirus; platelet fibrinogen receptor; Salmonella oligosaccharide; TGF-α; Thrombopoietin; Tissue Factor; Von Willenbrand Factor; VEGF; Coronavirus (SARS); Lyme Disease, HIV GP120; HIV GP41; West Nile Virus; Dihydrofolate reductase; and EGFR.

16. The method of claim 15 wherein the target biomolecule is selected from the group consisting of EGFR, VEGF, HER2, and ErbB2.

17. The method of claim 16 wherein the target biomolecule is EGFR.

18. The method of claim 1, wherein the step of determining the identities and spatial orientations of at least a portion of atoms of the at least one immune system protein comprises analysis of X-ray crystallographic data derived from a crystalline form of the at least one immune system protein.

19. The method of claim 18 wherein the X-ray crystallographic data is derived from a crystalline form of the at least one immune system protein bound to the target biomolecule.

20. The method of claim 1, wherein the step of determining the identity and spatial orientation of at least a portion of atoms of the at least one immune system protein comprises the steps of:
determining the peptide sequence of the at least one immune system protein;
producing a virtual model of the three dimensional structure of the immune system protein; and
analyzing the virtual model of the three dimensional structure of the immune system protein so as to determine the identity and spatial orientation of at least a portion of atoms of the at least one immune system protein that interacts with a binding site of the target biomolecule resulting in binding thereto.

21. A method for producing a molecular entity having a desired pharmaceutical activity relative to a target biomolecule, comprising the steps of:
(i) providing at least one monoclonal antibody;
wherein the at least one monoclonal antibody specifically binds to a target biomolecule and inhibits an activity of the target biomolecule;
wherein the at least one monoclonal antibody comprises a binding tip; and
wherein the binding tip comprises a plurality of atoms that interact with a binding site of the target biomolecule resulting in binding thereto;
(ii) determining identity and spatial orientation of a substantial portion of the binding tip atoms that interact with the binding site of the target biomolecule;
wherein such determination of identity and spatial orientation comprises analysis of X-ray crystallographic data derived from a crystalline form of the at least one monoclonal antibody bound to the target biomolecule;
(iii) constructing a pharmacophore;
wherein the pharmacophore comprises a plurality of pharmacophoric features;
wherein the plurality of pharmacophoric features approximate the identity and spatial orientation of at least about 75% of the at least one monoclonal antibody binding tip atoms that interact with the binding site of the target biomolecule;
wherein the plurality of pharmacophoric features are complementary to the binding site of the target biomolecule; and
wherein the plurality of pharmacophoric features comprise at least one feature selected from the group consisting of hydrophobic, aromatic, a hydrogen bond acceptor, a hydrogen bond donor, a cation, and an anion; and
(iv) identifying a candidate molecule with a pharmacophore hypothesis query of a database of annotated ligand molecules;
wherein an identified candidate compound has a structure that substantially aligns with at least one feature of the pharmacophore;
wherein the candidate molecule inhibits the activity of the target biomolecule; and
wherein the target biomolecule is an enzyme, a signaling protein, or a receptor protein.

22. A pharmaceutical composition for the inhibition of EGFR, the composition comprising at least one EGFR inhibitor selected from the group consisting of Formula (1), Formula (7), Formula (14), Formula (19), and Formula (25), including stereoisomers or polymorphs thereof, and a pharmaceutically acceptable carrier or diluent: 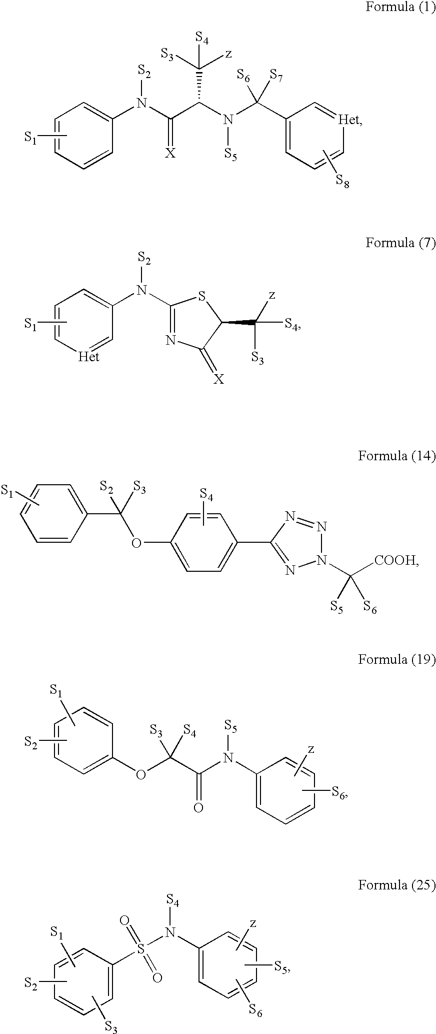
wherein
S1-S8 are independently selected from the group consisting of halogen, hydroxyl, sulfhydryl, carboxylate, alkyl, cycloalkyl, aryl, and alkoxyl (—OR);
X is selected from the group consisting of H₂, O, S, N—R, N—OH, and N—NR₂;
Het is one or more N atoms at any ring position;
Z is selected from the group consisting of —COOH, —PO₃H₂, SO₃H, tetrazole ring, sulfonamide, acyl sulfonamide, —CONH₂, and —CONR₂; and
R is a C1-C6 straight chain or branched alkyl group, optionally substituted with a halogen, hydroxyl, sulfhydryl, carboxylate, aryl, heteroaryl, amino, substituted amino, or cycloamino containing one, two, or three N atoms in a 5 or 6 membered ring.

23. A method for the treatment of a disease or disorder associated with EGFR comprising administering to a mammal in need thereof a composition comprising a therapeutically effective amount of at least one pharmaceutical composition of 22.

24. The method of claim 23, wherein the at least one EGFR inhibitor is selected from the group consisting of Formula (6); Formula (13); Formula (18); Formula (24); and Formula (30), or stereoisomers or polymorphs thereof: [chemical structure]
